(19) United States
(12) Patent Application Publication
(10) Pub. No.: US 2021/0177013 A1
(43) Pub. Date: Jun. 17, 2021

(54) WATER-SOLUBLE FORMULATIONS, METHODS OF MAKING AND USE

(71) Applicant: CANOPY GROWTH CORPORATION, Smith Falls (CA)

(72) Inventors: John Nicholas Jackowetz, Smith Falls (CA); Soheil Hajirahimkhan, Smith Falls (CA); Brandon Pasquariello, Smith Falls (CA); Ben Geiling, Smith Falls (CA); Scott Young, Smith Falls (CA); Matthew Coulter, Smith Falls (CA)

(73) Assignee: CANOPY GROWTH CORPORATION, Smith Falls (CA)

(21) Appl. No.: 17/182,767

(22) Filed: Feb. 23, 2021

Related U.S. Application Data
(63) Continuation-in-part of application No. 16/699,319, filed on Nov. 29, 2019.
(60) Provisional application No. 62/773,644, filed on Nov. 30, 2018.
(60) Provisional application No. 62/773,652, filed on Nov. 30, 2018.
(60) Provisional application No. 62/926,885, filed on Oct. 28, 2019.

(51) Int. Cl. A23L 2/52; A61K 31/05; A61K 31/352; A61K 9/00

(52) U.S. Cl. CPC A23L 2/52; A61K 9/0095; A61K 31/352; A61K 31/05

(57) ABSTRACT

Water-soluble formulations including cannabinoids or a cannabis-derived compound for use in beverages and foods, methods of preparing the formulations, and methods of preparing beverages and foods including the formulations are disclosed herein. In some embodiments, the water-soluble formulations comprise a cannabinoid or a cannabis-derived compound; an emulsifier comprising Quillaja saponins; a carrier oil, and a glycerin-based carrier surfactant.

### CROSS-REFERENCE TO RELATED APPLICATIONS

This application is a continuation-in-part of U.S. patent application Ser. No. 16/699,319 filed on Nov. 29, 2019; which claims the benefit of and priority to U.S. Patent Application No. 62/773,644 filed on Nov. 30, 2018; U.S. Patent Application No. 62/773,652 filed on Nov. 30, 2018; and U.S. Patent Application No. 62/926,885 filed on Oct. 28, 2019, each of which are hereby incorporated by reference in their entirety.

### TECHNICAL FIELD

The present disclosure relates generally to the field of water-soluble formulations, and more particularly to water-soluble formulations comprising cannabinoids or cannabis-derived compounds for use in beverages, topicals and foodstuffs.

### BACKGROUND

In the cannabis industry, an important aspect of preparing a commercial product is the ability to formulate cannabinoids and other cannabis-derived compounds in a desirable form for human consumption.

Smoking is not typically acceptable to non-smokers, as it can be aesthetically unpleasant and can involve health risks such as irritation to at least the mouth, esophagus and lungs. Cigarette smoking has been linked to devastating health risks thought to result from the formation of harmful combustion products. In some jurisdictions, legislation exists which prohibits smoking in various locations and cannabis smoking itself is the target of regulation due to so-called “second hand smoke” risks, as well as what is said to be unpleasant smells for some people. Methods for consuming cannabis, and more particularly cannabinoids, which do not involve smoking or other vaporous means of ingestion may therefore be advantageous as such methods do not involve these and other unwanted effects.

Oral consumption comprises a significant percentage of total cannabis use in federally legal jurisdictions as well as on a state, province, or the like, basis globally. Many orally consumable products, however, contain unhealthy amounts of substances other than cannabis or cannabinoids. Such ingredients include various sugars, caffeine and a variety of non-sugar stimulants, ethanol, and plant-based substances thought to be nutritional supplements, but which have not been the subject of extensive safety testing in complex formulations including cannabis and cannabinoid-containing compositions. Further, many known oral products use expensive gums, which are cost prohibitive and may also have unpredictable supply.

As hydrophobic compounds, cannabinoids and other cannabis-derived compounds present challenges for preparing desirable consumer products, such as beverages and other foodstuffs. Cannabinoids, including many cannabinoid extracts and oils, are insoluble in water thereby making many food products and beverages difficult to produce, including difficulties in obtaining desirable concentrations of cannabinoids in these products.

A need therefore exists for improved water-soluble formulations of cannabinoids that may be used in the preparation of consumer products, and in particular aqueous-based products such as beverages, including sports drink beverages. There further exists a need that these formulations have wide-range applicability in preparing consumer products.

### SUMMARY

The present disclosure provides a convenient water-soluble formulation of cannabinoids or cannabis-derived compounds that may be used in beverages and foodstuffs. More particularly, in select embodiments, the present disclosure provides a formulation of cannabinoids for use in liquid or dispersible powder forms that is soluble in water, and capable of improving the dispersibility and stability of the cannabinoids to provide for acceptable shelf-life of the formulations and products produced therefrom (e.g. beverages).

In some embodiments, the formulation is of natural origin and calorie-free (i.e., less than 5 kcal per serving). In some embodiments, the formulation may advantageously have little or no taste and odor. In particular, in some embodiments, the water-soluble formulations may be used to prepare products that are of clean taste in that the water-soluble formulations do not impart an unpleasant or undesirable taste to the products.

In some aspects, as an alternative or in addition to the cannabinoids, the formulations of the present disclosure may include other cannabis-derived compounds (e.g., cannabis extract, terpenes, etc.), non-cannabis-derived compounds (e.g., non-cannabis terpenes), and/or nutritional supplements (e.g., vitamins) in a single convenient formulation or dosage form.

The present disclosure is directed to water-soluble formulations including cannabinoids or cannabis-derived compounds for use in beverages, foods and other products, and to methods of preparing the formulations. The present disclosure is also directed to foodstuffs and beverages comprising said formulations (e.g. produced using the water-soluble formulations). In particular, the water-soluble formulations comprise a cannabinoid or a cannabis-derived compound, an emulsifier, and a glycerin-based carrier surfactant. In select embodiments, the water-soluble formulations further comprise a carrier oil. The water-soluble formulations may be a liquid or a dispersible powder.

Most suitably and in select embodiments, the formulations are physically and chemically stable; transparent or translucent in colour; calorie-free; and have minimal flavour. Advantageously, in select embodiments, the water-soluble formulations are also transparent or translucent when mixed into an aqueous product, such as a beverage. As used herein, “transparent” is defined by transmittance instruments as known in the art. “Translucent” is defined by either transmittance or reflectance measurement modes (see HunterLab definition, which is available at www.hunterlab.com/transluceent-beverage-color-measurement).

Further, in select embodiments, the formulations include favorable pharmacokinetics, for example, rapid onset, shorter duration, and minimal food effect as described more fully herein.

The present disclosure is also directed to methods of preparing the compositions that are commercially-viable, efficient, and produce shelf-stable formulations and products.

According to one aspect of the present disclosure, there is provided a water-soluble formulation comprising a cannabinoid or a cannabis-derived compound; an emulsifier; and a glycerin-based carrier surfactant. In select embodiments, the water-soluble formulations further comprise a carrier oil. In some embodiments, the carrier oil is comprised of monoglycerides. In some embodiments, the emulsifier comprises a soy lecithin. In some embodiments, the emulsifier comprises a sunflower lecithin. In some embodiments, the glycerin-based carrier surfactant is a vegetable glycerin.

In some embodiments, the water-soluble formulation comprises a cannabinoid distillate or a cannabinoid isolate; monoglycerides; and a soy lecithin; a vegetable glycerin.

In some embodiments, the water-soluble formulation comprises a cannabinoid or a cannabis-derived compound; an emulsifier comprising Quillaja saponins; a carrier oil, and a glycerin-based carrier surfactant.

In some embodiments, the water-soluble formulation comprises up to 10% by weight of the cannabinoid or cannabis-derived compound; up to 10% by weight of the carrier oil, and up to 10% by weight of the emulsifier. In some embodiments, the water-soluble formulation comprises the cannabinoid or cannabis-derived compound; the carrier oil, and the emulsifier at an about equivalent amount by weight. In some embodiments, the water-soluble formulation comprises between about 60% and about 97% by weight of the glycerin-based carrier surfactant.

In some embodiments, the water-soluble formulation comprises a cannabinoid distillate or a cannabinoid isolate; monoglycerides; a soy lecithin; and a sucrose monoester; in a vegetable glycerin. In some embodiments, the water-soluble formulation comprises an about equivalent amount by weight of the soy lecithin and the sucrose monoester.

In some embodiments, the water-soluble formulation is an emulsion. In some embodiments, the water-soluble formulation is clear. In some embodiments, the water-soluble formulation is transparent, translucent, or pearlescent when mixed with an aqueous solution, including when mixed in an aqueous solution.

In some embodiments, the water-soluble formulations comprises a cannabinoid and the cannabinoid is THC (Δ9-THC), Δ8-THC, trans-Δ10-THC, cis-Δ10-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT, cannabicitran, or any combination thereof. In select embodiments, the cannabinoid is CBD, THC or a combination thereof. In select embodiments, the cannabinoid is THC alone or CBD alone.

In some embodiments, the water-soluble formulations comprise a cannabis-derived compound and the cannabis-derived compound is a cannabis-derived cannabinoid, a cannabinoid distillate, a cannabinoid isolate, a terpene, or any combination thereof.

Advantageously, in some embodiments, the water-soluble formulation is shelf-stable at room temperature, including for at least 55 days. In some embodiments, the water-soluble formulation loses less than 20% by weight of the cannabinoid or cannabis derived compound in 3 months.

In some embodiments, when mixed with the aqueous solution, the water-soluble formulation provides a product in which at least 80% by weight of the cannabinoid or cannabis-derived compound remains present after about 2 months at a temperature between about 17° C. and about 40° C., more particularly at least 90%. In some embodiments, when mixed with the aqueous solution, the water-soluble formulation provides a product in which at least 84.89% by weight of the cannabinoid or cannabis derived compound remains present after about 3 months at a temperature of about 40° C.

In some embodiments, the water-soluble formulations may further comprise one or more additives selected from the group consisting of terpenes, terpenoids, flavonoids, viscosity modifiers, natural emulsifiers, oils, thickening agents, minerals, acids, bases, vitamins, flavours, colourants, sweeteners, and combinations thereof. In an embodiment, the water-soluble formulation comprises a terpene having antimicrobial properties.

In another aspect of the present disclosure, there is provided a water-soluble formulation comprising a cannabinoid or a cannabis-derived compound, a carrier oil, a surfactant, and an emulsifier, wherein the water-soluble cannabis formulation is transparent, translucent, or pearlescent when mixed with an aqueous solution.

According to another aspect of the present disclosure, there is provided a powder formulation prepared by drying the water-soluble formulation as described herein. In an embodiment, the powder formulation comprises less than 10 kcal per 250 mg of the powder formulation. More particularly, in an embodiment the present disclosure is directed to a powder formulation prepared by drying a water-soluble formulation as described herein, the water-soluble formulation comprising a cannabinoid or a cannabis-derived compound, a carrier oil, an emulsifier, and a glycerin-based carrier surfactant.

According to another aspect of the present disclosure, there is provided a product comprising the water-soluble formulation as described herein. In an embodiment, the product is a foodstuff. In an embodiment, the product is a beverage and comprises an aqueous solution. In an embodiment, the water-soluble formulation comprises a cannabinoid distillate, monoglycerides, a soy lecithin, and a sucrose monoester, in a vegetable glycerin, and the aqueous solution comprises a stabilizer, for example a chelating agent.

Advantageously, in some embodiments, the product is shelf-stable at room temperature, including for at least 55 days. In some embodiments, the product loses less than 20% by weight of the cannabinoid or cannabis-derived compound in 3 months. In select aspects of these embodiments, the product is a beverage.

In some embodiments, the product is stable in that at least 80% by weight of the cannabinoid or cannabis-derived compound remains present after about 2 months at a temperature between about 17° C. and about 40° C., more particularly at least 90%. In some embodiments, the product is stable in that at least 84.89% by weight of the cannabinoid or cannabis-derived compound remains present after about 3 months at a temperature of about 40° C. In an embodiment, the product has an oxygen content of between about 0 ppm and about 500 ppm.

According to another aspect, the present disclosure is directed to a method for preparing a water-soluble formulation of the present disclosure, the method comprising mixing, in any order, a cannabinoid or a cannabis-derived compound with a glycerin-based carrier surfactant and an emulsifier to prepare the water-soluble formulation.

According to another aspect, the present disclosure is directed to a method of preparing a water-soluble formulation of the present disclosure, the method comprising: mixing a cannabinoid or a cannabis-derived compound and a carrier oil until a homogenous mixture is formed; and mixing a glycerin-based carrier surfactant and emulsifier into the homogenous mixture. In an embodiment, the method further comprises mixing a sucrose monoester into the homogenous mixture.

According to another aspect, the present disclosure is directed to a method for preparing a water-soluble formulation as described herein, the method comprising: mixing a cannabinoid or a cannabis-derived compound with a carrier oil to provide a cannabinoid mixture; mixing a glycerin-based carrier surfactant and an emulsifier comprising Quillaja saponins to provide a glycerin-based mixture; and combining the cannabinoid mixture and the glycerin-based mixture under high shear homogenization to prepare the water-soluble formulation.

According to another aspect, the present disclosure is directed to a method for preparing a powder formulation, the method comprising: mixing a cannabinoid or a cannabis-derived compound and a carrier oil until a homogenous mixture is formed; mixing a glycerin-based carrier surfactant and emulsifier into the homogenous mixture to prepare the water-soluble formulation; and drying the water-soluble formulation.

According to another aspect, the present disclosure is directed to a method for preparing a product comprising a water-soluble formulation of the present disclosure, the method comprising: mixing, in any order, a cannabinoid or a cannabis-derived compound with a glycerin-based carrier surfactant and an emulsifier to prepare the water-soluble formulation; and mixing the water-soluble formulation with an aqueous solution

According to another aspect, the present disclosure is directed to a method of preparing a product comprising a water-soluble cannabis formulation of the present disclosure, the method comprising: mixing a cannabinoid or a cannabis-derived compound and a carrier oil until a homogenous mixture is formed; mixing a glycerin-based carrier surfactant and emulsifier into the homogenous mixture to prepare the water-soluble formulation; and mixing the water-soluble formulation with an aqueous solution. In an embodiment, the product is a beverage. In an embodiment, the method further comprises mixing a sucrose monoester into the homogenous mixture.

In yet another aspect, the present disclosure is directed to a method for preparing a solid product, the method comprising: mixing a cannabinoid or a cannabis-derived compound and a carrier oil until a homogenous mixture is formed; mixing the surfactant and emulsifier into the homogenous mixture to prepare the water-soluble formulation; and absorbing the water-soluble cannabis formulation into a solid material (e.g. tea bag).

Other aspects and features of the water-soluble formulations, methods and products (e.g. dosage forms, beverages and foodstuffs) of the present disclosure will become apparent to those ordinarily skilled in the art upon review of the following description of specific embodiments. Without being bound by any particular theory, the water-soluble formulations of the present disclosure may improve the ability to formulate cannabinoids into aqueous mediums (e.g. beverages and foodstuffs).

### BRIEF DESCRIPTON OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIGS. 1A & 1B depict stability data for water-soluble formulations (FIG. 1A) an end use beverages including the water-soluble formulations (FIG. 1B).

FIGS. 2A-2C are graphs depicting mood experience at 0 hour (FIG. 2A), after 1 hour (FIG. 2B), and after 2 hours (FIG. 2C).

FIGS. 3A-3B are polar area charts to show the complete experience in each category for a Formulation A (FIG. 3A) and a Formulation C (FIG. 3B).

FIG. 4A is a graph depicting intoxication responses for the cannabis formulations.

FIG. 4B is a graph depicting the best fit for intoxication model for the cannabis formulations.

FIG. 4C is a graph depicting blood alcohol concentration over time for comparison to intoxication by cannabis formulations.

FIG. 5 is a graph of intoxication level over time for a participant with cannabis tolerance administered a 6 mg dose of a formulation using Bakerstreet strain without terpenes.

FIG. 6 is a graph of intoxication level over time for a participant with cannabis tolerance administered a 12 mg dose of a formulation using Penelope strain with terpenes.

FIG. 7 is a graph of intoxication level over time for a participant with cannabis tolerance administered a 16 mg dose of a formulation using Penelope strain with terpenes and esters.

FIG. 8 is a graph of intoxication level over time for a participant without cannabis tolerance administered a 12 mg dose of a formulation using Penelope strain with terpenes and esters.

FIG. 9A is a polar area chart showing the sober level of intoxication for a 6 mg dose of a formulation using Bakerstreet strain without terpenes.

FIG. 9B is a polar area chart showing the elevated intoxicated level of intoxication for a 6 mg dose of a formulation using Bakerstreet strain without terpenes.

FIG. 9C is a polar area chart showing the intoxicated level of intoxication for a 6 mg dose of a formulation using Bakerstreet strain without terpenes.

FIG. 10A is a polar area chart showing the sober level of intoxication for a 12 mg dose of a formulation using Penelope with terpenes and esters.

FIG. 10B is a polar area chart showing the elevated intoxicated level of intoxication for a 12 mg dose of a formulation using Penelope with terpenes and esters.

FIG. 100 is a polar area chart showing the intoxicated level of intoxication for a 12 mg dose of a formulation using Penelope with terpenes and esters.

FIG. 11 is a graph showing the loss of cannabinoids for aqueous solutions made using water-soluble formulations of the present disclosure with various different antioxidants, surfactants and emulsifiers.

FIG. 12A is a graph showing the amount of THC remaining in a first beverage prepared using a water-soluble formulation either having or not having sucrose monoester.

FIG. 12B is a graph showing the amount of THC remaining in a first beverage prepared using a water-soluble formulation either having or not having sucrose monoester.

FIG. 13A is a graph showing THC stability data for beverages with varying oxygen concentrations prepared using water-soluble formulations of the present disclosure.

FIG. 13B is a graph showing CBD stability data for beverages with varying oxygen concentrations prepared using water-soluble formulations of the present disclosure.

FIG. 14 is an image showing the clarity/turbidity of aqueous solutions prepared with water-soluble formulations of the present disclosure with and without sucrose monoester.

FIG. 15 is an image showing a gummy product containing a water-soluble formulation of the present disclosure.

FIG. 16 is an image showing a gummy product containing a water-soluble formulation of the present disclosure after 1 week at 25° C.

FIG. 17A is a graph showing the THC and CBD concentration a black tea beverage at 0, 20, 40, 60, 120 and 240 seconds after steeping.

FIG. 17B is a graph showing the THC and CBD concentration a white tea beverage at 0, 20, 40, 60, 120 and 240 seconds after steeping.

FIG. 17C is a graph showing the THC and CBD concentration a herbal tea beverage at 0, 20, 40, 60, 120 and 240 seconds after steeping.

FIG. 17D is a graph showing the THC and CBD concentration a green tea beverage at 0, 20, 40, 60, 120, 240 and 320 seconds after steeping.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the suitable methods and materials are described below.

The present disclosure is generally directed to water-soluble formulations, methods for their preparation, and use thereof. The formulations are suitably in a nontoxic consumable liquid form or a dispersible powder form. The formulations may also be absorbed, sprayed or otherwise applied into or onto a solid material (e.g. a tea bag). Suitably, embodiments of the formulations disclosed herein provide stability, solubility in water, have minimal flavour and odor, are calorie-free, and are natural in origin. In some embodiments, the formulations can contain flavour, odor, and/or calories if desired, particularly when comprised in or used for the preparation of a beverage or foodstuff, including for example a sports drink beverage.

The formulations of the present disclosure include a cannabinoid or a cannabis-derived compound, and in particular, may include a cannabinoid distillate and/or a cannabinoid isolate. Cannabis has been used in beverage preparations for years. Most of the historical cannabis beverages were prepared by boiling or grinding cannabis leaves, combining with water, milk, alcohol, or another biocompatible matrix or beverage liquid and, optionally, mixing with herbal or other plant-based compositions to form the final consumable.

The present disclosure provides improved formulations for cannabinoids and cannabis-derived compounds (e.g. cannabis concentrate, terpenes, etc.). As shown herein, the formulations of the present disclosure comprising cannabinoids, a carrier oil, an emulsifier, and a glycerin-based carrier surfactant are highly soluble in water or a beverage (e.g. Examples 1-7, 9-10 and 12). Thus, the present disclosure provides convenient water-soluble formulations of cannabinoids that may be readily used in the preparation of beverages and foodstuffs (see further Example 8 relating to a gummy product).

The water-soluble formulations of the present disclosure show high emulsion stability evidenced, for example, by the clarity of water-soluble formulations and resultant products such as beverages (e.g. Example 1-2, 5, 7 and 12), as well as the stability of the products and cannabinoids therein (e.g. Examples 2, 6, 10 and 12).

The water-soluble formulations of the present disclosure, and in particular the water-soluble formulations comprising a sucrose monoester, were found suitable for addition to foodstuffs and beverages. In particular, the water-soluble formulations of the present disclosure were capable of preparing beverages that are shelf stable for extended periods of time, e.g. 3 months accelerated stability at 40° C. is roughly representative of 1-year stability at room temperature (e.g. Example 10). The calculated THC and CBD loss at 3-months accelerated testing for beverages 1, 2 and 3 in Example 10 was only 3.64%, 15.11% and 8.71%, respectively. Thus, the water-soluble formulations of the present disclosure are capable of providing excellent cannabinoid stability in beverages.

The water-soluble formulations of the present disclosure, and in particular the water-soluble formulations comprising an emulsifier comprising Quillaja saponins, were found suitable for the preparation of sports drink beverages. Sports drink beverages may present unique formulation challenges for cannabinoids due to the presence of salts (e.g. electrolytes), flavours or other components that may impact stability. Also, certain sports drink beverages may contain higher dissolved oxygen contents than other beverage types and/or may require high pasteurization temperatures in their preparation. The water-soluble formulations of the present disclosure were yet capable of preparing cannabinoid-infused sports drink beverages with desirable physical and chemical stability properties (e.g. Examples 11 and 12).

The water-soluble formulations of the present disclosure, and in particular the water-soluble formulations comprising a sucrose monoester, were also advantageous in preparing transparent, translucent and/or clear beverages over a broad range of different flavour, colour and ionic conditions. For example, as shown in Example 7, across all beverages studied, water-soluble formulations of the present disclosure comprising both soy lecithin and sucrose monoester exhibited a turbidity of less than 5.0 NTU.

The water-soluble formulations of the present disclosure are also advantageous in respect of their preparation. For example, traditional methods of emulsification make use of high shear homogenizers or sonication-based methods that are unable to produce highly uniform particle sizes leading to cloudy, unstable emulsions. Further, while spontaneous formation of cannabinoid containing microemulsions is reported in the literature, these previous methods necessarily used very high concentrations of artificial emulsifiers and were not always fully dilutable, leading to soapy, cloudy, expensive and unpalatable formulations. Additionally, water-soluble cannabinoids may also be produced by molecular encapsulation (i.e., in cyclodextrins and modified starches), but these formulations are usually cloudy and use unnatural ingredients. The presently disclosed water-soluble formulations are generally capable of avoiding such formulation challenges.

Nevertheless, in other aspects, water-soluble formulations of the present disclosure, such as those comprising an emulsifier comprising Quillaja saponins, were also found to be capable of preparing beverages (e.g. sports drink beverages) without the requirement for high pressure microfluidization. Rather, high shear homogenization was found sufficient for preparing cannabinoid-infused sports drink beverages with desirable physical and chemical stability properties (e.g. Examples 11 and 12).

Accordingly, the present disclosure provides convenient water-soluble formulations of cannabinoids that may be readily used in the preparation of beverages and foodstuffs, which are capable of producing beverage products that are transparent, translucent and/or clear and that are shelf stable for extended periods of time.

Individually and separately, these exemplary improvements produce advantageous formulations and dosage forms, and, at times, the combinations of ingredients can provide synergistic beneficial effects on preparation, storage, distribution and/or end use of the formulations. Further improvements are described herein or will become evident from the present disclosure.

### Water-Soluble Formulations

Generally, the water-soluble formulations of the present disclosure include a cannabinoid or cannabis-derived compound (e.g., cannabinoid distillate and/or cannabinoid isolate), a surfactant, and an emulsifier. In a more particular embodiment, the water-soluble formulations further comprise a carrier. Also in a more particular embodiment, the surfactant in the water-soluble formulations is a glycerin-based carrier surfactant.

The water-soluble formulations herein serve as a convenient water-soluble dosage form of cannabinoids for use in beverages, topicals and foods. The formations are suitably in a nontoxic consumable liquid or solid form. Suitably, the formulations provide stability, solubility in water, have minimal flavour and odor, are calorie-free, and are natural in origin.

In an embodiment, the present disclosure relates to a water-soluble formulation comprising a cannabinoid or a cannabis-derived compound; an emulsifier; and a glycerin-based carrier surfactant. In an embodiment, the water-soluble formulation further comprises a carrier oil.

In an embodiment, the present disclosure relates to a water-soluble formulation comprising a cannabinoid or a cannabis-derived compound; an emulsifier comprising Quillaja saponins; a carrier oil, and a glycerin-based carrier surfactant.

In another embodiment, the present disclosure relates to a water-soluble formulation comprising a cannabinoid or a cannabis-derived compound, a carrier oil, a surfactant, and an emulsifier, wherein the water-soluble formulation is transparent, translucent, or pearlescent when mixed with an aqueous solution. In a particular embodiment, the water-soluble formulation is transparent and/or clear.

As used herein, “water-soluble” has its ordinary meaning in referring to the ability of a formulation or component thereof to dissolve when the object is placed in water. For example, when the object is mixed with water at room temperature or slightly above (e.g. about 25° C. to about 50° C.).

As used herein, “transparent” has its ordinary meaning of having the property of allowing light to pass through without appreciable scattering. Transparency may be measured by a transparency meter (also called a clarity meter) and is identified by an object's total transmittance, which is the ratio of transmitted light to the incident light. In an embodiment herein, transparent means a total transmittance of between about 80% and 100%. In an embodiment, transparent means a total transmittance of about 90%, about 91%, about 92%, about 93%, about 94%, about 95%, about 96%, about 97%, about 98%, or about 99%. In an embodiment, transparent means a total transmittance of at least 94%. Visually, the skilled person will appreciate that an object is transparent if it is easy to see through without significant distortion.

Transparency may be equated with clarity (e.g. “clear” or “substantially clear”). As used herein, the term “substantially clear” means that the visible turbidity or cloudiness is very slight (e.g. barely visible to the naked-eye). Turbidity or cloudiness may be measured by a number of means known to one of ordinary skill in the art, including by refractometry, nephelometry, chromatography or spectrometry. In an embodiment, turbidity may be measured by a Nephelometer to determine the Nephelometric Turbidity Units (NTU). In an embodiment, “substantially clear” means that the liquid has an NTU of less than 50. In an embodiment, “substantially clear” means that the liquid has an NTU of ≤25, ≤20, ≤15 or ≤10.

As used herein, the term “clear” means that there is no visible turbidity or cloudiness to the naked-eye. In an embodiment, “clear” means that the liquid has an NTU of ≤5. In an embodiment, “clear” means that the liquid has an NTU of about 5, about 4, about 3, about 2, about 1 or less (e.g. zero).

As opposed to transparent objects and liquids which generally appear clear, as used herein the term “translucent” means that the objects permits the passage of light, but does not appear clear. Translucent objects typically diffuse light such that objects cannot be observed clearly on the opposite side. “Translucent” is defined by either transmittance or reflectance measurement modes (see HunterLab definition, which is available at www.hunterlab.com/transluceent-beverage-color-measurement).

As used herein, “pearlescent” has its ordinary meaning of having a pearly lustre or sheen. Pearlescent may, for example, be used herein to describe a water-soluble formulation that is a powder. In other embodiments, pearlescent may describe the water-soluble formulation as an emulsion, having a shiny lustre when placed in an aqueous formulation.

Cannabis

Cannabis is a genus of flowering plant in the family Cannabaceae. The number of species within the genus is disputed. Three species may be recognized, Cannabis sativa, Cannabis indica and Cannabis ruderalis. C. ruderalis may be included within C. sativa; or all three may be treated as subspecies of a single species, C. sativa. The genus is indigenous to central Asia and the Indian subcontinent.

Cannabis has long been used for hemp fiber, hemp oils, medicinal purposes, and as a recreational drug. Industrial hemp products are made from cannabis plants selected to produce an abundance of fiber. To satisfy the UN Narcotics Convention, some cannabis strains have been bred to produce minimal levels of tetrahydrocannabinol (THC), the principal psychoactive constituent. Many additional plants have been selectively bred to produce a maximum level of THC. Various compounds, including hashish and hash oil, may be extracted from the plant.

Within naturally occurring and man-made hybrids, cannabis contains a vast array of compounds. Three compound classes are of interest within the context of the present disclosure, although other compounds can be present or added to the compositions to augment the experience of a given recreational consumer and medical or medicinal patient or patient population. Those classes include cannabinoids, terpenes and flavonoids.

There are many ways of growing cannabis, some of which are natural, and some are carefully designed by humans, and they will not be recited here. However, one of ordinary skill in the art of cannabis production will typically place a cannabis seed or cutting into a growth media such as soil, manufactured soil designed for cannabis growth or one of many hydroponic growth media. The cannabis seed or cutting is then provided with water, light and, optionally, a nutrient supplement. t times, the atmosphere and temperature are manipulated to aid in the growth process. Typically, the humidity, air to carbon dioxide gas ratio and elevated temperature, either by use of a heat source or waste heat produced by artificial light, are used. On many occasions ventilation is carefully controlled to maintain the conditions described above within an optimal range to both increase the rate of growth and, optionally, maximize the plant's production of the compounds, which comprise the compositions of the disclosure. It is possible to control lighting cycles to optimize various growth parameters of the plant.

Given the number of variables and the complex interaction of the variables, it is possible to develop highly specific formulas for production of cannabis which lead to a variety of desired plant characteristics. The present disclosure is applicable to use with such inventive means for growing cannabis as well as any of the variety of conventional methods.

Cannabis sativa is an annual herbaceous plant in the Cannabis genus. It is a member of a small, but diverse family of flowering plants of the Cannabaceae family. It has been cultivated throughout recorded history, used as a source of industrial fiber, seed oil, food, recreation, religious and spiritual moods and medicine. Each part of the plant is harvested differently, depending on the purpose of its use. The species was first classified by Carl Linnaeus in 1753.

Cannabis indica, formally known as Cannabis sativa forma indica, is an annual plant in the Cannabaceae family. A putative species of the genus Cannabis.

Cannabis ruderalis is a low-THC species of Cannabis, which is native to Central and Eastern Europe and Russia. It is widely debated as to whether C. ruderalis is a sub-species of Cannabis sativa. Many scholars accept Cannabis ruderalis as its own species due to its unique traits and phenotypes that distinguish it from Cannabis indica and Cannabis sativa.

Cannabis-Derived Compounds

As used herein, the term “cannabis-derived compound” refers to a compound found in a cannabis plant, such as for example a compound that has been obtained and/or extracted from cannabis. The method of conversion typically involves harvesting and, optionally, one of the extraction, fractionation, or purification steps described herein. More typically a combination of two or more such steps, more typically yet 2, 3, 4, 5, 6, 7, 8, 9, or 10 individual steps described herein. More typically still a combination of separating the cannabis from the media in which it is grown, drying to reduce the water content, grinding to form a power, extraction and, optionally, a fractionation or purification step is performed.

More typically, the process comprises separation of the cannabis-derived compound from the media in which it is grown followed by 2, 3, 4, or 5 steps as described above are performed, more typically yet, 2, 3, or 4 steps are performed.

Suitably, the cannabis-derived compound is separated from the media in which it is grown and first dried and then ground. Once in the ground state, it is, optionally, sieved and finally the resins of the plant are extracted. These resins comprise the cannabis-derived compounds used in the formulations of the disclosure. Remembering that optional fractionation and purification steps are possible, the formulations of the disclosure may have compounds removed from the resin.

Some steps that can optionally be performed to improve the utility of the compositions include addition, removal or control of the absolute concentrations of compounds comprising the compositions, direct breeding of cannabis strains, genetic manipulation by methods known in the field of molecular biology such as gene insertion or deletion, lyophilization and the development of polyploid variants by use of compounds such as colicine. For example, it is particularly suitable to further refine the resin by molecular distillation to produce a highly concentrated distillate and/or isolate that is substantially free from impurities that might alter taste or smell. As used herein, “substantially free” refers to a compound or composition including less than 1% by weight impurities, suitably less than 0.5% by weight, more suitably less than 0.1% by weight, and even more suitably 0% by weight of an ingredient or component.

Suitable cannabis-derived compounds include, for example and without limitation, cannabis concentrate, cannabis extract, cannabis resin, cannabinoid distillate, cannabinoid isolate, cannabinoids, terpenes, and combinations thereof. Herein, the term “cannabinoid distillate” is used interchangeably with “cannabis distillate”. Also, herein, “cannabinoid isolate” is used interchangeably with “cannabis isolate”. Both a cannabinoid distillate and a cannabinoid isolate comprise one or more cannabinoids. In contrast, in select embodiments, a “cannabis concentrate” or “cannabis extract” may not contain cannabinoids (e.g. a terpene distillate).

In an embodiment, the cannabis-derived compound is a cannabinoid.

In an embodiment, the cannabis-derived compound is a terpene.

In an embodiment, the cannabis-derived compound is a cannabinoid distillate or a cannabinoid isolate. In select embodiments, suitable cannabinoid distillates and isolates for use in the formulations of the present disclosure include distillates and isolates of one or more of the following cannabinoids: Δ9-tetrahydrocannabinolic acid, Δ8-tetrahydrocannabinoilic acid, Δ8-tetrahydrocannabinol, cannabidiolic acid, cannabichromenic acid, Δ9-tetrahydrocannabivarinic acid, Δ9-tetrahydrocannabivarin, cannabigerivarin, cannabidivarin, cannabichromevarin, 11-hydroxy-Δ9-tetrahydrocannabinol, and 11-nor-9-carboxy-Δ9-tetrhydrocannabinol).

Cannabinoids

The compositions of the present disclosure comprise a cannabinoid or a cannabis-derived compound. The cannabis-derived compound may be a cannabinoid, or may be an alternative compound derived from cannabis, such as a terpene.

In an embodiment, the compositions comprise a cannabinoid. The compositions may comprise a single cannabinoid (e.g. THC, CBD or another cannabinoid) or may comprise any combination of two or more cannabinoids (e.g. CBD and THC).

As used herein, the term “cannabinoid” refers to a compound belonging to a class of secondary compounds commonly found in plants of genus cannabis, but also encompasses synthetic and semi-synthetic cannabinoids.

In an embodiment, a cannabinoid is one of a class of diverse chemical compounds that acts on cannabinoid receptors such as CB1 and CB2 in cells that alter neurotransmitter release in the brain. Ligands for these receptor proteins include the endocannabinoids (produced naturally in the body by animals), the phytocannabinoids (found in cannabis and some other plants), and synthetic cannabinoids (manufactured artificially as set forth above). The most notable cannabinoid of the phytocannabinoids is tetrahydrocannabinol (THC), the primary psychoactive compound in cannabis. Cannabidiol (CBD) is another cannabinoid that is a major constituent of the plant. There are at least 113 different cannabinoids isolated from cannabis, exhibiting varied effects.

In one embodiment, the cannabinoid is a compound found in a plant, e.g., a plant of genus cannabis, and is sometimes referred to as a phytocannabinoid. In one embodiment, the cannabinoid is a compound found in a mammal, sometimes called an endocannabinoid. In one embodiment, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

Synthetic cannabinoids and semisynthetic cannabinoids encompass a variety of distinct chemical classes, for example and without limitation: the classical cannabinoids structurally related to THC, the non-classical cannabinoids (cannabimimetics) including the aminoalkylindoles, 1,5-diarylpyrazoles, quinolines, and arylsulfonamides as well as eicosanoids related to endocannabinoids.

In many cases, a cannabinoid can be identified because its chemical name will include the text string “*cannabi*”. However, there are a number of cannabinoids that do not use this nomenclature, such as for example those described herein.

Within the context of this disclosure, where reference is made to a particular cannabinoid, each of the acid and/or decarboxylated forms are contemplated as both single molecules and mixtures. In addition, salts of cannabinoids are also encompassed, such as salts of cannabinoid carboxylic acids.

As well, any and all isomeric, enantiomeric, or optically active derivatives are also encompassed. In particular, where appropriate, reference to a particular cannabinoid incudes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannbinol C4 (THC C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor-9-carboxy-Δ9-tetrahydrocannabinol, Ethoxy-cannabitriolvarin (CBTVE), 10-Ethoxy-9-hydroxy-Δ6α-tetrahydrocannabinol, Cannabitriolvarin (CBTV), 8,9-Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

In an embodiment, the cannabinoid is a cannabinoid dimer. The cannabinoid may be a dimer of the same cannabinoid (e.g. THC-THC) or different cannabinoids. In an embodiment, the cannabinoid may be a dimer of THC, including for example cannabisol.

As used herein, the term “THC” refers to tetrahydrocannabinol. “THC” refers to and is used interchangeably herein with “Δ9-THC”.

In an embodiment, the cannabinoid is THC (Δ9-THC), Δ8-THC, trans-Δ10-THC, cis- Δ10-THC, THCA, THCV, Δ8 THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT, or any combination thereof, each having the following exemplary structural formula:

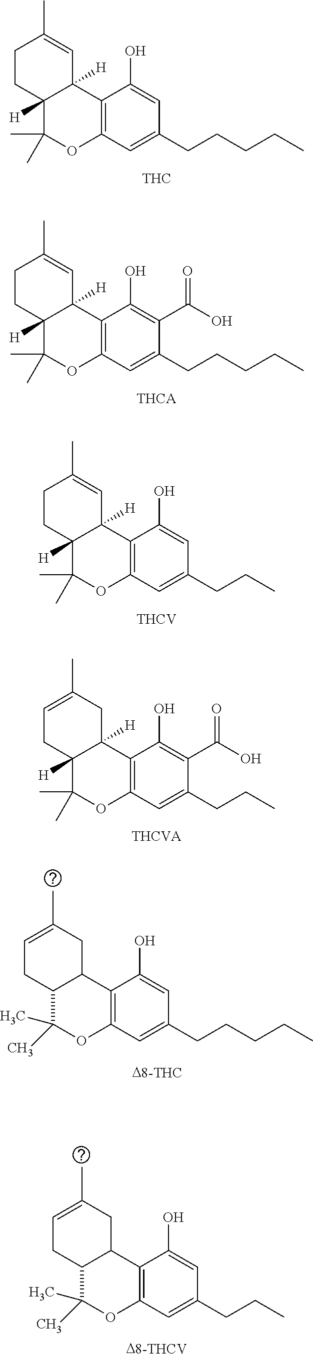

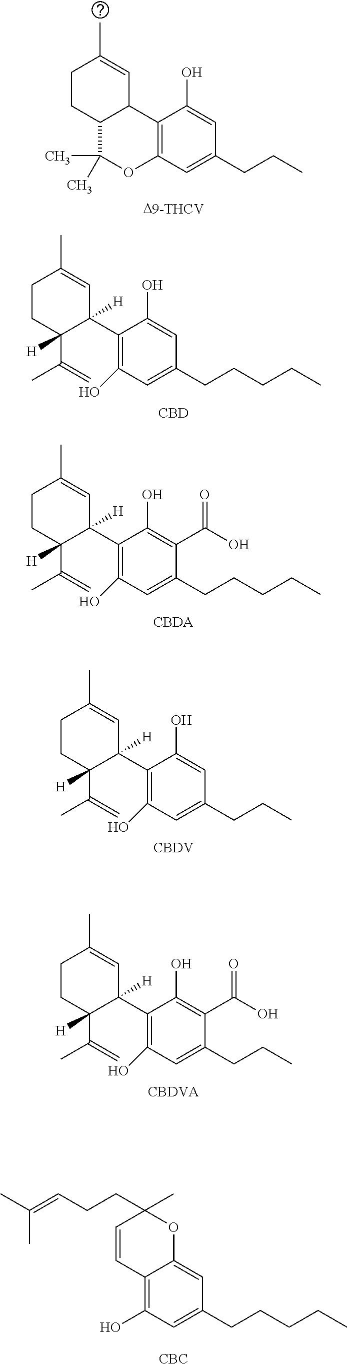

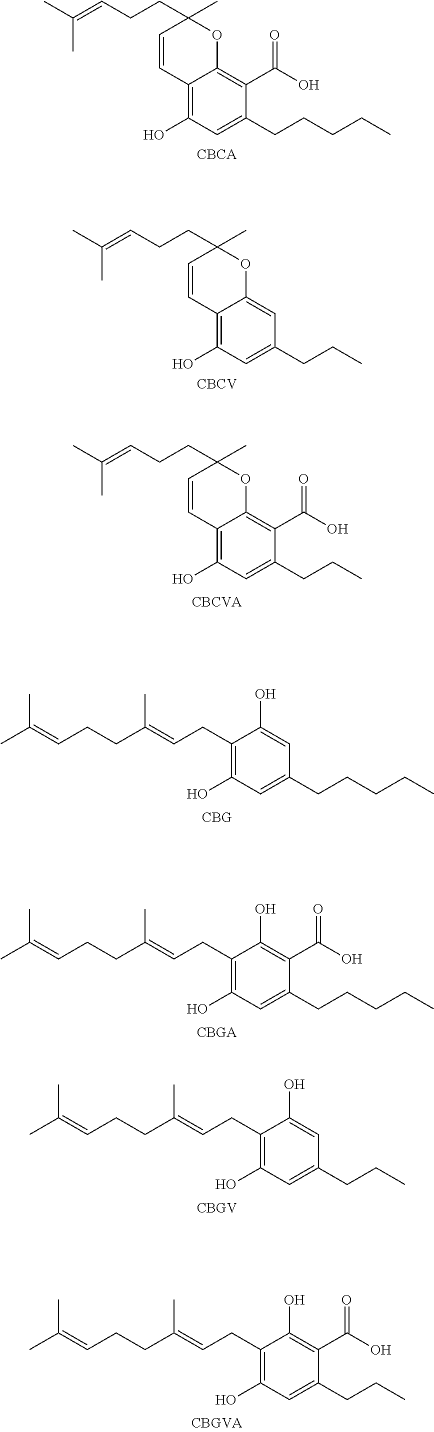

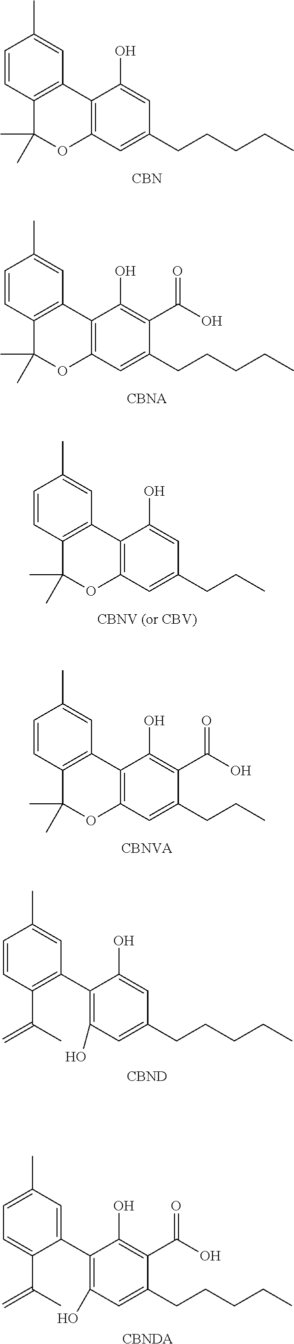

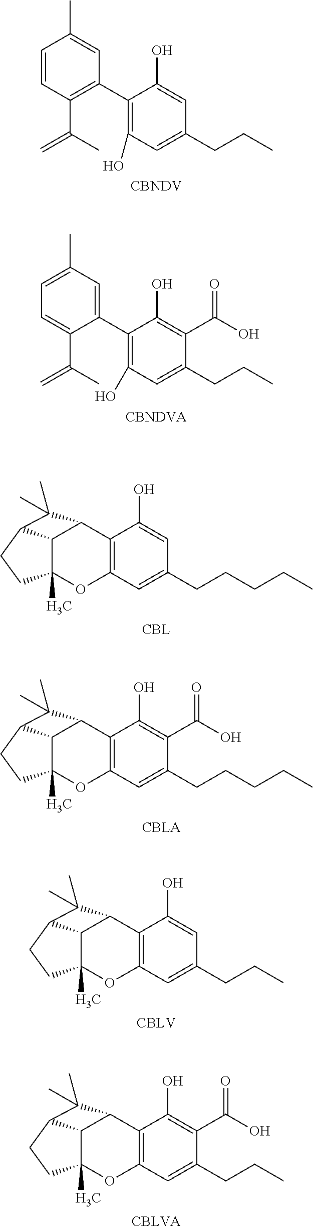

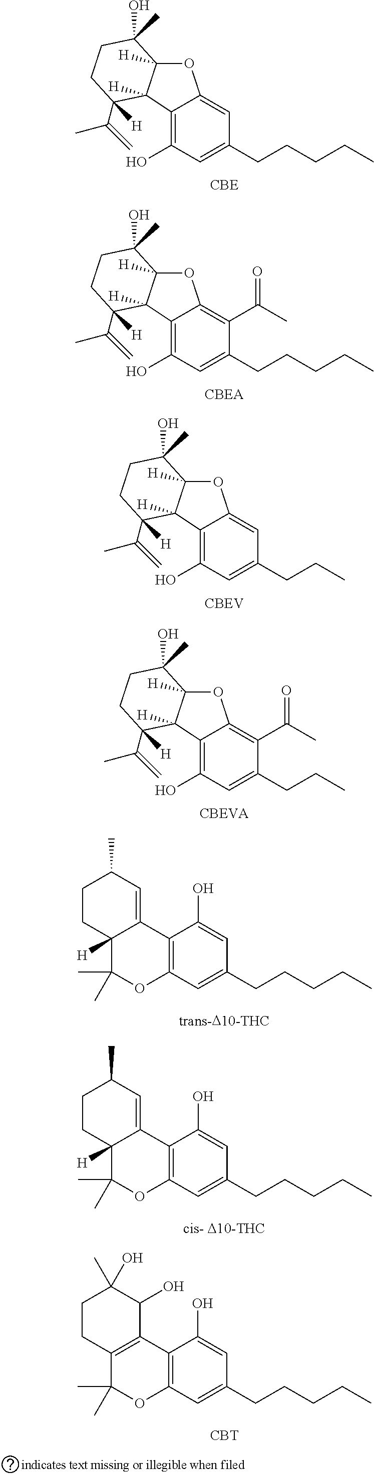

In an embodiment, the cannabinoid is THC, CBD, CBN, CBG, CBGA, or any combination thereof.

Tetrahydrocannabinol (THC) refers to a psychotropic cannabinoid and is the principal psychoactive constituent of cannabis. Its chemical name is (−)-trans-ΔA⁹-tetrahydrocannabinol and the term “THC” is used to refer to isomers as well.

Cannabidiol (CBD) is one of the active cannabinoids identified in cannabis. It is a major phytocannabinoid, by some accounts making up to 40% of the plant's extract. CBD does not appear to have any intoxicating effects such as those caused by THC in marijuana, but may have effects on anxiety, depression and have an anti-psychotic effect, and have effects on other comorbidities. In some instances, the comorbidities are related to disorders described herein such as pain and post-traumatic stress disorders commonly referred to as “PTSD.”

Cannabinol (CBN) is thought to be a non-psychoactive cannabinoid found only in trace amounts in Cannabis and can be produced via oxidative degradation of THCA and THC. Pharmacologically relevant quantities are formed as a metabolite of tetrahydrocannabinol (THC). CBN acts as a partial agonist at the CB1 receptors, but has a higher affinity to CB2 receptors, however; with lower affinities in comparison to THC. Degraded or oxidized cannabis products, such as low-quality baled cannabis and traditionally produced hashish, are high in CBN, but modern production processes have been alleged to minimize the formation of CBN. Cannabinol has been shown to have analgesic properties. Unlike other cannabinoids, CBN does not stem from cannabigerol (CBG).

Cannabigerol (CBG) is thought to be a non-intoxicating cannabinoid found in the Cannabis genus of plants. CBG is the non-acidic form of cannabigerolic acid (CBGA), the parent molecule (“mother cannabinoid”) from which many other cannabinoids are obtained. CBG has been found to act as a high affinity α2-adrenergic receptor agonist, moderate affinity 5-HT1A receptor antagonist, and low affinity CB1 receptor antagonist. It also binds to the CB2 receptor as an antagonist.

Cannabigerolic Acid (CBGA or CBG-A) is the alleged primordial phyto-cannabinoid. It is the alleged compound in cannabis from which all the plant's other naturally occurring cannabinoids are formed; without CBGA, the cannabis plant cannot produce its most useful compounds.

In an embodiment, the cannabinoid is THC (Δ9-THC), Δ8-THC, trans-Δ10-THC, cis-Δ10-THC, CBD, CBC, CBG, CBL, CBN, CBT, cannabicitran, or any combination thereof.

In an embodiment, the cannabinoid is THC or CBD, or a combination thereof.

In an embodiment, the cannabinoid is THC.

In an embodiment, the cannabinoid is CBD.

In an embodiment, the water-soluble formulation may include up to 25% by weight cannabinoid or cannabis-derived compound, and more particularly up to 20%, up to 15%, up to 10% or up to 5% by weight cannabinoid or cannabis-derived compound (e.g., cannabinoid distillate and/or isolate). In select embodiments, the water-soluble formulation may include from about 0.01% by weight to about 25% by weight, from about 0.01% by weight to about 20% by weight, from about 0.01% by weight to about 15% by weight, or from about 0.01% by weight to about 10% by weight, more particularly from about 0.1% by weight to about 8% by weight, even more particularly from about 0.5% by weight to about 5% by weight, and even more particularly still from about 1.0% by weight to about 3% by weight of cannabinoid or cannabis-derived compound. In select embodiments, the water-soluble formulation may include about 0.1%, about 0.25%, about 0.5%, about 0.75%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of cannabinoid or cannabis-derived compound. In a particular embodiment, the water-soluble formulation includes about 1% by weight of cannabinoid or cannabis-derived compound. In one particularly suitable embodiment, the water-soluble formulation includes about 10 mg/mL total cannabinoids.

In select embodiments, the cannabis-derived compound includes one or more cannabinoid distillates and isolates, and in particular, the cannabis-derived compound includes CBD distillates and/or isolates; THC distillates and/or isolates; or a combination of THC and CBD distillates and/or isolates. In some embodiments, the cannabis-derived compounds include THC distillates and/or isolates.

In select embodiments of the water-soluble formulations disclosed herein, the cannabinoids may be introduced in the form of pure cannabinoids or as a cannabis concentrate. As used herein, “pure cannabinoids” is meant to refer to a single cannabinoid or a mixture of different cannabinoids that is free of other compounds. The pure cannabinoids may be contained in solution in a diluent or other medium, or may be a liquid or solid form of the pure cannabinoids absent any diluent. In an embodiment, the pure cannabinoids are synthetic or semi-synthetic cannabinoids. As used herein, “cannabis concentrate” is meant to refer a concentrated composition of cannabinoids, such a cannabinoid extract from a plant. Non-limiting exemplary embodiments of a cannabis concentrate include a cannabinoid distillate, a cannabinoid isolate, a cannabis oil, or any other type of extract containing one or more cannabinoids

As described in greater detail elsewhere herein, in addition to the cannabinoids or cannabis-derived compounds, the formulations of the present disclosure may also include additives, such as for example terpenes, terpenoids, flavonoids, and the like and combinations thereof.

In an embodiment, the additives (e.g. terpenes and/or flavonoids) are independently or in combination derived from natural sources and are selected to be stable in the selected formulations, dosage forms, beverages or foodstuffs herein. More suitably still, in some embodiments, the water-soluble formulation or beverage of the present disclosure with additives is clear, stable at room temperature and capable of being provided in both bulk and unit dose forms. More suitably yet, in some embodiments, the additives may act synergistically in the formulations to provide desirable production, storage, distribution or end use.

Another suitable embodiment of the water-soluble formulations, dosage forms, beverages or foodstuffs of the present disclosure provides fast onset of biological effects of the cannabinoids in human or animal consumers or subjects.

Carrier Oils

In select embodiments, the water-soluble formulations of the present disclosure include at least one carrier oil to reduce the viscosity of the cannabinoids or cannabis-derived compounds and/or provide other suitable properties. Further, at least in the case of solid cannabinoids or cannabis-derived compounds (e.g., crystalline CBD), the carrier oil aids in its dissolution and allows for emulsification of the cannabinoid and cannabis-derived compounds.

Thus, in an embodiment, the present disclosure provides a water-soluble formulation comprising a cannabinoid or a cannabis-derived compound; a carrier oil; an emulsifier; and a glycerin-based carrier surfactant. Water-soluble formulations comprising a carrier oil may represent preferred embodiments, for example having regard to the disclosure herein.

In an embodiment, the carrier oil is an “oily medium”. By “oily medium” it is meant to refer to a medium capable of dissolving lipophilic or hydrophobic compounds, such as cannabinoids. Particularly suitable carrier oils include natural oils as known in the art, for example, edible vegetable oils. In some alternative embodiments, the carrier oils can include synthetic edible oils, for example, hydrogenated vegetable oils, medium chain triglyceride (MCT) oils, and the like and combinations thereof.

A non-limiting list of exemplary carrier oils includes ethanol, isopropanol, dimethyl sulfoxide, acetone, ethyl acetate, pentane, heptane, diethyl ether, medium-chain triglycerides (MCT oil), medium-chain fatty acids (e.g., caproic acid, caprylic acid, capric acid, lauric acid), long-chain triglycerides (LCT oil), long-chain fatty acids (e.g., myristic acid, palmitic acid, stearic acid, arachidic acid, linoleic acid), monoglycerides (e.g. glyceryl monostearate, glyceryl hydroxystearate, glyceryl monoleate, winterized glyceryl monoleate, monolaurin, glyceryl monolinoleate, Maisine® CC, Peceol™), coconut oil, corn oil, canola oil, olive oil, avocado oil, vegetable oil, flaxseed oil, palm oil, palm kernel oil, peanut oil, sunflower oil, rice bran oil, safflower oil, jojoba oil, argan oil, grapeseed oil, castor oil, wheat germ oil, peppermint oil, hemp oil, sesame oil, terpenes, terpenoids, beta-myrcene, linalool, α-pinene, beta-pinene, beta-caryophyllene, caryophyllene oxide, α-humulene, nerolidol, D-limonene, L-limonene, para-cymene, eugenol, farnesol, geraniol, phytol, menthol, terpineol, α-terpineol, benzaldehyde, hexyl acetate, methyl salicylate, eucalyptol, ocimene, terpinolene, α-terpinene, isopulegol, guaiol, α-bisabolol and combinations thereof. Other suitable carrier oils include Labrasol, Labrafac Lipophile WL 1349, Labrafil M1944, Peceol, Plurol Oliqiue CC 497, Transcutol HP, Tween 20, Tween 40, Tween 45, Tween 60, Tween 65, Tween 80, Tween 81, Tween 85, Gelucire 48/16, Vitamin E TPGS, and combinations thereof. In a particularly suitable embodiment, the carrier oil is Maisine® CC.

In an embodiment, a combination of carrier oils may be used in the water-soluble formulations. When more than one carrier is used, they may be used at any amount relative to the other. In an embodiment, the first carrier oil and the second carrier oil may be used at a ratio between 10:1 and 1:10, between 9:1 and 1:9, between 8:1 and 1:8, between 7:1 and 1:7, between 6:1 and 1:6, between 5:1 and 1:5, between 4:1 and 1:4, between 3:1 and 1:3, or between 2:1 and 1:2 by weight to each other. In an embodiment, the two carrier oils may be used at about a ratio of 3:1, 2:1, 1:1, 1:2 or 1:3 by weight to each other. In an embodiment, the two carrier oils may be used at about a 1:1 by weight ratio to each other.

In an embodiment, the water-soluble formulations of the present disclosure and/or products produced therefrom (e.g. beverages or foodstuffs) may include a ratio of carrier oil(s):cannabinoid or cannabis-derived compound of between 10:1 and 1:10, between 9:1 and 1:9, between 8:1 and 1:8, between 7:1 and 1:7, between 6:1 and 1:6, between 5:1 and 1:5, between 4:1 and 1:4, between 3:1 and 1:3, or between 2:1 and 1:2 by weight. In an embodiment, the ratio of carrier oil(s):cannabinoid or cannabis-derived may be about 3:1, 2:1, 1:1, 1:2 or 1:3 by weight. In an embodiment, the ratio of carrier oil(s):cannabinoid or cannabis-derived compound may be about 1:1 by weight.

In an embodiment, the carrier oil may improve the stability of the emulsion, for example by preventing Ostwald ripening of the water-soluble formulation.

In an embodiment, the carrier oil may contribute to providing rapid onset of the cannabinoid or cannabis-derived compound in the water-soluble formulation or a beverage prepared therefrom. The carrier oil may improve the rate of absorption and/or onset of a medicinal, therapeutic and/or recreational effect of the cannabinoids. In an embodiment, the rapid onset occurs within 60 minutes, within 30 minutes, with 15 minutes, or less from administration of the water-soluble formulation to a subject (e.g. in the form of a beverage). The carrier oil may also improve the rate of release of the cannabinoids into a beverage to provide an improved medicinal, therapeutic or recreational effect.

In an embodiment, the carrier oil is comprised of monoglycerides. The monoglycerides may be of a single type (e.g. glyceryl monolinoleate) or may be a mixture of different types. The monoglycerides may include only the monoglyceride ester, or may include one or both of di- and triglycerides. In some embodiments, the monoglyceride fraction is predominant over the di- and triglyceride components. In some embodiments, the di- or triglyceride fractions may be predominant over the monoglycerides, such as for example in Maisine® CC. In an embodiment, the carrier oil is Maisine® CC. In an embodiment, the Maisine® CC contributes to rapid onset of the cannabinoids or cannabis-derived compounds.

In select embodiments, the water-soluble formulations may include up to 20% by weight carrier oil. In an embodiment, the formulations include from about 0.01% by weight to 10% by weight, more particularly from about 0.1% by weight to about 8% by weight, even more particularly from about 0.5% by weight to about 5% by weight, and even more particularly still from about 1.0% by weight to about 3% by weight carrier oil. In select embodiments, the water-soluble formulation may include about 1%, about 2%, about 3%, about 4%, about 5%, about 6%, about 7%, about 8%, about 9%, about 10% by weight carrier oil.

Emulsifiers

The water-soluble formations include one or more emulsifiers to stabilize the mixture of emulsified cannabinoids in the carrier oils described above, to reduce the particle size of the particles in the mixture (e.g. to about 50 nm) and/or to provide other suitable properties.

Any suitable emulsifier may be used. In an embodiment, the emulsifier is an ionic emulsifier. In an embodiment, the emulsifier is a non-ionic emulsifier. In an embodiment, the water-soluble formulations comprise two emulsifiers, where one is an ionic emulsifier and the other is a non-ionic emulsifier.

Without limitation, phospholipids can act as emulsifiers, enabling oils to form a colloid with water. Phospholipids are one of the components of lecithin.

Without limitation, the emulsifier in the water-soluble formulations may be a lecithin. Lecithin is a term commonly used to describe a group of fatty acid substances found in plant and animal tissues. Exemplary sources of lecithin include, without limitation, lecithin which is found in egg-yolks, as well as being extracted from soy beans (i.e., soy lecithin), sunflower (i.e. sunflower lecithin), canola (i.e. canola lecithin), rapeseed (i.e. rapeseed lecithin), cottonseed (i.e. cottonseed lecithin), and animal fats. Lecithin is used as a food additive in many products, and can be purchased as a dietary supplement. Lysolecithins are typically used for water-oil emulsions like margarine, due to their higher HLB ratio.

Any type of lecithin may be used in the practice of the present disclosure. In an embodiment, the lecithin is a soy lecithin, sunflower lecithin, canola lecithin, rapeseed lecithin, cottonseed lecithin, or any combination thereof. In a particular embodiment, the lecithin is a soy lecithin. In an embodiment, the lecithin is a sunflower lecithin.

Other particularly suitable emulsifiers include, for example, members of the ALCOLEC® family of lecithins (e.g. ALCOLEC® F-100, ALCOLEC® EM, ALCOLEC® S, ALCOLEC® BS, ALCOLEC® HL, ALCOLEC® H 20, ALCOLEC® EXTRA-A, ALCOLEC® E 35, ALCOLEC® E 60 or ALCOLEC® HR), including deoiled soy lecithin, sucrose monoesters (e.g. Habo Monoesters P90, SE-50, SE-70, SE-110 or SE-150), GELUCIRE® 48/16, rhamnolipids, LABRASOL®, PLUROL® Oliquie CC, alpha-tocopherol, and combinations thereof.

Other embodiments of emulsifiers may include, for example and without limitation, Gum Arabic, Vitamin E TPGS (e.g. Vitamin E TPGS 200, Vitamin E TPGS 300, Vitamin E TPGS 400, Vitamin E TPGS 1000, Vitamin E TPGS 1500, Vitamin E TPGS 2000 and Vitamin E TPGS 4000), Quillaja extract, PURITY GUM® ULTRA, pectin (e.g. citrus pectin, sugar beet pectin, apple pectin, etc.), chitosan, Q-NATURALE™, and other like compounds.

In an embodiment, the emulsifier in the water-soluble formulations of the present disclosure may be an emulsifier comprises Quillaja saponins. The emulsifier comprising Quillaja saponins may be from any suitable source. As will be appreciated, Quillaja saponins may be derived from a Quillaja tree, such as for example a Quillaja saponaria tree or a Quillaja brasiliensis tree. In a particular embodiment, the emulsifier comprising Quillaja saponins is derived from a Quillaja saponaria tree. In an embodiment, the emulsifier comprising Quillaja saponins is a Q-NATURALE® emulsifier (Ingredion Incorporated, USA), a Saponin from Quallaja Bark (such as for example supplied by Sigma Aldrich, USA), Saponin Quillaja sp. (Sigma Aldrich, USA), a Quillaja extract (such as for example Quillaja extract E 999;), purified Quillaja saponins (e.g. from Sigma Aldrich, USA), a modified Quillaja extract, or combinations thereof.

In an embodiment, the emulsifier comprising Quillaja saponins is a Q-NATURALE® emulsifier. In an embodiment, the Q-NATURALE® emulsifier comprises Q-NATURALE® 200, Q-NATURALE® 200V, or a combination thereof.

In an embodiment, the Q-NATURALE® emulsifier is Q-NATURALE® 200. Q-NATURALE® 200 is described as a natural emulsifier extracted from Quillaja trees and comprising about 65-75% saponin (dry basis) (Ingredion Incorporated, USA). However, modifications and alterations to Q-NATURALE® 200 are also encompassed herein.

In an embodiment, the Q-NATURALE® emulsifier is Q-NATURALE® 200V. Q-NATURALE® 200V is described as a naturally-derived liquid emulsifier derived from the Quillaja Saponaria Molina tree comprising about 13-16% Saponin (wet basis) (Ingredion Incorporated, USA). However, modifications and alterations to Q-NATURALE® 200V are also encompassed herein.

In an embodiment, the water-soluble formulations of the present disclosure comprise a soy lecithin as an emulsifier, alone or in combination with other emulsifiers. In an embodiment, the soy lecithin is ALCOLEC® F-100 or ALCOLEC® EM.

In an embodiment, the water-soluble formulations of the present disclosure comprise a sunflower lecithin as an emulsifier, alone or in combination with other emulsifiers. In an embodiment, the soy lecithin is ALCOLEC® HL or ALCOLEC® H-20.

In an embodiment, the water-soluble formulations of the present disclosure comprise a sucrose monoester as an emulsifier, alone or in combination with other emulsifiers. The sucrose monoester may be sucrose monopalmitate, sucrose monolaurate, sucrose monostearate, or any combination thereof. For any one of these embodiments, the sucrose monoester may comprise a small quantity of diester. In a particular embodiment, the sucrose monoester is sucrose monopalmitate.

In an embodiment, the water-soluble formulations of the present disclosure comprise emulsifier comprising Quillaja saponins as an emulsifier, alone or in combination with other emulsifiers. In an embodiment, the emulsifier comprising Quillaja saponins is Q-NATURALE® 200, Q-NATURALE® 200V, or a combination thereof.

In an embodiment, a combination of emulsifiers may be used in the water-soluble formulations. When more than one emulsifier is used, they may be used at any amount relative to the other. In an embodiment, the first emulsifier and the second emulsifier may be used at a ratio between 20:1 and 1:20, between 15:1 and 1:15, between 10:1 and 1:10, or between 5:1 and 1:5 by weight to each other. In an embodiment, the two emulsifiers may be used at about a 3:1, 2:1, 1:1, 1:2 or 1:3 by weight ratio to each other. In an embodiment, the two emulsifiers may be used at about a 1:1 by weight ratio to each other.

For example, a combination of emulsifiers may be used to strengthen the emulsion as compared to when one emulsifier is used alone. This may be particularly suitable for certain beverages or aqueous solutions, for example where incompatibilities arise between the ingredients of the aqueous solution and the emulsion system. Emulsion instability may arise, for example, due to incompatibilities of a single emulsifier with different pH conditions, different ionic conditions, different oxygen levels, and different packaging materials.

In an embodiment, the water-soluble formulation of the present disclosure includes two or more emulsifiers. In an embodiment, the emulsifiers are selected from lecithins and sucrose monoesters. In an embodiment, the water-soluble formulations comprise two emulsifiers, one selected from a lecithin and one selected from a sucrose monoester. In an embodiment, the two emulsifiers are a soy lecithin and a sucrose monopalmitate. In select embodiments, the lecithin and sucrose monoester are combined in a ratio of between 10:1 and 1:10 (w/w), between 9:1 and 1:9 (w/w), between 8:1 and 1:8 (w/w), between 7:1 and 1:7 (w/w), between 6:1 and 1:6 (w/w), between 5:1 and 1:5 (w/w), between 4:1 and 1:4 (w/w), between 3:1 and 1:3 (w/w), or between 2:1 and 1:2 (w/w). In an embodiment, the lecithin and sucrose monoester are combined in a ratio of about 2:1 (w/w), about 1.5:1 (w/w), about 1:1 (w/w), about (w/w), about 1:1.5 (w/w), or about 1:2 (w/w) of lecithin to SME. In an embodiment, the lecithin and sucrose monoester are combined in a ratio of about 1:1 (w/w).

As shown in Example 5, having two emulsifiers can improve cannabinoid stability and beverage clarity. In addition, particular combinations of emulsifiers can strengthen the emulsion and provide other benefits, such as wider ingredient compatibility. Thus, a single water-soluble formulation may be capable of being used in a broader range of products.

Selection of an emulsifier may affect the shelf-life and physicochemical properties of the water-soluble formulation. Formulations stabilized by surfactants or other types of stabilizing agents such as phospholipids, amphiphilic proteins, or polysaccharides, have been developed to provide controlled release, improved entrapment efficiency, and protection from degradation.

In select embodiments, the water-soluble formulations may include up to about 25% by weight emulsifier, more particularly up to about 15% by weight emulsifier, and more particularly still up to about 12.5% by weight emulsifier. In select embodiments, the water-soluble formulations may include up to 10% by weight emulsifier. In an embodiment, the formulations include from about 0.01% by weight to 10% by weight, more particularly from about 0.1% by weight to about 8% by weight, even more particularly from about 0.5% by weight to about 5% by weight, and even more particularly still from about 1.0% by weight to about 3% by weight emulsifier. In select embodiments, the water-soluble formulation may include about 1%, about 2%, about 3%, about 4%, about 5%, about 6%, about 7%, about 8%, about 9%, about 10%, about 11%, about 12%, about 13%, about 14%, or about 15% by weight emulsifier.

In an embodiment, the water-soluble formulations of the present disclosure and/or the products produced therefrom (e.g. beverages and foodstuffs) may include a ratio of emulsifier(s):cannabinoid or cannabis-derived compound of between 100:1 and 1:10 by weight, between 75:1 and 1:10 by weight, between 50:1 and 1:10 by weight, between 25:1 and 1:10 by weight or between 10:1 and 1:10 by weight. In an embodiment, the ratio of emulsifier(s):cannabinoid or cannabis-derived compound may be about 5:1, 4:1, 3:1, 2:1, 1:1, 1:2, 1:3, 1:4 or 1:5 by weight. In an embodiment, the ratio of emulsifier(s):cannabinoid or cannabis-derived compound may be about 1:1 by weight. In an embodiment, the ratio of emulsifier(s):cannabinoid or cannabis-derived compound may be about 2.5:1 by weight.

Surfactant

The water-soluble formulations of the present disclosure include a surfactant.

Glycerin-Based Carrier Surfactant

In one particularly suitable embodiment, the surfactant is a glycerin-based carrier surfactant. By “carrier surfactant”, it is intended to refer to the feature that the surfactant is the continuous phase (carrier) in which the other components of the water-soluble formulation are dispersed (e.g. the cannabinoids, carrier oil, and emulsifier). It further acts as a surfactant in enabling the formulations of the present disclosure in being water-soluble. By “glycerin-based”, it is meant that the majority component of the surfactant is glycerin. It is envisioned that the glycerin may have other compounds dissolved or suspended therein. Alternatively, the glycerin-based surfactant may be comprised solely of glycerin.

In an embodiment, the glycerin-based carrier surfactant may be present in the water-soluble formulation in an amount between about 10% and about 97% by weight, between about 20% and about 97% by weight, between about 30% and about 97% by weight, between about 40% and about 97% by weight, between about 50% and about 97%, between about 60% and about 97% by weight, between about 70% and about 97%, between about 80% and about 97%, or between about 90% and about 97%.

In a particular embodiment, the glycerin-based carrier surfactant may be present in the water-soluble formulation in an amount between about 60% and about 97% by weight. In select embodiments, the glycerin-based carrier surfactant may be present in the water-soluble formulation in an amount between about 70% and about 97% by weight, more particularly between about 80% and about 97% by weight, and even more particularly between about 90% and about 97% by weight. In an embodiment, the glycerin-based carrier surfactant may be present in the water-soluble formulation in an amount of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, or about 97% by weight.

In an embodiment, the water-soluble formulations of the present disclosure and/or products produced therefrom (e.g. beverages or foodstuffs) may include a ratio of glycerin-based carrier surfactant:cannabinoid or cannabis-derived compound of between 10000:1 and 4:1 by weight, between about 1000:1 and 4:1 by weight, between about 750:1 and about 4:1, between about 500:1 and 4:1 by weight, between about 300:1 and 4:1 by weight, between about 200:1 and 4:1 by weight, between about 100:1 and 4:1 by weight, between about 100:1 and 10:1 by weight, or between about 100:1 and 25:1 by weight. In an embodiment, the ratio of glycerin-based carrier surfactant:cannabinoid or cannabis-derived may be about 250:1, 200:1, 150:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1 or 5:1. In an embodiment, the ratio of glycerin-based carrier surfactant:cannabinoid or cannabis-derived compound may be about 100:1, 99:1, 98:1, 97:1, about 96:1, about 95:1, about 94:1, about 93:1, about 92:1, about 91:1, about 90:1, about 89:1, about 88:1, about 87:1, about 86:1, about 85:1, about 84:1, about 83:1, about 82:1, about 81:1, or about 80:1 by weight.

In an embodiment, the water-soluble formulations of the present disclosure and/or products produced therefrom (e.g. beverages or foodstuffs) may include a ratio of glycerin-based carrier surfactant:emulsifier(s) of between 10000:1 and 4:1 by weight, between about 1000:1 and 4:1 by weight, between about 750:1 and about 4:1, between about 500:1 and 4:1 by weight, between about 300:1 and 4:1 by weight, between about 200:1 and 4:1 by weight, between about 100:1 and 4:1 by weight, between about 100:1 and 10:1 by weight, between about 100:1 and 25:1 by weight. In an embodiment, the ratio of glycerin-based carrier surfactant:emulsifier(s) may be about 250:1, 200:1, 150:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1 or 5:1. In an embodiment, the ratio of glycerin-based carrier surfactant:emulsifier(s) may be about 100:1, 99:1, 98:1, 97:1, about 96:1, about 95:1, about 94:1, about 93:1, about 92:1, about 91:1, about 90:1, about 89:1, about 88:1, about 87:1, about 86:1, about 85:1, about 84:1, about 83:1, about 82:1, about 81:1, or about 80:1 by weight. In an embodiment, the ratio of glycerin-based carrier surfactant:emulsifier(s) may be about 10:1, about 9:1, about 8:1, about 7:1, about 6:1, or about 5:1.

In an embodiment, the water-soluble formulations of the present disclosure and/or products produced therefrom (e.g. beverages or foodstuffs) may include a ratio of glycerin-based carrier surfactant:carrier oil(s) of between 10000:1 and 5:1 by weight, between about 1000:1 and 5:1 by weight, between about 750:1 and about 5:1, between about 500:1 and 5:1 by weight, between about 300:1 and 5:1 by weight, between about 200:1 and 5:1 by weight, between about 100:1 and 5:1 by weight, between about 100:1 and 10:1 by weight, between about 100:1 and 25:1 by weight. In an embodiment, the ratio of glycerin-based carrier surfactant: carrier oil(s) may be about 250:1, 200:1, 150:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1 or 5:1. In an embodiment, the ratio of glycerin-based carrier surfactant: carrier oil(s) may be about 100:1, 99:1, 98:1, 97:1, about 96:1, about 95:1, about 94:1, about 93:1, about 92:1, about 91:1, about 90:1, about 89:1, about 88:1, about 87:1, about 86:1, about 85:1, about 84:1, about 83:1, about 82:1, about 81:1, or about 80:1 by weight.

In an embodiment, the glycerin-based carrier surfactant is glycerin. In an embodiment, the glycerin is a natural or synthetic glycerin. In an embodiment, the glycerin is a vegetable glycerin. As used herein, “vegetable glycerin” refers to the glycerin being made from plant oils. In an embodiment, the vegetable glycerin is made from soybean, coconut or palm oils.

The use of higher amounts of glycerin in the water-soluble formulations of the present disclosure is unusual as typically cannabis-derived formulations are made as oil-in-water emulsions having water as the main component. It was found, however, in the water-soluble formulations of the present disclosure that even when small amounts of water were used as an alternative to glycerin, the resulting formulation was opaque suggesting larger average particle sizes of emulsified cannabinoids within the formulation.

Other Surfactants

In an embodiment, the water-soluble formulations include a surfactant other than a glycerin-based carrier surfactant. The other surfactant may be used as an alternative to the glycerin-based carrier surfactant or in addition to the glycerin-based carrier surfactant.

In an embodiment, the other surfactant is used as an alternative to the glycerin-based carrier surfactant. In such embodiments, most suitably the alternate surfactant is likewise a carrier surfactant. In an embodiment, the other surfactant has chemical, physical and/or functional properties similar to glycerin.

In an embodiment, the other surfactant is used in addition to the glycerin-based carrier surfactant. In an embodiment, the glycerin-based carrier surfactant may be the predominant surfactant by weight (e.g. >50% by weight) or by volume (e.g. >50% by volume). In other embodiments, the glycerin-based carrier surfactant may be the minority surfactant by weight (e.g. <50% by weight) or by volume (e.g. <50% by volume). In an embodiment, there may be 2, 3, 4, 5 or more other surfactants in addition or in alternative to a glycerin-based carrier surfactant.

Other suitable surfactants that can be used as alternatives or in addition to a glycerin-based carrier surfactant include, for example and without limitation, propylene glycol, class 3 solvents (e.g., ethanol, isopropanol), long chain alcohols, terpenes (found in cannabis or not), other poly-alcohols, a polysorbate (e.g. Tween 20, Tween 40, Tween 45, Tween 60, Tween 65, Tween 80, Tween 81 and Tween 85), a polyglyceryi (e.g. polyglyceryl monoesters or polyglyceryl multi-esters), a long chain PEG (e.g. BRIJ® surfactants and Vitamin E TPGS), and the like and combinations thereof.

In an embodiment, where the water-soluble formulations include other surfactants in addition to or in alternative to a glycerin-based carrier surfactant, the total amount of surfactant in the water-soluble formulation may be between about 10% and about 97% by weight, between about 20% and about 97% by weight, between about 30% and about 97% by weight, between about 40% and about 97% by weight, between about 50% and about 97%, between about 60% and about 97% by weight, between about 70% and about 97%, between about 80% and about 97%, or between about 90% and about 97%.

In a particular embodiment, where the water-soluble formulations include other surfactants in addition to or in alternative to a glycerin-based carrier surfactant, the total amount of surfactant in the water-soluble formulation may be between about 60% and about 97% by weight. In select embodiments, the total amount of surfactant in the water-soluble formulation may be between about 70% and about 97% by weight, more particularly between about 80% and about 97% by weight, and even more particularly between about 90% and about 97% by weight. In an embodiment, the total amount of surfactant in the water-soluble formulation may be about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, or about 97% by weight.

In an embodiment, the water-soluble formulations of the present disclosure and/or products produced therefrom (e.g. beverages or foodstuffs) may include a ratio of glycerin-based carrier surfactant:other surfactant(s) of between 1000:1 and 1:1000 by weight, between about 1000:1 and 1:100 by weight, between about 1000:1 and 1:10 by weight, between about 1000:1 and 1:1 by weight, between 100:1 and 1:1000 by weight, between 10:1 and 1:1000 by weight, between 1:1 and 1:1000 by weight, between 100:1 and 1:100 by weight, between about 100:1 and 1:1 by weight, between 1:1 and 1:100 by weight, between 10:1 and 1:10 by weight, between about 10:1 and 1:1, between 1:1 and 1:10 by weight, between 5:1 and 1:5 by weight. In an embodiment, the ratio of glycerin-based carrier surfactant:other surfactant(s) may be about 1000:1, 750:1, 500:1, 250:1, 200:1, 150:1, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, or 1:1.

In an embodiment, the present disclosure relates to a water-soluble formulation comprising a cannabinoid or cannabis-derived compound, a carrier oil, an emulsifier, and a glycerin-based carrier surfactant.

In select embodiments, the water-soluble formulations of the present disclosure comprise the cannabinoid or cannabis-derived compound; the carrier oil; and the emulsifier at an about equivalent amount by weight. In an embodiment, the water-soluble formulations comprise up to 10% by weight of the cannabinoid or cannabis-derived compound; up to 10% by weight of the carrier oil, and up to 10% by weight of the emulsifier. In an embodiment, the water-soluble formulations comprise about 1%, about 2%, about 3%, about 4%, about 5%, about 6%, about 7%, about 8%, about 9%, or about 10% by weight of the cannabinoid or cannabis-derived compound, the carrier oil, and the emulsifier.

In an embodiment, the present disclosure relates to a water-soluble formulation comprising a cannabinoid or cannabis-derived compound, a carrier oil, a first emulsifier, a second emulsifier, and a glycerin-based carrier surfactant.

In an embodiment, the present disclosure relates to a water-soluble formulation comprising a cannabinoid or a cannabis-derived compound, monoglycerides, soy lecithin, sucrose monoester, all in a vegetable glycerin.

In an embodiment, the present disclosure relates to a water-soluble formulation comprising a cannabinoid distillate or a cannabinoid isolate; monoglycerides; a soy lecithin; and a sucrose monoester; in a vegetable glycerin.

In an embodiment, the water-soluble formulations of the present disclosure comprise an about equivalent amount by weight of the soy lecithin and the sucrose monoester.

In an embodiment, the water-soluble formulations comprise up to 10% by weight of each of the cannabinoid or cannabis-derived compound; the carrier oil, the soy lecithin; and the sucrose monoester. In an embodiment, the water-soluble formulations comprise an about equivalent amount by weight of each of the cannabinoid or cannabis-derived compound; the monoglycerides; the soy lecithin; and the sucrose monoester. In an embodiment, the equivalent amount is about 1%, about 2%, about 3%, about 4%, about 5%, about 6%, about 7%, about 8%, about 9%, or about 10% by weight of water-soluble formulation.

In an embodiment, the present disclosure relates to a water-soluble formulation comprising a cannabinoid or a cannabis-derived compound; an emulsifier comprising Quillaja saponins; a carrier oil, and a glycerin-based carrier surfactant. In an embodiment, the water-soluble formulations comprise up to 10% by weight of the cannabinoid or cannabis-derived compound; up to 10% by weight of the carrier oil, up to 15% by weight of the emulsifier comprising Quillaja saponins; and at least 60% by weight of the glycerin-based carrier surfactant. In an embodiment, the ratio of glycerin-based carrier surfactant:emulsifier comprising Quillaja saponins in the water-soluble formulation or product produced therefrom is about 25:1, about 20:1, about 15:1, about 10:1, about 9:1, about 8:1, about 7:1, about 6:1, or about 5:1. In an embodiment, the ratio of glycerin-based carrier surfactant:carrier oil is about 75:1, about 70:1, about 65:1, about 60:1, about 55:1, about 50:1, about 45:1, about 40:1, about 35:1, about 30:1, or about 25:1.

In an embodiment, the water-soluble formulations of the present disclosure are liquid, such as an emulsion. The term “emulsion” is well known in the art and refers to a mixture of two or more liquids that are normally immiscible (unmixable or unblendable), where a first liquid is dispersed in small globules (internal or discontinuous phase) throughout a second liquid (external or continuous phase).

In an embodiment, the water-soluble formulations of the present disclosure are a powder. The powder may, for example, be prepared by drying the liquid water-soluble formulations of the present disclosure. In an embodiment, the powder is of low calorie (e.g. less than 10 kcal per 250 mg of the powder formulation).

Stabilizers

In select embodiments, the water-soluble formulations of the present disclosure may be used in combination with a stabilizer. The stabilizer may be added to the water-soluble formulation or to a product (e.g. aqueous solution, beverage, topical or food) that is produced using the water-soluble formulations.

Thus, in an embodiment, the water-soluble formulations of the present disclosure comprise a stabilizer. In an embodiment, a product produced from the water-soluble formulations comprises a stabilizer. The stabilizer may be added to the product before, during or after admixture with the water-soluble formulation.

As used herein, a stabilizer is any substance used to prevent an unwanted change in state in the water-soluble formulation or product produced therefrom (e.g. prevent degradation). The stabilizer may be used to improve or maintain the stability of the water-soluble formulation itself (e.g. the emulsion) or to improve or maintain the stability of individual components of the water-soluble formulation or product (e.g. the cannabinoids). For example, cannabinoids or cannabis-derived compounds within the water-soluble formulation or product produced therefrom may be susceptible to degradation, such as oxidative degradation. Thus, in an embodiment, the stabilizer protects the cannabinoids or cannabis-derived compounds from degradation.

Non-limiting examples of stabilizers include hydrocolloids (such as alginate, agar, carrageenan, cellulose and cellulose derivatives, gelatin, guar gum, gum Arabic, locust bean gum, pectin, starch and xanthan gum), antioxidants (water-soluble and/or oil-soluble), and chelating agents.

Water-soluble antioxidants may enhance the stability of the water-soluble formulation and/or products containing the water-soluble formulation by reacting with reactive species in the polar (e.g. aqueous) phase. Oil-soluble antioxidants may enhance the chemical stability of the water-soluble formulation and/or products containing the water-soluble formulation by reacting with reactive species in the oil phase. Non-limiting examples of reactive species include peroxides, free radicals and oxygen.

Non-limiting examples of antioxidants include ascorbic acid, ascorbic acid-6 palmitate (ascorbyl palmitate), ascorbyl stearate, alpha-tocopherol, beta-carotene, butylated hydroxyaniline (BHA), butylated hydroxytolulene (BHT), delta-tocopherol, dodecyl gallate, erythorbic acid, gamma-tocopherol, glutathione, lipoic acid, octyl gallate, propyl gallate, mixed tocopherols (e.g. Fortium®), vitamin E (e.g. Tocobiol® Plus CP60), Tocobiol® Plus L-70, Tocobiol® Plus GP, Tocobiol® Plus PV, Nutrabiol T, sodium ascorbate,sodium erythorbate, and Extract of Rosemary (OxiKan® CL).

In an embodiment, the water-soluble formulations of the present disclosure are used in combination with an antioxidant stabilizer. In an embodiment, the antioxidant stabilizer is ascorbic acid-6 palmitate (E-304) or a tocopherol.

Chelating agents may enhance the chemical stability of the water-soluble formulation and/or products containing the water-soluble formulation by binding dissolved metal ions. Dissolved metal ions, for example copper ions or iron ions, may catalyze oxidation-reduction reactions (redox) between dissolved oxygen and the components of the water-soluble formulation or product. In particular, cannabinoids may be susceptible to oxidation catalyzed by dissolved metal ions. Non-exclusive examples of chelating agents include: aminopolycarboxylic acids including ethylenediaminetetraacetic acid (EDTA) and its various salts, calixarenes, porphyrins, bipyridines, citric acid, iminodisuccinic acid, and polyaspartic acid.

In an embodiment, the water-soluble formulations of the present disclosure are used in combination with a chelating agent as a stabilizer. In an embodiment, the chelating agent is ethylenediaminetetraacetic acid (EDTA). In an embodiment, the EDTA is disodium EDTA, calcium disodium EDTA, or tetrasodium EDTA. In a particular embodiment, the EDTA is calcium disodium EDTA.

The stabilizer may be added to the water-soluble formulation or to the product produced therewith. In an embodiment, the stabilizer is added to the product separately from the water-soluble formulation. In an embodiment, the stabilizer is added to the product within 30 minutes before or after the water-soluble formulation is added.

In an embodiment, the stabilizer is added to the water-soluble formulation. In an embodiment, the stabilizer is added to the water-soluble formulation within 30 minutes before the water-soluble formulation is added to the product. In an embodiment, the stabilizer is added to the water-soluble formulation within 1 minute before the water-soluble formulation is added to the product.

The stabilizer may be added to the water-soluble formulation or to the product produced therewith at any suitable concentration. In an embodiment, the stabilizer is added in a minor amount. As used herein, by “in a minor amount”, it is meant that the stabilizer is added to the water-soluble formulation or to the resultant product at a concentration of between 1 ppm and 100 ppm, between 10 ppm and 50 ppm, or between 20 ppm and 30 ppm.

The use of a stabilizing agent is sometimes to the detriment of other important characteristics of a consumer product, e.g. a beverage. For example, additional components such as stabilizers may promote turbidity, cloudiness or an undesired taste profile in the final product. Also, stabilization of one component (e.g. the emulsion) may have a negative effect on the stability of another component (e.g. the active ingredient). This may be particularly so for emulsification products where the development of a water-soluble formulation that is clear in appearance, easy to drink (“clean” taste profile), shelf stable, and quick acting are all relevant considerations.

In an embodiment, the water-soluble formulations of the present disclosure are used in combination with a stabilizer. In an embodiment, the stabilizer is one that complements one or more components of the water-soluble formulation to provide a product that is clear in appearance, chemically stable, shelf stable, and/or suitable for use in a broad range of product having different characteristics (e.g. pH, high or low ionic conditions, wide array of ingredients, etc.). By “chemically stable”, it is meant that the stability of the active ingredient is improved.

In an embodiment, a chelating agent is used in combination with a water-soluble formulation of the present disclosure. In an embodiment, the water-soluble formulation is one that comprises one or both of an emulsifier selected from lecithin and sucrose monoester. In an embodiment, a chelating agent is used in combination with a water-soluble formulation of the present disclosure that comprises both lecithin and sucrose monoester as emulsifiers. In an embodiment, the chelating agent is EDTA.

As shown herein, sucrose monoester and a chelating agent appear to complement each other in that the sucrose monoester strengthens the emulsion (e.g. stabilizes the emulsion) as evidenced by improved clarity, while the chelating agent stabilizes the cannabinoids. This is an advantageous result since it was found that a chelating agent renders the aqueous product more turbid. Combined with a sucrose monoester, the clarity of the product improves (see e.g. Example 5).

The combination of sucrose monoester and lecithin provide better clarity and stability then either alone, and the inclusion of a chelating agent in the aqueous solution appears to provide even greater protection to the cannabinoid, without sacrificing other key characteristics of the product. As seen in Example 4, the chelating agent was found to provide significant protection of THC and CBD from degradation.

In an embodiment, the present disclosure relates to a water-soluble formulation comprising a cannabinoid or cannabis-derived compound, a carrier oil, an emulsifier, and a glycerin-based carrier surfactant, which is used in combination with a stabilizer to prepare a beverage, topical or food.

In an embodiment, the present disclosure relates to a water-soluble formulation comprising a cannabinoid or cannabis-derived compound, a carrier oil, a first emulsifier, a second emulsifier, and a glycerin-based carrier surfactant, which is used in combination with a stabilizer to prepare a beverage, topical or food.

In an embodiment, the present disclosure relates to a water-soluble formulation comprising a cannabinoid or a cannabis-derived compound, monoglycerides, soy lecithin, sucrose monoester, all in a vegetable glycerin, which is used in combination with a chelating agent to prepare a beverage, topical or food.

The water-soluble formulation and/or the products containing the water-soluble formulations may be treated to reduce the oxygen content as this may further enhance their chemical stability. For example, it was found that the rate of oxidative degradation of cannabinoids depends on the oxygen concentration in the product containing the water-soluble formulation. Reducing the oxygen concentration may thereby reduce the rate of oxidative degradation and enhance the chemical stability of the water-soluble formulation and/or the products containing the water-soluble formulation. Non-limiting examples of treatments to reduce oxygen concentration include: sparging (with nitrogen and/or other inert gases), freeze-pump-thaw cycling, and treatment with reducing agents (e.g. potassium metabisulphite).

In an embodiment, products made with the water-soluble formulation of the present disclosure are sparged after the water-soluble formulation is added. In an embodiment, the products are sparged within at least 60 minutes, at least 45 minutes, at least 30 minutes, at least 10 minutes, at least 5 minutes or less, after the water-soluble formulation is added. In an embodiment, the sparged products are also treated with reducing agents.

The water-soluble formulation and/or the products containing the water-soluble formulations may be treated with agents to adjust the pH. In an embodiment, the pH of the water-soluble formulation and/or the products containing the water-soluble formulation is adjusted to less than 4.5. In an embodiment, the pH of the water-soluble formulation and/or the products containing the water-soluble formulation is adjusted to between 2.5 and 4.5. In an embodiment, the pH of the water-soluble formulation and/or the products containing the water-soluble formulation is adjusted to between 3.8 and 4.3. In an embodiment, the water-soluble formulation and/or products containing the water-soluble formulation may contain buffers to maintain a constant pH.

Additives

In some embodiments, the water-soluble formulations or products (e.g. beverages, foodstuffs, etc.) of the present disclosure may further include additives, such as for example and without limitation terpenes, terpenoids, flavonoids, or any combination thereof. Such additives may be used to enhance flavour, viscosity, aroma and the like.

In an embodiment, the additives may be derived from cannabis plants. In an embodiment, the additives may be derived from natural sources other than a cannabis plant, such as a plant of a different species. Alternatively, in some embodiments, the additives may be synthetic or semi-synthetic compounds.

Terpenes and Terpenoids

In an embodiment, the water-soluble formulations herein may comprise one or more terpenes and/or terpenoids. In an embodiment, the product containing the water-soluble formulations may comprise one or more terpenes and/or terpenoids.

Within the context of this disclosure, the term “terpene” includes cannabis derived terpenes and non-cannabis derived terpenes.

Terpenes are a large and diverse class of organic compounds, produced by a variety of plants, particularly conifers, and by some insects such as termites or swallowtail butterflies, which emit terpenes from their osmeteria. Terpenes are also major constituents of Cannabis sativa plants. They often have a strong odor and may protect the plants that produce them by deterring herbivores and by attracting predators and parasites of herbivores. The difference between terpenes and terpenoids is that terpenes are hydrocarbons, whereas terpenoids contain additional functional groups.

They are the major components of resin, and of turpentine produced from resin. The name “terpene” is derived from the word “turpentine”. In addition to their roles as end-products in many organisms, terpenes are major biosynthetic building blocks within nearly every living creature. Steroids, for example, are derivatives of the triterpene squalene.

When terpenes are modified chemically, such as by oxidation or rearrangement of the carbon skeleton, the resulting compounds are generally referred to as terpenoids. Some authors will use the term terpene to include all terpenoids. Terpenoids are also known as isoprenoids.

Within the context of this disclosure, the term “terpene” includes hemiterpenes, monoterpenols, terpene esters, diterpenes, monoterpenes, polyterpenes, tetraterpenes, terpenoid oxides, sesterterpenes, sesquiterpenes, norisoprenoids, or their derivatives, as well as isomeric, enantiomeric, or optically active derivatives.

Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids. These derivatives are encompassed herein by the term “terpene”, unless specifically stated otherwise.

Within the context of this disclosure, the term terpene includes the α-(alpha), β-(beta), γ-(gamma), oxo-, isomers, or any combinations thereof.

Terpenes are the primary constituents of the essential oils of many types of plants and flowers. Essential oils are used widely as fragrances in perfumery, and in medicine and alternative medicines such as aromatherapy. Synthetic variations and derivatives of natural terpenes also greatly expand the variety of aromas used in perfumery and flavours used in food additives.

Higher amounts of terpenes are released by trees in warmer weather, acting as a natural form of cloud seeding. The clouds reflect sunlight, allowing the forest to regulate its temperature. The aroma and flavour of hops comes, in part, from sesquiterpenes (mainly alpha-humulene and beta-caryophyllene), which affect beer quality. Accordingly, in some embodiments, the water-soluble formulations of the present disclosure include hop-derived terpenes such as hop-derived terpene blends available as Aramis, Brewer's Gold, Bravo and the like, and combinations thereof.

Plant terpenes are used extensively for their aromatic qualities and play a role in traditional herbal remedies. Terpenes contribute to the scent of eucalyptus, the flavours of cinnamon, cloves, and ginger, the yellow colour in sunflowers, and the red colour in tomatoes.

Non-limiting examples of terpenes within the context of this disclosure include: 7,8-dihydro-alpha-ionone, 7,8-dihydro-beta-ionone, Acetanisole, Acetic Acid, Acetyl Cedrene, Anethole, Anisole, Benzaldehyde, Bergamotene (Alpha-cis-Bergamotene) (Alpha-trans-Bergamotene), Bisabolol (Beta-Bisabolol), Alpha Bisabolol, Borneo!, Bornyl Acetate, Butanoic/Butyric Acid, Cadinene (Alpha-Cadinene) (Gamma-Cadinene), Cafestol, Caffeic acid, Camphene, Camphor, Capsaicin, Carene (Delta-3-Carene), Carotene, Carvacrol, Dextro-Carvone, Laevo-Carvone, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/lcosane, Elemene (Beta-Elemene), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), lonol (3-oxo-alpha-ionol) (Beta-lonol), Ionone (Alpha-Ionone) (Beta-Ionone), 1psdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In select embodiments, the water-soluble formulations disclosed herein comprise a terpene selected from β-caryophyllene, caryophyllene oxide, borneol, 1,8-cineole, camphene, humulene (e.g., α-humulene), limonene (e.g., D-limonene, L-limonene), linalool, hexyl acetate, myrcene (e.g., 3-myrcene), nerolidol, pulegone, isopulegol, α-pinene, 3-pinene, para-cymene, eugenol, farnesol, geraniol, phytol, terpinene (e.g., gamma-terpinene), terpineol (e.g., α-terpineol) and terpinolene, or any combination thereof.

In particularly suitable embodiments, the water-soluble formulations include terpenes and/or terpenoids having antimicrobial properties. Exemplary antimicrobial terpenes include, for example, Ocimum basilicum (basil), Laurus nobilis (bay), Cinnamomum verum (Ceylon cinnamon), Capsicum annuum (paprika), Syzygium aromaticum (clove), Mentha piperita (peppermint), Tanacetum vulgare (tansy), Artemisia dracunculus (Tarragon), and the like as known in the art. This allows for a more stable water-soluble formulation. Particularly, it has been found that the water-soluble formulations show no bacterial growth for a time period of up to 50 days or more, even without the use of a preservative.

Furthermore, in some embodiments, the water-soluble formulations include a total terpene concentration (relative to the concentration of cannabinoids) beyond what would normally be found in the cannabis flower, allowing for the potential of entourage effects. As used herein, the “entourage-effect” refers to the residual effect of one or more compounds (e.g., cannabinoids, terpenes) of the water-soluble formulations in the sequentially administered end products including the water-soluble formulations.

Exemplary terpene blends for use in the water-soluble formulations are provided below.

Exemplary Terpene Blends:

| | | Beta- | | Hexyl | | Beta- |
|---|---|---|---|---|---|---|
| | | Pinene | Limonene | Acetate | Terpinolene | Caryophyllene |
| Strain | Formulation | % (w/w) | % (w/w) | % (w/w) | % (w/w) | % (w/w) |
| Penelope | 1 | 0.0 | 0.0 | 30.0 | 40.0 | 30.0 |
| | 2 | 0.0 | 20.0 | 10.0 | 40.0 | 30.0 |
| | 3 | 0.0 | 25.0 | 5.0 | 40.0 | 30.0 |
| | 4 | 0.0 | 25.0 | 5.0 | 30.0 | 40.0 |
| | 5 | 0.0 | 27.5 | 2.5 | 40.0 | 30.0 |
| | 6 | 0.0 | 20.0 | 2.5 | 40.0 | 37.5 |
| | 7 | 10.0 | 20.0 | 2.5 | 40.0 | 27.5 |
| | 8 | 10.0 | 30.0 | 2.5 | 30.0 | 27.5 |

| | | | | | | Beta- | |
|---|---|---|---|---|---|---|---|
| | | Eugenol | p-Cymeme | Humulene | Terpinolene | Caryophyllene | Limonene |
| Strain | Formulation | % (w/w) | % (w/w) | % (w/w) | % (w/w) | % (w/w) | % (w/w) |
| Houndstooth | 1 | 20.0 | 10.0 | 10.0 | 30.0 | 30.0 | |
| | 2 | 5.0 | 10.0 | 15.0 | 40.0 | 30.0 | |
| | 3 | 5.0 | 10.0 | 15.0 | 20.0 | 30.0 | 20.0 |
| | 4 | 5.0 | 20.0 | 15.0 | 20.0 | 10.0 | 30.0 |

| | | B-pinene | Nerolidol | Isopulegol | y-Terpinene |
|---|---|---|---|---|---|
| | | % (of terpene | % (of terpene | % (of terpene | % (of terpene |
| Strain | Formulation | total weight) | total weight) | total weight) | total weight) |
| Houndstooth | 1 | 40.0 | 20.0 | 20.0 | 20.0 |
| | 2 | 70.0 | 10.0 | 10.0 | 10.0 |
| | 3 | 60.0 | 0.0 | 20.0 | 20.0 |
| | 4 | 10.0 | 10.0 | 40.0 | 40.0 |
| | 5 | 20.0 | 40.0 | 0.0 | 40.0 |
| | 6 | 30.0 | 35.0 | 0.0 | 35.0 |

Flavonoids

In some embodiments, the water-soluble formulations may further include additives such as one or more flavonoids.

As used herein, the term “flavonoid” refers to any compound of a large class of plant pigments having a structure based on or similar to that of flavone. Chemically, flavonoids have the general structure of a 15-carbon skeleton, which consists of two phenyl rings and a heterocyclic ring.

Within the context of this disclosure, the term “flavonoids” includes bioflavonoids, isoflavonoids and neoflavonoids. Isoflavones use the 3-phenylchromen-4-one skeleton (with no hydroxyl group substitution on carbon at position 2). Examples include: Genistein, Daidzein, Glycitein, Isoflavanes, Isoflavandiols, Isoflavenes, Coumestans, and Pterocarpans.

Within the context of this disclosure, the term “flavonoids” also includes anthocyanidins, anthoxanthins, flavanones, flavanonols and flavens.

Flavonoids are widely distributed in plants, fulfilling many functions. Flavonoids are the most important plant pigments for flower colouration, producing yellow or red/blue pigmentation in petals designed to attract pollinator animals. In higher plants, flavonoids are involved in UV filtration, symbiotic nitrogen fixation and floral pigmentation. They may also act as chemical messengers, physiological regulators, and cell cycle inhibitors. Some flavonoids have inhibitory activity against organisms that cause plant diseases, e.g. Fusarium oxysporum.

Sources of flavonoids include, without limitation, cannabis, parsley, blueberries, black tea, citrus, wine, cocoa and peanut.

Additional exemplary flavonoids for use in the formulations include Apigenin, beta-sitosterol, cannaflavin A, kaempferol, luteolin, orientin, quercetin and combinations thereof.

In an embodiment, the flavonoid is cannaflavin.

Other Additives

In some embodiments, the water-soluble formulations or products (e.g. beverages, foodstuffs, etc.) of the present disclosure may include any number of other additives, including without limitation a solvent, a bulking agent, an antioxidant, or a nutritional supplement. These components may be used either alone or in combination to improve, for example, the chemical and/or physical properties, stability, nutritional profile, taste, colour and/or viscosity, of the water-soluble formulations disclosed herein or a beverage or foodstuff produced therefrom. In an embodiment, the antioxidant may be ascorbyl palmitate or α-tocopherol.

Yet other suitable types of modifiers and additives that may be used in the water-soluble formulations or products (e.g. beverages, foodstuffs, etc.) disclosed herein include viscosity modifiers, natural emulsifiers, oils, thickening agents, minerals, acids, bases, vitamins, flavours, colourants, sweeteners (e.g. liquid sweeteners), and the like and combinations thereof, as known in the beverage and food arts, to provide improved solubility, stability, bioavailability, colour and taste.

Nutritional supplements comprise substances useful to the consumer of the formulations disclosed herein, or beverages or foodstuffs prepared therewith, for maintenance of normal body health. Suitable nutritional supplements may comprise, for example, essential nutrients including vitamins, dietary minerals, amino acids and fatty acids. Exemplary nutritional supplements may include vitamin A, vitamin B1, vitamin B2, vitamin B3, vitamin B5, vitamin B6, vitamin B7, vitamin B9, vitamin B12, vitamin C, vitamin D, vitamin E, vitamin K calcium, phosphorus, potassium, sulfur, sodium, chlorine, magnesium, iron, cobalt, copper, zinc, molybdenum, iodine, selenium, manganese, nickel, chromium, fluorine, boron, strontium histidine, isoleucine, leucine, lysine, methionine, cysteine, phenylalanine, tyrosine, threonine, tryptophan, valine, alpha-linoleic acid, and linoleic acid.

Viscosity modifiers include any compound or agent capable of altering the viscosity of the formulations disclosed herein, or a beverage or foodstuff produced therewith. Exemplary embodiments of viscosity modifiers include anticaking agents, antifoaming agents, bulking agents, coagulation agents, gelling agents, glazing agents, humectants, leavening agents, tenderizers, and thickeners. In an embodiment, the viscosity modifying agent may be an unmodified starch, pregelatinized starch, cross-linked starches, gums (e.g. guar gum, xanthum gum, acacia), polyvinyl pyrrolidone (PVP), polyethylene oxide, waxes (e.g. beeswax), and mixtures thereof.

Sweeteners include any compound or agent that is capable of sweetening the taste of the formulations disclosed herein, or a beverage or foodstuff produced therewith. The sweetener may be a natural sweetener or an artificial sweetener. The sweetener may be a solid, liquid or semi-liquid. Exemplary embodiments of sweeteners include sugars and sugar alcohols, and more particularly stevia, erythritol, and xylitol. In an embodiment, the sweetener may be a liquid sugar solution, such as without limitation those having a Brix value of about 67.5 ° Bx. In an embodiment, the sweetener may be an Isocane 67.5 ° Bx liquid sugar solution.

In particularly suitable embodiments, the water-soluble formulations of the present disclosure are shelf-stable. As used herein, “shelf-stable” refers to the formulation maintaining a homogeneous mixture (i.e., no phase separation) for a period of at least 30 days, more suitably, at least 40 days, even more suitably, at least 45 days, and more suitably, at least 50 days, and even more suitably, at least 55 days or longer.

In particularly suitable embodiments, the water-soluble formulations of the present disclosure enhance or maintain the stability of the cannabinoids or cannabis-derived compounds in the water-soluble formulation, in a product produced therefrom (e.g. beverage), or both. In an embodiment, loss of cannabinoids or cannabis-derived compounds in the water-soluble formulations of the present disclosure is less than 35% by weight in 3 months, more particularly less than 25% by weight in 3 months, and more particularly still less than 20% by weight in 3 months. In an embodiment, loss of cannabinoids or cannabis-derived compounds in the water-soluble formulations of the present disclosure is about 25%, about 20%, about 15%, about 10%, about 5%, or less, by weight in 3 months. In an embodiment, loss of cannabinoids or cannabis-derived compounds in the water-soluble formulations of the present disclosure is less than 16% by weight THC content in 70 days.

In particularly suitable embodiments, the water-soluble formulations of the present disclosure, when mixed with an aqueous solution, provide a product which is stable. By “stable”, it is meant that the water-soluble formulation remains free from one or more deleterious changes over a period of time, for example at least or longer than 1 day, 1 week, 1 month, 3 months, 6 months, 1 year, or more. For example, stable may be in reference to a lack of degradation of cannabinoids or cannabis-derived compounds; a maintenance of clarity; or a maintenance of any other property desirable for consumption.

In an embodiment, the water-soluble formulation, when mixed with an aqueous solution, provides a product in which at least 60%, at least 65%, at least 70%, at least 75%, at least 80%, at least 85%, at least 90%, or at least 95% by weight of the cannabinoid or cannabis-derived compound remains present after about 2 months at a temperature between about 17° C. and about 40° C. In an embodiment, at least 80% by weight of the cannabinoid or cannabis-derived compound remains present after about 2 months at a temperature between about 17° C. and about 40° C. In an embodiment, at least 90% by weight of the cannabinoid or cannabis-derived compound remains present after about 2 months at a temperature between about 17° C. and about 40° C.

In an embodiment, the water-soluble formulation, when mixed with an aqueous solution, provides a product in which at least 60%, at least 65%, at least 70%, at least 75%, at least 80%, at least 85%, at least 90%, or at least 95% by weight of the cannabinoid or cannabis-derived compound remains present after about 3 months at a temperature between about 17° C. and about 40° C. In an embodiment, at least 80% by weight of the cannabinoid or cannabis-derived compound remains present after about 3 months at a temperature between about 17° C. and about 40° C. In an embodiment, at least 90% by weight of the cannabinoid or cannabis-derived compound remains present after about 3 months at a temperature between about 17° C. and about 40° C.

In an embodiment, at least 80% by weight of the cannabinoid or cannabis-derived compound remains present after about 2 months at about 40° C. In an embodiment, at least 90% by weight of the cannabinoid or cannabis-derived compound remains present after about 2 months at about 40° C.

In an embodiment, at least 80% by weight of the cannabinoid or cannabis-derived compound remains present after about 3 months at about 40° C. In an embodiment, at least 90% by weight of the cannabinoid or cannabis-derived compound remains present after about 3 months at about 40° C.

In an embodiment, at least 84.89% by weight of the cannabinoid or cannabis-derived compound remains present after about 3 months at a temperature of about 40° C.

In particularly suitable embodiments, the water-soluble formulations of the present disclosure are substantially free of cyclodextrins and modified starches, thereby reducing unnatural ingredients from end use products including the formulations.

Additionally, the water-soluble formulations of the present disclosure are suitably prepared to be low calorie. Particularly, in some embodiments, a 250 mL serving will provide less than 25 kilocalories (Kcal), more suitably less than 10 Kcal, and even more suitably less than 5 Kcal.

Methods o Preparing the Water-Soluble Formulations

It has been found that by mixing the above-described components of the water solution formulations in a particular order, a cloudy pre-emulsion can be formed that, when mixed with an aqueous solution, can form a transparent or translucent microemulsion having favorable pharmacokinetics, for example, rapid onset, shorter duration, and minimal food effect. In select embodiments, the water-soluble formulations produced by the disclosed methods may also be clear, rather than cloudy.

In one aspect, to prepare the water-soluble formulations of the present disclosure, a cannabinoid or a cannabis-derived compound, a glycerin-based carrier surfactant and an emulsifier are mixed, in any order.

In select embodiments, the water-soluble formulations comprise a carrier oil. Accordingly, in another aspect, to prepare the water-soluble formulations of the present disclosure, the cannabinoid or cannabis-derived compound (e.g., cannabinoid distillate and/or isolate) and the carrier oil are first mixed to form a homogenous mixture; and then the surfactant (e.g. glycerin-based carrier surfactant) and emulsifier are mixed into the homogenous mixture.

In a particular embodiment, the method comprises: mixing a cannabinoid or a cannabis-derived compound and a carrier oil until a homogenous mixture is formed; and mixing a glycerin-based carrier surfactant and an emulsifier into the homogenous mixture to prepare the water-soluble formulation. In an embodiment, the cannabis-derived compound is a cannabinoid distillate or isolate; the carrier oil is monoglycerides; the emulsifier is a soy lecithin; and the glycerin-based carrier surfactant is a vegetable glycerin.

In a particular embodiment, the method comprises: mixing a cannabinoid or a cannabis-derived compound with a carrier oil to provide a cannabinoid mixture; mixing a glycerin-based carrier surfactant and an emulsifier comprising Quillaja saponins to provide a glycerin-based mixture; and combining the cannabinoid mixture and the glycerin-based mixture under high shear homogenization to prepare the water-soluble formulation. In an embodiment, the cannabis-derived compound is a cannabinoid distillate or isolate; the carrier oil is monoglycerides or a medium-chain triglycerides; and the glycerin-based carrier surfactant is glycerin.

During the step of mixing the cannabinoid or cannabis-derived compound with the carrier oil, heat may be applied. In an embodiment, the mixing is performed under heated conditions of between about 40° C. and about 75° C.

As discussed herein, the water-soluble formulation may comprise more than one emulsifier. When two or more emulsifiers are used, they may be mixed into the homogenous mixture together or in any order, including consecutively or simultaneously. In an embodiment a single emulsifier is used, such as a soy lecithin. In an embodiment, two emulsifiers are used such as a soy lecithin and a sucrose monoester.

When a carrier oil is used, the cannabinoid or cannabis-derived compound (e.g. cannabinoid distillate and/or isolate) and carrier oil may be mixed using any methods known in the art to reduce the size of the cannabinoid particles in the oil to form a homogenous mixture. Suitable methods include, for example, homogenization methods as known in the art (e.g., high-pressure homogenization (H PH), high-shear homogenization, microfluidization). In some embodiments, the cannabinoid or cannabis-derived compound and carrier oil are mixed under heated conditions such as by mixing in a microwave.

Small droplet sizes lead to transparent emulsions. In an embodiment, droplet sizes of between about 30 nm and about 100 nm are desirable for the homogenous mixture. In an embodiment, droplet sizes about 100, 90, 80, 70, 60, 50 or 40 nm are desirable for the homogenous mixture. Suitably, the droplet sizes for homogenous emulsions are in the range of 40 to 60 nm, more suitably they are 45 to 55 nm, more suitably yet, 50 nm.

Once the homogenous mixture is formed, the surfactant and emulsifier can be mixed into the homogenous mixture. Suitable methods for mixing the surfactant and emulsifier into the homogenous mixture include any known methods for mixing components into a formulation. It has been found, however, that by first adding the surfactant to the homogenous mixture, yet not mixing in the surfactant, with the mixture and then subsequently slowing adding the emulsifier to the surfactant-containing mixture, gelatinous clumps can be avoided. In embodiments in which more than one emulsifier is used, they may be added separately at different times, added separately at the same time, or mixed together and then added together. In a particular embodiment, two emulsifiers are added separately at the same time.

This mixture can then be homogenized, such as by using a bench top homogenizer to mix all the ingredients thoroughly. After this, the mix can be put into a microfluidizer where between about 2,500 and about 40,000 psi, more particularly between about 10,000 and about 40,000 psi, and more particularly still between about 20,000 and about 40,000 psi, of pressure is applied to create an emulsion system with very small particles (<100 nm). In an embodiment, the microfluidizing provides a particle size of about 40 nm. In an embodiment, the resulting water-soluble formulation is completely clear. In an embodiment, using a high shear homogenizer is sufficient to provide the homogenized mixture without the need of a microfluidizer.

Further, if any additives as described above are to be included in the water-soluble formulation, it is suitable to mix the additives into the homogenous mixture prior to mixing the surfactant and emulsifier into the homogenous mixture.

A product may be prepared by mixing the water-soluble formulation with an aqueous solution. In an embodiment, the product is a beverage. In an embodiment, the product is a sports drink beverage. In an embodiment, the method further comprises mixing a sucrose monoester into the homogenous mixture.

Further, if any additives as described herein are to be included in the product, it is suitable to mix the additives into the product at the appropriate stage. For example, in an embodiment, the product may comprise one or more of: terpenes, terpenoids, flavonoids, viscosity modifiers, natural emulsifiers, oils, thickening agents, minerals, acids, bases, vitamins, flavours, colourants, sweeteners, and combinations thereof. In a particular embodiment, the method further comprises mixing a chelating agent into the aqueous solution. In an embodiment, the chelating agent is EDTA.

A solid product may also be prepared. An exemplary method for preparing a solid product comprises: mixing a cannabinoid or a cannabis-derived compound and a carrier oil until a homogenous mixture is formed, as described above; mixing the surfactant and emulsifier into the homogenous mixture to prepare the water-soluble formulation as described above; and absorbing the water-soluble cannabis formulation into or onto a solid material (e.g. tea bag).

Products Including the Water-Soluble Formulations

The present disclosure is further directed to using the water-soluble formulations to form end use products such as ingestibles, topical solids and liquids. The ingestibles can include, for example, beverages, liquids and foodstuffs.

Thus, the water-soluble formulations of the present disclosure may be used in the preparation of foodstuffs and beverages. As used herein, a beverage is any drink that may be consumed by a subject. A foodstuff is any substance suitable for consumption as a food.

The compositions may be combined with any beverage-compatible or food-compatible ingredient. For example, water-soluble formulations of the present disclosure may be used directly in the preparation of foodstuffs and beverages, e.g. as an additive or ingredient. Powder formulations may be used either directly, e.g. as an additive or ingredient, or indirectly e.g. by first dissolving the powder in a solvent (e.g. water) to form a liquid composition prior to use. In some embodiments, the powder compositions may be added to beverage or foodstuff directly. In other embodiments, the powder formulations are diluted with a bulking agent. The pre-bulked and/or bulked powder compositions can be packaged for individual servings (e.g. sachets/packets), packages in bulk within a single container, or a combination thereof.

When used in beverages, the water-soluble formulations of the present disclosure further comprise a beverage liquid. Generally, beverage liquids are liquids meeting the common meaning of the term “biocompatible”, which include materials that are not harmful to living tissue. Suitably, such beverage liquids comprise water, oil, alcohol; with or without additives or modifiers or both. Such beverage liquids can be divided into various groups such as plain/still water, carbonated water, alcohol, non-alcoholic drink, soft drinks, electrolyte drinks (e.g. sports drink beverages), fruit juice, vegetable juice, tea, coffee, milk, or other hot, room temperature or cold liquids used in drinks. Beverages can be caffeinated or non-caffeinated and may contain calories or not. Such beverages may be produced in ready to use form or be produced in a form suitable for preparation in final consumable form at or proximate to the time of ingestion.

Typically, beverage liquids will make up between about 50% and 99.99% by weight or by volume of the beverage. In an embodiment, beverage liquids will make up between about 60% and about 99.99% by weight or by volume of the beverage, more particularly between about 70% and about 99.99% by weight or by volume of the beverage, more particularly still between about 80% to about 99.99% by weight or by volume of the beverage, and even more particularly still between about 80% to about 99.99% by weight or by volume of the beverage. In an embodiment, the beverage liquid will make up between about 80% and about 99.99% by weight of the beverage. In an embodiment, the beverage liquid will make up between about 80% and about 99.9% by weight of the beverage, between about 80% and about 99%, or between about 80% and about 90% by weight of the beverage. In an embodiment, the beverage liquid will make up between about 95% and about 99.9% by weight of the beverage. In an embodiment, the beverage liquid will make up about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more by weight of the beverage. In an embodiment, the beverage liquid will make up about 99.0%, about 99.1%, about 99.2%, about 99.3%, about 99.4%, about 99.5%, about 99.6%, about 99.7%, about 99.8%, or about 99.9% by weight of the beverage. In an embodiment, the beverage liquid is water. In an embodiment, additives may be present in addition to the quantity of beverage liquid. In an embodiment, a liquid additive (e.g. sweetener) may be present in addition to the quantity of beverage liquid.

In a particular embodiment, the beverage liquid will make up between about 80% and about 95% by weight of the beverage, and a liquid additive (e.g. sweetener) will make up between about 4.9% and about 14.9% by weight of the beverage. In select embodiments, the beverage liquid will make up about 80%, about 81%, about 82%, about 83%, about 84%, about 85%, about 86%, about 87%, about 88%, about 89%, about 90%, about 91%, about 92%, about 93%, about 94%, about 95% by weight of the beverage. In select embodiments, a liquid additive (e.g. sweetener) will make up about 18%, about 17%, about 16%, about 15%, about 14%, about 13%, about 12%, about 11%, about 10%, about 9%, about 8%, about 7%, about 6%, about 5%, about 4%, about 3% or less by weight of the beverage. In an embodiment, the beverage liquid is water and the liquid additive is a liquid sweetener, such as for example a 67.5 ° Bx (Brix) sugar solution.

Non-limiting examples of beverages that may be prepared with the water-soluble formulations of the present disclosure include but are not limited to: hot and cold beverages including water, fruit juice, vegetable juice, tea, coffee, softs drinks, energy drinks, alcohol, flavoured water, or single-serve beverage cartridges. Non-limiting examples of foodstuffs include baked goods (e.g. cookies, brownies, cake, pie, biscuits and pastries), candies (e.g. hard candy, soft candy, gummies, etc.), chocolates, lozenges, gum, mints, dried fruits, nuts, granola, truffles, caramels, chews, taffy, prepared meals, cooking ingredients (e.g. food additives, dry spices, honey, sugar, sweeteners, etc.), ground coffee, instant coffee and tea leaves.

The amount of the water-soluble formulation of the present disclosure added to beverages or foodstuffs will vary depending on the desired dosage of cannabinoids (e.g. THC and CBD) or cannabis-derived compound. For example, in some embodiments each serving, unit or item of foodstuff or beverage will contain between about 0.5 mg and about 100 mg of cannabinoids. In an embodiment, the foodstuff or beverage will contain between about 1.0 mg and about 50 mg of cannabinoids or cannabis-derived compound, more particularly between about 1.0 mg and about 25 mg of cannabinoids, between about 2.0 mg and about 20 mg, or between about 2.0 mg and about 10 mg of cannabinoids or cannabis-derived compound. In an embodiment, the foodstuff or beverage will contain about 0.5 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 10.0 mg, about 10.5 mg, about 11.0 mg, about 11.5 mg, about 12.0 mg, about 12.5 mg, about 13.0 mg, about 13.5 mg, about 14.0 mg, about 14.5 mg, about 15.0 mg, about 15.5 mg, about 16.0 mg, about 16.5 mg, about 17.0 mg, about 17.5 mg, about 18.0 mg, about 18.5 mg, about 19.0 mg, about 19.5 mg, about 20.0 mg, about 20.5 mg, about 21.0 mg, about 21.5 mg, about 22.0 mg, about 22.5 mg, about 23.0 mg, about 23.5 mg, about 24.0 mg, about 24.5 mg, or about 25.0 mg of cannabinoids or cannabis-derived compound. The cannabinoids may be one type of cannabinoid (e.g. CBD or THC) or any combination of cannabinoids. In an embodiment, the cannabinoid is THC. In an embodiment, the cannabinoid is CBD.

In an embodiment, the foodstuff or beverage may contain a cannabinoid or cannabis-derived compound concentration of between about 0.01 mg/mL and about 2.5 mg/mL, more particularly between about 0.025 mg/mL and about 1.25 mg/mL, between about 0.025 mg/mL and about 1.0 mg/mL, or between about 0.05 mg/mL and about 0.1 mg/mL. In an embodiment, the foodstuff or beverage may contain a cannabinoid or cannabis-derived compound concentration of about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.25 mg/mL, about 1.5 mg/mL, about 1.75 mg/mL, about 2.0 mg/mL, about 2.25 mg/mL, or about 2.5 mg/mL. The cannabinoid may be one type of cannabinoid (e.g. CBD or THC) or any combination of cannabinoids. In an embodiment, the cannabinoid is THC. In an embodiment, the cannabinoid is CBD.

In an embodiment, the product (e.g. beverage or foodstuff) may comprise between about 0.1% and about 25% by weight, particularly about 0.5% and about 25% by weight of the water-soluble formulation, more particularly between about 1% and about 10% by weight of the water-soluble formulation, and more particularly still between about 1% and about 5% by weight of the water-soluble formulation. In an embodiment, the product may comprise between about 0.5% and about 3% by weight of the water-soluble formulation. In some embodiments, low quantities by weight of the water-soluble formulation may be used due to advantageous properties of the water-soluble formulations of the present disclosure in stably formulating cannabinoids or cannabis-based compounds.

In one embodiment, the water-soluble formulations are mixed with an aqueous solution to prepare an end use product (e.g. beverage). The aqueous solution can include pure water alone, or an aqueous solution including water and additives such as the additives described above to improve end use product stability, bioavailability, colour, aroma and taste. Particularly, additives may include terpenes, terpenoids, flavonoids, viscosity modifiers, natural emulsifiers, oils, thickening agents, minerals, acids, bases, vitamins, flavours, colourants, sweeteners (liquid and/or solid), and the like and combinations thereof, as known in the beverage and food arts.

In an embodiment, the aqueous solution comprises a stabilizer as described herein. In an embodiment, the aqueous solution comprises a chelating agent. The chelating agent may be added before or after the water-soluble formulation is mixed with the aqueous solution. In an embodiment, the chelating agent is EDTA.

In some embodiments, the end products incorporating the water-soluble formulations of the present disclosure include less than 20% by weight glycerin, including less than less than 15% by weight, less than 10% by weight, less than 5% by weight, and less than 1% by weight glycerin.

Beverages may be are packaged as individual packages, suitably single use packages, and multiple packages. The packaging can be in air tight containers. Packaging may be comprised of paper, plastic, metal, and glass. In an embodiment, the packaging may be Tetra Pak™0 packaging. Beverages may include bubble containing or producing liquids with dissolved gas or liquids capable of producing gas proximately in time of consumption. In one embodiment of the disclosure, the beverages, optionally comprising additives, modifiers or both, are convenient to consumers, and are manufactured at modest expense. Beverages with dissolved gas may be created by a method comprising addition of carbon dioxide, ozone, oxygen, and nitrogen. For beverages with dissolved gas, dissolved gas may be added to the beverage by methods comprising application of pressure, and adding water with the dissolved gas. The dissolved gas is released from the beverage when pressure is reduced as effervescence.

In another embodiment, the water-soluble formulations are absorbed into a solid material for use as an end use product. By way of example, the water-soluble formulations may be absorbed onto one or more of blotter paper, tea leaves, coffee grounds, spices and the like to allow for a convenient water-soluble edible or tea bag.

The compositions of the present disclosure are suitably low calorie, and can be used to prepare beverages and foodstuffs that are low calorie. Particularly, in some embodiments, a 250 mL or 2-5 g serving will provide less than 25 kilocalories (Kcal), more suitably less than 10 Kcal, and even more suitably less than 5 Kcal.

In some further embodiments, the water-soluble formulations are further dried to form a powder formulation for use in liquid beverages and foods. The above described formulations may be dried using any method as known in the drying arts to evaporate the water phase of the emulsion, and possibly none, some or essentially all of the carrier solvent. For example, in one embodiment, the formulations are spray dried to form the powder formulation. Alternative methods of preparing the dried powder formulation include, but are not limited to, pan coating, air-suspension coating, centrifugal extrusion, vibrational nozzle technique, freeze-drying or using a food dehydrator.

In some embodiments, the powder formulation can be diluted with a bulking agent or a mixture of bulking agents. Suitable bulking agents include, for example, gum arabic, waxy maize starch, dextrin, maltodextrin, polydextrose, inulin, fructooligosaccharide, sucrose, glucose, fructose, galactose, lactose, maltose, trehalose, cellobiose, lactulose, ribose, arabinose, xylose, lyxose, allose, altrose, mannose, gulose, talose, erythritol, threitol, arabitol, xylitol, mannitol, ribitol, galactitol, fucitol, inositol, maltitol, sorbitol, isomalt, lactitol, polyglycitol, iditol, volemitol, maltotriitol, maltotetraitol, maltol, stevia, stevio side, rebaudio side, neotame, sucralose, saccharin, sodium cyclamate, aspartame, acesulfame potassium, chitin, and chitosan. In an embodiment, the bulking agent is erythritol. In an embodiment, the bulking agent is sucrose. In an embodiment, the bulking agent is inositol. In an embodiment, the bulking agent is myo-inositol.

In some aspects, the bulking material may comprise a sweetener, pH modifier, pH stabilizer, antimicrobial preservative, antioxidant, texture modifier, colourant or combinations thereof.

In some embodiments, the bulked powder formulations comprise at least 0.001% by weight, and suitable from 0.001% by weight to about 3% by weight, of a cannabinoid or a cannabis-derived compound. More suitably, a dosage form for an exemplary product includes 10 milligram of tetrahydrocannabinol (THC) per serving. Assuming a 3.5 gram serving size, the bulk powder formulation would contain approximately 0.3% by weight of the primary cannabinoid (e.g. THC and/or CBD). Assuming a 5 gram sample size, the bulk powder formulation would contain approximately 0.2% by weight of the primary cannabinoid.

Once prepared, the powder formulation may be mixed directly in a liquid beverage or food or may first be dissolved in a solution to then be added to a liquid beverage or food. The powder formulation may be prepared and packaged using any packaging known in the art. For example, in one embodiment, the powder formulation may packaged as a single serving or multiple servings in a metal, glass, or plastic container. In another embodiment, the powder formulation may be packaged as a single serving stick pack.

Thus, in various embodiments, the present disclosure relates to a product comprising and/or produced using the water-soluble formulation described herein. In an embodiment, the product is a beverage further comprising an aqueous solution. In an embodiment, the product comprises a cannabinoid distillate or a cannabinoid isolate; monoglycerides; a soy lecithin; a sucrose monoester; and a vegetable glycerin. In an embodiment, the beverage comprises a stabilizer, such as for example any stabilizer described herein and for example a chelating agent.

In some embodiments, the water-soluble formulations, beverages and/or foodstuffs disclosed herein provide a desired intoxication effect as measured by a standard British unit of alcohol. As used herein, “one British unit of alcohol” is defined as 10 mL (8 g) of pure alcohol. That is the number of units of alcohol can be determined by multiplying the volume of the drink (in milliliters) by percentage ABV, and dividing by 1000.

Suitably, in some aspects, the beverages or foodstuffs are formed and administered to provide a subjective or objective intoxicating effect equivalent to a standard British unit of alcohol. More particularly, from about 25 mL to 500 mL of the beverage, more particularly, from about 35 mL to about 250 mL, and even more particularly, from about 60 mL to about 120 mL of the beverage, are formed and administered to provide an intoxicating effect equivalent to a standard British unit of alcohol. By further way of example, in one aspect, consuming about 35 mL to about 60 mL of the beverage causes either a subjective or objective intoxicating effect equivalent to a standard British unit of alcohol. In another aspect, consuming about 60 mL to about 120 mL of the beverage causes either a subjective or objective intoxicating effect equivalent to a standard British unit of alcohol. In yet another aspect, consuming about 120 mL to about 250 mL of the beverage causes either a subjective or objective intoxicating effect equivalent to a standard British unit of alcohol. In yet another aspect, consuming about 250 mL to about 500 mL of the beverage causes either a subjective or objective intoxicating effect equivalent to a standard British unit of alcohol.

It will further be appreciated that in certain embodiments the beverage or foodstuff should provide the human or non-human subject an intoxicating effect at the desired time. For example, in some embodiments, the beverage or foodstuff provides for an onset of intoxication in a time period of from about 10 minutes to about 120 minutes, including from about 20 minutes to about 90 minutes, and including from about 30 minutes to about 60 minutes, after consumption of the beverage or foodstuff. By way of further example, in certain embodiments the beverage or foodstuff can be formed and administered to provide for an onset of the intoxication of about 10 minutes, or about 15 minutes, or about 20 minutes, or about 25 minutes, or about 30 minutes, 40 minutes, 60 minutes, 90 minutes, or even 120 minutes. In further examples and embodiments, the beverage or foodstuff can be formed and administered to provide for an onset of the intoxication of about 180 minutes, or even about 240 minutes, or even still about 300 minutes.

Advantageously, embodiments of the products (e.g. beverages and/or foodstuffs) comprising or produced using the water-soluble formulations of the present disclosure are shelf-stable.

As used in the context of the products herein, “shelf-stable” refers to the water-soluble formulation maintaining its water-soluble nature in an aqueous product at least in respect of the cannabinoid or cannabis-derived compound (e.g., no precipitation of these compounds) for a period of at least 30 days, more suitably, at least 40 days, even more suitably, at least 45 days, and more suitably, at least 50 days, and even more suitably, at least 55 days or longer.

In particularly suitable embodiments, the products disclosed herein enhance or maintain the stability of the cannabinoids or cannabis-derived compounds. In an embodiment, loss of cannabinoids or cannabis-derived compounds in the products disclosed herein is less than 35% by weight in 3 months, more particularly less than 25% by weight in 3 months, and more particularly still less than 20% by weight in 3 months. In an embodiment, loss of cannabinoids or cannabis-derived compounds in the products disclosed herein is about 25%, about 20%, about 15%, about 10%, about 5%, or less, by weight in 3 months. In an embodiment, loss of cannabinoids or cannabis-derived compounds in the products disclosed herein is less than 16% by weight THC content in 70 days.

In particularly suitable embodiments, the products disclosed herein are stable. By “stable”, it is meant that the products remain free from one or more deleterious changes over a period of time, for example at least or longer than 1 day, 1 week, 1 month, 3 months, 6 months, 1 year, or more. For example, stable may be in reference to a lack of degradation of cannabinoids or cannabis-derived compounds; a maintenance of clarity; or a maintenance of any other property desirable for consumption.

In an embodiment, at least 60%, at least 65%, at least 70%, at least 75%, at least 80%, at least 85%, at least 90%, or at least 95% by weight of the original or time 0 quantity of the cannabinoid or cannabis-derived compound remains present in the product after about 2 months at a temperature between about 17° C. and about 40° C. In an embodiment, at least 80% by weight of the original or time 0 quantity of the cannabinoid or cannabis-derived compound remains present in the product after about 2 months at a temperature between about 17° C. and about 40° C. In an embodiment, at least 90% by weight of the original or time 0 quantity of the cannabinoid or cannabis-derived compound remains present in the product after about 2 months at a temperature between about 17° C. and about 40° C.

In an embodiment, at least 60%, at least 65%, at least 70%, at least 75%, at least 80%, at least 85%, at least 90%, or at least 95% by weight of the original or time 0 quantity of the cannabinoid or cannabis-derived compound remains present in the product after about 3 months at a temperature between about 17° C. and about 40° C. In an embodiment, at least 80% by weight of the original or time 0 quantity of the cannabinoid or cannabis-derived compound remains present in the product after about 3 months at a temperature between about 17° C. and about 40° C. In an embodiment, at least 90% by weight of the original or time 0 quantity of the cannabinoid or cannabis-derived compound remains present in the product after about 3 months at a temperature between about 17° C. and about 40° C.

In an embodiment, at least 80% by weight of the original or time 0 quantity of the cannabinoid or cannabis-derived compound remains present in the product after about 2 months at about 40° C. In an embodiment, at least 90% by weight of the original or time 0 quantity of the cannabinoid or cannabis-derived compound remains present in the product after about 2 months at about 40° C.

In an embodiment, at least 80% by weight of the original or time 0 quantity of the cannabinoid or cannabis-derived compound remains present in the product after about 3 months at about 40° C. In an embodiment, at least 90% by weight of the original or time 0 quantity of the cannabinoid or cannabis-derived compound remains present in the product after about 3 months at about 40° C.

In an embodiment, at least 84.89% by weight of the original or time 0 quantity of the cannabinoid or cannabis-derived compound remains present in the product after about 3 months at a temperature of about 40° C.

In any of the embodiments described herein, the product may have a reduced oxygen content, such as by removing the oxygen by means of equipment designed to perform this function or by chemical removal (e.g. N2 purge and/or potassium disulfite). In an embodiment, the oxygen content of the product is between about 0 ppm and about 500 ppm. In an embodiment, the product is sealed until use in order to maintain the reduced oxygen content.

Dosage Forms

A dosage form is that object delivered to a subject human or non-human organism for testing, placebo, recreational, therapeutic or other use. In an embodiment, the compositions of the present disclosure may be formulated as dosage forms for administration to a subject (e.g. the liquid or powder formulation within a soft gel capsule; a tablet comprising the powder formulation; the liquid or powder formulation absorbed onto or into a solid material).

Thus, in some embodiments, the dried powder formulation can be formulated into pharmaceutical dosage forms comprising an effective amount of particles. Although mainly pharmaceutical dosage forms for oral administration such as tablets and capsules are envisaged, the particles of the present disclosure can also be used to prepare pharmaceutical dosage forms e.g., for rectal administration. Preferred dosage forms are those adapted for oral administration shaped as a tablet. They can be produced by conventional tabletting techniques with conventional ingredients or excipients and with conventional tabletting machines.

As known in the art, tablet blends (including the powder formulations disclosed herein and any other conventional tablet ingredient or excipient) may be dry-granulated or wet-granulated before tabletting. The tabletting process itself is otherwise standard and readily practised by moulding a tablet from a desired blend or mixture of ingredients into the appropriate shape using a conventional tablet press.

Tablets may further be film-coated to improve taste or provide ease of swallowing and an elegant appearance. Many suitable polymeric film-coating materials are known in the art. A preferred film-coating material is hydroxypropyl methylcellulose HPMC, especially HPMC 2910 5 mPas. Other suitable film-forming polymers also may be used herein, including hydroxypropylcellulose and acrylate-methacrylate copolymers. Besides a film-forming polymer, the film coat may further comprise a plasticizer (e.g. propylene glycol) and, optionally, a pigment (e.g. titanium dioxide). The film-coating suspension also may contain talc as an anti-adhesive.

As noted above, embodiments of end use products including the water-soluble formulations of the present disclosure show improved pharmacokinetics, for example, rapid onset, shorter duration, consistent experience and minimal food effect.

Perceived onset is driven by total dosage consumed in one unit of time and how quickly the cannabinoids are absorbed after ingestion. With the use of the water-soluble formulations having a nanometer average particle size of components, increased surface area for absorption is achieved, allowing for improved onset. Further, the use of the carrier oils including the emulsified cannabinoids or cannabis-derived compounds, lymphatic absorption is encouraged, thereby bypassing first pass metabolism and food effects. Finally, the use of the biocompatible surfactant (e.g. glycerin-based carrier surfactant) in the water-soluble formulation increases uptake of the cannabinoids within the dosage forms by mimicking natural metabolic processes in the gut.

Consistency is driven by the stability of the water-soluble formulation used in the end use product and dosage form, which as described above is improved as compared to conventional cannabis formulations.

Suitable dosages of the end use products and dosage forms will depend upon many factors including, for example, age and weight of an individual, at least one precise event requiring professional consultation, severity of an event, specific water-soluble formulation to be used in the end product, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

In some embodiments, the end use products and dosage forms are prepared with water-soluble formulations in a dosage form and administration regime to provide a desired intoxication effect as measured by a standard British unit of alcohol, as described elsewhere herein in respect of products. This disclosure is equally applicable in respect of dosage forms.

### Exemplary Embodiments

The following are non-limiting and exemplary embodiments of the present disclosure:

(1) A water-soluble formulation comprising a cannabinoid or a cannabis-derived compound; an emulsifier; and a glycerin-based carrier surfactant.

(2) The water-soluble formulation of (1), further comprising a carrier oil.

(3) The water-soluble formulation of (2), wherein the carrier oil is comprised of monoglycerides.

(4) The water-soluble formulation of (3), wherein the monoglycerides comprise glyceryl monostearate, glyceryl hydroxystearate, glyceryl monoleate, winterized glyceryl monoleate, monolaurin, glyceryl monolinoleate, or any combination thereof.

(5) The water-soluble formulation of any one of (2) to (4), which comprises up to 10% by weight of the cannabinoid or cannabis-derived compound; up to 10% by weight of the carrier oil, and up to 10% by weight of the emulsifier.

(6) The water-soluble formulation of any one of (2) to (5), which comprises the cannabinoid or cannabis-derived compound; the carrier oil, and the emulsifier at an about equivalent amount by weight.

(7) The water-soluble formulation of any one of (1) to (6), which is an emulsion.

(8) The water-soluble formulation of any one of (1) to (7), which is clear.

(9) The water-soluble formulation of any one of (1) to (8), which is transparent, translucent, or pearlescent when mixed with an aqueous solution.

(10) The water-soluble formulation of any one of (1) to (9), wherein the cannabinoid is Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), Δ10-tetrahydrocannabinol (Δ10-THC), Tetrahydrocannabinolic acid C4 (THCA-C4), Tetrahydrocannbinol C4 (THC C4), Tetrahydrocannabivarinic acid (THCVA), Tetrahydrocannabivarin (THCV), Δ8-Tetrahydrocannabivarin (Δ8-THCV), Δ9 Tetrahydrocannabivarin (Δ9-THCV), Tetrahydrocannabiorcolic acid (THCA-C1), Tetrahydrocannabiorcol (THC-C1), Delta 7 cis iso tetrahydrocannabivarin, Δ8 tetrahydrocannabinolic acid (Δ8-THCA), Δ9 tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-ΔH-THC), 11 nor 9-carboxy-Δ9-tetrahydrocannabinol, Ethoxy-cannabitriolvarin (CBTVE), 10 Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-05), Dehydrocannabifuran (DCBF), Cannbifuran (CBF), Cannabichromanon (CBCN), Cannabicitran (CBT), 10 Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, Dodeca-2E,4E-dienoic acid isobutylamide, or any combination thereof.

(11) The water-soluble formulation of any one of (1) to (9), wherein the cannabinoid is cannabidiol (CBD), tetrahydrocannabinol (THC), or a combination thereof.

(12) The water-soluble formulation of any one of (1) to (9), wherein the cannabis-derived compound is a cannabis-derived cannabinoid, a cannabinoid distillate, a cannabinoid isolate, a terpene, or any combination thereof.

(13) The water-soluble formulation of any one of (1) to (12), wherein the emulsifier comprises a soy lecithin.

(14) The water-soluble formulation of any one of (1) to (12), wherein the emulsifier comprises a sucrose monoester.

(15) The water-soluble formulation of any one of (1) to (12), wherein the emulsifier comprises a soy lecithin and a sucrose monoester.

(16) The water-soluble formulation of (15), wherein the sucrose monoester is sucrose monopalmitate, sucrose monolaurate, sucrose monostearate, or any combination thereof.

(17) The water-soluble formulation of (16), wherein the sucrose monoester is sucrose monopalmitate.

(18) The water-soluble formulation of any one of (15) to (17), which comprises an about equivalent amount by weight of the soy lecithin and the sucrose monoester.

(19) The water-soluble formulation of any one of (1) to (18), wherein the glycerin-based carrier surfactant is a vegetable glycerin.

(20) The water-soluble formulation of any one of (1) to (19), which comprises between about 60% and about 97% by weight of the glycerin-based carrier surfactant.

(21) The water-soluble formulation of any one of (1) to (20), wherein the water-soluble formulation is shelf-stable at room temperature.

(22) The water-soluble formulation of (21), which is shelf-stable for at least 55 days.

(23) The water-soluble formulation of any one of (1) to (22), wherein the water-soluble formulation loses less than 20% by weight of the cannabinoid or cannabis-derived compound in 3 months.

(24) The water-soluble formulation of (23), which loses less than 16% by weight THC content in 70 days.

(25) The water-soluble formulation of any one of (1) to (24), wherein, when mixed with an aqueous solution, provides a product which is stable.

(26) The water-soluble formulation of any one of (1) to (25), wherein, when mixed with the aqueous solution, provides a product in which at least 80% by weight of the cannabinoid or cannabis-derived compound remains present after about 2 months at a temperature between about 17° C. and about 40° C.

(27) The water-soluble formulation of (26), wherein, at least 90% by weight of the cannabinoid or cannabis-derived compound remains present in the product after about 2 months at a temperature between about 17° C. and about 40° C.

(28) The water-soluble formulation of any one of (1) to (25), wherein, when mixed with the aqueous solution, provides a product in which at least 84.89% by weight of the cannabinoid or cannabis-derived compound remains present after about 3 months at a temperature of about 40° C.

(29) The water-soluble formulation of any one of (25) to (28), wherein the product has an oxygen content of between about 0 ppm and about 500 ppm.

(30) The water-soluble formulation of any one of (1) to (29), comprising less than 10 kcal per 250 mL of formulation.

(31) The water-soluble formulation of any one of (1) to (30), which is substantially free of cyclodextrins and modified starches.

(32) The water-soluble formulation of any one of (1) to (31) further comprising one or more additives selected from the group consisting of terpenes, terpenoids, flavonoids, viscosity modifiers, natural emulsifiers, oils, thickening agents, minerals, acids, bases, vitamins, flavours, colourants, and combinations thereof. \

(33) The water-soluble formulation of (32) comprising a terpene having antimicrobial properties.

(34) The water-soluble formulation of (32) or (33) comprising a hop-derived terpene blend selected from the group consisting of Aramis, Brewer's Gold, Bravo and combinations thereof.

(35) A powder formulation prepared by drying the water-soluble formulation of any one of (1) to (34).

(36) The powder formulation of (35) comprising less than 10 kcal per 250 mg of the powder formulation.

(37) A product comprising the water-soluble formulation of any one of (1) to (34).

(38) The product of (37), which is a beverage and further comprises an aqueous solution.

(39) The product of (37) or (38), comprising a cannabinoid distillate or a cannabinoid isolate; monoglycerides; a soy lecithin; a sucrose monoester; and a vegetable glycerin.

(40) The product of claim any one of (37) to (39), further comprising a stabilizer.

(41) The product of (40), wherein the stabilizer is a chelating agent.

(42) The product of (41), wherein the chelating agent is calcium disodium EDTA.

(43) The product of any one of (37) to (42), which comprises between about 0.5% and about 25% by weight of the water-soluble formulation.

(44) The product of (43), which comprises between about 1.0% and about 5% by weight of the water-soluble formulation.

(45) The product of any one of (37) to (44), which further comprises one or more of: terpenes, terpenoids, flavonoids, viscosity modifiers, natural emulsifiers, oils, thickening agents, minerals, acids, bases, vitamins, flavours, colourants, sweeteners, and combinations thereof.

(46) The product of any one of (37) to (45), wherein from about 25 mL to about 500 mL of the product provides an intoxicating effect equivalent to a standard British unit of alcohol.

(47) The product of (46), which provides for the intoxicating effect in from about 10 minutes to about 120 minutes after use.

(48) The product of (47), wherein the intoxicating effect lasts for a time period of from about 30 minutes to about 300 minutes after use.

(49) The product of any one of (37) to (48), which is shelf-stable for at least 55 days.

(50) The product of any one of (37) to (49), which loses less than 20% by weight of the cannabinoid or cannabis-derived compound in 3 months.

(51) The product of (50), which loses less than 16% by weight THC content in 70 days.

(52) The product of any one of (37) to (51), wherein the cannabinoid or cannabis-derived compound is stable.

(53) The product of any one of (37) to (52), wherein at least 80% by weight of the cannabinoid or cannabis-derived compound remains present after about 2 months at a temperature between about 17° C. and about 40° C.

(54) The product of (53), wherein at least 90% by weight of the cannabinoid or cannabis-derived compound remains present after about 2 months at a temperature between about 17° C. and about 40° C.

(55) The product of any one of (37) to (52), wherein at least 84.89% by weight of the cannabinoid or cannabis-derived compound remains present after about 3 months at a temperature of about 40° C.

(56) The product of any one of (37) to (55), which has an oxygen content of between about 0 ppm and about 500 ppm.

(57) A method for preparing the water-soluble formulation of any one of (1) to (34), the method comprising mixing, in any order, a cannabinoid or a cannabis-derived compound with a glycerin-based carrier surfactant and an emulsifier to prepare the water-soluble formulation.

(58) The method according to (57), comprising: mixing the cannabinoid or a cannabis-derived compound with a carrier oil until a homogenous mixture is formed; and mixing the glycerin-based carrier surfactant and the emulsifier into the homogenous mixture to prepare the water-soluble formulation.

(59) The method according to (58), comprising mixing the cannabinoid or cannabis-derived compound and carrier oil in heated conditions.

(60) The method according to (59), wherein the heated conditions are a temperature between about 40° C. and about 50° C.

(61) The method according to any one of (57) to (60), further comprising mixing a sucrose monoester into the homogenous mixture.

(62) The method according to any one of (57) to (61), further comprising microfluidizing the water-soluble formulation to obtain a particle size of between about 30 nm and about 100 nm.

(63) The method according to (62), wherein the microfluidizing provides a particle size of about 40 nm.

(64) A method for preparing a product comprising the water-soluble formulation of any one of (1) to (34), the method comprising: mixing, in any order, a cannabinoid or a cannabis-derived compound with a glycerin-based carrier surfactant and an emulsifier to prepare the water-soluble formulation; and mixing the water-soluble formulation with an aqueous solution.

(65) The method according to (64), comprising: mixing the cannabinoid or the cannabis-derived compound and a carrier oil until a homogenous mixture is formed; mixing the glycerin-based carrier surfactant and the emulsifier into the homogenous mixture to prepare the water-soluble formulation; and mixing the water-soluble formulation with the aqueous solution.

(66) The method according to (65), wherein the aqueous solution comprises one or more of: terpenes, terpenoids, flavonoids, viscosity modifiers, natural emulsifiers, oils, thickening agents, minerals, acids, bases, vitamins, flavours, colourants, sweeteners, and combinations thereof.

(67) The method according to any one of (65) to (66), further comprising mixing a sucrose monoester into the homogenous mixture.

(68) The method according to any one of (64) to (67), further comprising adding a chelating agent to the aqueous solution.

(69) The method according to (68), wherein the chelating agent is calcium disodium EDTA.

(70) A water-soluble formulation comprising a cannabinoid or a cannabis-derived compound, a carrier oil, a surfactant, and an emulsifier, wherein the water-soluble cannabis formulation is transparent, translucent, or pearlescent when mixed with an aqueous solution.

(71) The water-soluble formulation of (70), which is as further defined in any one of (1) to (34).

(71) A water-soluble formulation comprising a cannabinoid or a cannabis-derived compound; an emulsifier comprising Quillaja saponins; a carrier oil, and a glycerin-based carrier surfactant.

(72) The water-soluble formulation of (71), wherein the emulsifier comprising Quillaja saponins is a Q-NATURALE® emulsifier, Saponin from Quillaja Bark, Saponin Quillaja sp., a Quillaja extract, purified Quillaja saponins, or any combination thereof.

(73) The water-soluble formulation of (72), wherein the emulsifier comprising Quillaja saponins is a Q-NATURALE® emulsifier.

(74) The water-soluble formulation of (71), wherein the carrier oil is comprised of monoglycerides or medium chain triglycerides.

(75) The water-soluble formulation of (74), wherein the carrier oil is comprised of monoglycerides, and the monoglycerides comprise glyceryl monostearate, glyceryl hydroxystearate, glyceryl monoleate, winterized glyceryl monoleate, monolaurin, glyceryl monolinoleate, or any combination thereof.

(76) The water-soluble formulation of (71), which comprises up to 10% by weight of the cannabinoid or cannabis-derived compound, up to 15% by weight of the emulsifier comprising Quillaja saponins, and up to 10% by weight of the carrier oil.

(77) The water-soluble formulation of (71), which comprises a ratio of glycerin-based carrier surfactant:emulsifier comprising Quillaja saponins of between about 25:1 and about 5:1.

(78) The water-soluble formulation of (71), which comprises a ratio of glycerin-based carrier surfactant:carrier oil of between about 75:1 and about 25:1.

(79) The water-soluble formulation of (71), wherein the cannabinoid is cannabinoid is THC (Δ9-THC), Δ8-THC, trans-Δ10-THC, cis-Δ10-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT, cannabicitran, or any combination thereof.

(80) The water-soluble formulation of (71), wherein the cannabinoid is THC.

(81) The water-soluble formulation of (71), wherein the cannabinoid is CBD.

(82) The water-soluble formulation of (71), wherein the glycerin-based carrier surfactant is a vegetable glycerin.

(83) The water-soluble formulation of (71), which comprises between about 80% and about 90% by weight of the glycerin-based carrier surfactant.

(84) The water-soluble formulation of (71), further comprising one or more additives selected from the group consisting of terpenes, terpenoids, flavonoids, viscosity modifiers, natural emulsifiers, oils, thickening agents, minerals, acids, bases, vitamins, flavours, colourants, sweeteners, and combinations thereof.

(85) A beverage comprising the water-soluble formulation of (71).

(86) The beverage of (85), which comprises one or more salts.

(87) The beverage of (86), which is a sports drink beverage.

(88) The beverage of (87), which comprises between about 0.5% and about 25% by weight of the water-soluble formulation.

(89) The beverage of (88), which comprises between about 5 mg/mL and about 25 mg/mL of CBD.

(90) The beverage of (87), which is in Tetra Pak™0 packaging.

(91) A method for preparing the water-soluble formulation of claim 1, the method comprising: mixing a cannabinoid or a cannabis-derived compound with a carrier oil to provide a cannabinoid mixture; mixing a glycerin-based carrier surfactant and an emulsifier comprising Quillaja saponins to provide a glycerin-based mixture; and combining the cannabinoid mixture and the glycerin-based mixture under high shear homogenization to prepare the water-soluble formulation.

(92) The method according to (91), wherein the emulsifier comprising Quillaja saponins is a Q-NATURALE® emulsifier, Saponin from Quillaja Bark, Saponin Quillaja sp., a Quillaja extract, purified Quillaja saponins, or any combination thereof.

(93) The method according to (91), wherein the emulsifier comprising Quillaja saponins is a Q-NATURALE® emulsifier.

(94) The method according to (91), wherein the cannabinoid is CBD.

(95) The method according to (91), wherein the cannabinoid is THC.

### EXAMPLES

The following examples are included to demonstrate various embodiments of the present disclosure. It should be appreciated by those of skill in the art that the techniques disclosed in the examples that follow represent techniques discovered by the inventors to function well in the practice of the present disclosure, and thus may be considered to constitute preferred modes for its practice. However, those of skill in the art should, in light of the present disclosure, appreciate that many changes can be made in the specific embodiments which are disclosed and still obtain a like or similar result without departing from the scope of the present disclosure.

### Example 1

In this Example, water-soluble formulations including various cannabinoid distillates for use in a beverage were prepared.

Initially, 1.5 g of cannabinoid distillate was mixed with 2.0 g of Maisine CC and the mixture was then warmed using a microwave oven. Aliquots of 0.05 g of beta-pinene, 0.15 g of limonene, 0.0125 g of hexyl acetate, 0.15g of terpinolene, and 0.1375 g of beta-caryophyllene were then added to the cannabinoid/Maisine CC mixture and the mixture was stirred to incorporate these ingredients. Once the above components were thoroughly mixed, 194 g of glycerin was added, and 2.0 g ALCOLEC® F-100 was added slowly with stirring. This mixture was then homogenized and put into a microfluidizer to form an emulsion with very small particles (<100 nm). The resulting water-soluble formulation was optically clear.

The various cannabinoid distillates were: Bakerstreet (pure 100% Indica strain, commercially available from Canadian LP Tweed), which includes THC only; Houndstooth (a sativa-dominant strain, commercially available from Canadian LP Tweed), which includes THC only; and Penelope (a hybrid strain, commercially available from Lift & Co.), which includes both THC and CBD in ratios of 1.5:1, THC:CBD.

### Example 2

In this Example, water-soluble formulations for use in a beverage were prepared and analyzed for stability.

The following formulations were prepared using the methods of Example 1.

The formulations were then mixed with purified water to prepare a beverage product.

| | | |
|---|---|---|
| Component | Amount | Unit |

Water-Soluble Formulation A (100 g)

| | | |
|---|---|---|
| β-Pinene | 90 | mg |
| Nerolidol | 45 | mg |
| Isopulegol | 45 | mg |
| gamma-Terpinene | 45 | mg |
| Bakerstreet Terpenes | 25 | Mg |
| Cannabis Distilate | 750 | Mg |
| (Bakerstreet) | | |
| ALCOLEC ® F100 | 1000 | Mg |
| Maisine CC | 1000 | Mg |
| Glycerol | 97 | G |

Beverage Product with Formulation A (30 mL)

| | | |
|---|---|---|
| β-Pinene | 0.27 | mg |
| Nerolidol | 0.13 | mg |
| Isopulegol | 0.13 | mg |
| gamma- | 0.13 | mg |
| Terpinene | | |
| Bakerstreet | 0.07 | mg |
| Terpenes | | |
| Cannabis | 2.22 | mg |
| Distilate | | |
| (Bakerstreet) | | |
| ALCOLEC ® F100 | 2.96 | mg |
| Maisine CC | 2.96 | mg |
| Glycerol | 0.29 | g |
| Purified Water | 29.70 | g |

| | | |
|---|---|---|
| Component | Amount | Unit |

Water-Soluble Formulation B (100 g)

| | | |
|---|---|---|
| Eugenol | 45 | mg |
| p-Cymeme | 22.5 | mg |
| Humulene | 22.5 | mg |
| Terpinolene | 67.5 | mg |
| β-Caryophyllene | 67.5 | mg |
| Houndstooth | 25 | mg |
| Terpenes | | |
| Cannabis Distilate | 750 | mg |
| (Houndstooth) | | |
| ALCOLEC ® F100 | 1000 | mg |
| Maisine CC | 1000 | mg |
| Glycerol | 97 | g |

Beverage Product with Formulation B (30 mL)

| | | |
|---|---|---|
| Eugenol | 0.13 | mg |
| p-Cymeme | 0.07 | mg |
| Humulene | 0.07 | mg |
| Terpinolene | 0.20 | mg |
| β-Caryophyllene | 0.20 | mg |
| Houndstooth | 0.07 | mg |
| Terpenes | | |
| Cannabis Distilate | 2.22 | mg |
| (Houndstooth) | | |
| ALCOLEC ® F100 | 2.96 | mg |
| Maisine CC | 2.96 | mg |
| Glycerol | 0.29 | g |
| Purified Water | 29.70 | g |

| | | |
|---|---|---|
| Component | Amount | Unit |

Water-Soluble Formulation C(100 g)

| | | |
|---|---|---|
| Limonene | 56.25 | mg |
| Hexyl Acetate | 11.25 | mg |
| Terpinolene | 90 | mg |
| β-Caryophyllene | 67.5 | mg |
| Penelope Terpenes | 25 | mg |
| Cannabis Distilate | 750 | mg |
| (Penelope) | | |
| ALCOLEC ® F100 | 1000 | mg |
| Maisine CC | 1000 | mg |
| Glycerol | 97 | g |

Beverage Product with Formulation C (30 mL)

| | | |
|---|---|---|
| Limonene | 0.33 | mg |
| Hexyl Acetate | 0.07 | mg |
| Terpinolene | 0.53 | mg |
| β-Caryophyllene | 0.40 | mg |
| Penelope | 0.15 | mg |
| Terpenes | | |
| Cannabis | 4.44 | mg |
| Distilate | | |
| (Penelope) | | |
| ALCOLEC ® | 5.93 | mg |
| F100 | | |
| Maisine CC | 5.93 | mg |
| Glycerol | 0.57 | g |
| Purified Water | 29.41 | g |

When the water-soluble formulation was mixed into the purified water, it resulted in a fully transparent, optically clear mixture.

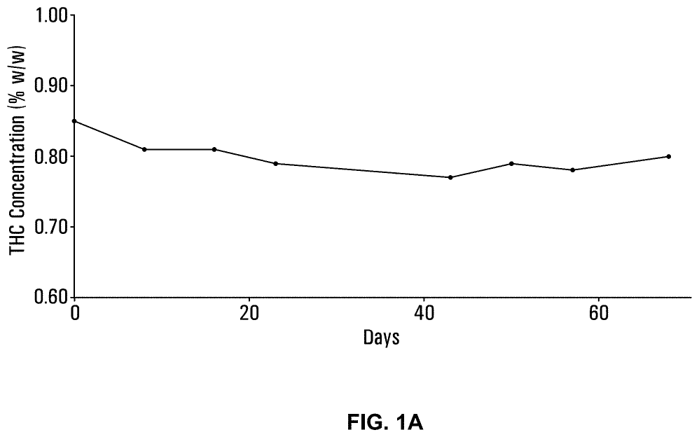

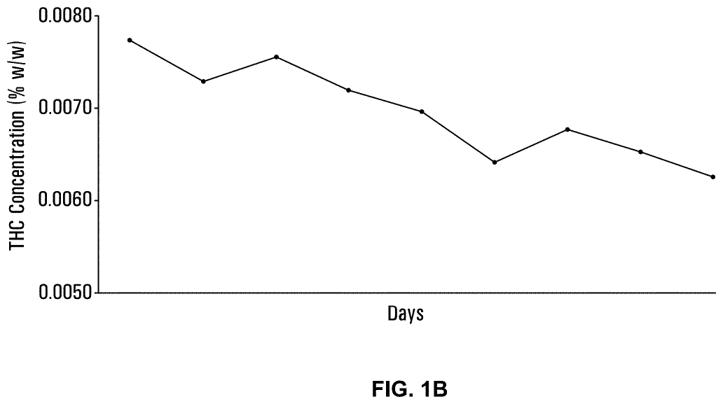

Stability of the water-soluble formulation and end use beverage for Formulation A were monitored for 71 days. The formulations and beverages were kept at 17° C. and sampled once per week. The results are listed in the Table below and the data in the table is illustrated in FIGS. 1A & 1B.

THC Concentration (% w/w)

| | Water-Soluble | Beverage Product |
|---|---|---|
| Days | Formulation | with Formulation |
| 0 | 0.8100 | 0.0077 |
| 8 | 0.8500 | 0.0073 |
| 16 | 0.8100 | 0.0076 |
| 23 | 0.8100 | 0.0072 |
| 43 | 0.7900 | 0.0070 |
| 50 | 0.7700 | 0.0064 |
| 57 | 0.7900 | 0.0068 |
| 68 | 0.7800 | 0.0065 |
| 71 | 0.8000 | 0.0063 |

As shown in the table and FIGS. 1A & 1B, the THC concentration remained relatively stable over a period of 71 days. That is the formulation lost less than 16% by weight THC content in 70 days.

### Example 3

In this Example, the experiential effect and organoleptic properties of formulations of THC distillate was determined.

500 grams of cannabis from Bakerstreet (Relax), Penelope (Enhance/Do), and Houndstooth (socialize) was cold ethanol extracted to produce a resin. The resin was processed by short path distillation to produce a distillate free from volatile organic compounds and other impurities. Each drink trial used 1.0 gram of distillate for formulation to be prepared. Formulation of the drink syrup was done two days prior to each session to allow time for testing and CoA generation. The drink syrup was diluted the day of the trial to 2 mg/30 mL in MilliQ water. One strain was tested per session and each strain was tested a minimum of two times. Trials took place on the same day and the same time for 6 weeks to 12 weeks. Participants were required to remain onsite for one hour following dose administration to ensure safety, as well as timely and accurate recording of experience.

For organoleptic testing, drink formulations were compared against a placebo to confirm that the tastes were indistinguishable. Participants did not consume the drinks to minimize THC ingestion. A binary preference based test similar to a paired-comparison test was used. Each participant sampled one placebo and one cannabis drink and rated which they thought contained cannabis. Results were compared across the entire study group and analyzed by binomial test. The 95% confidence interval was calculated.

For experiential testing, dosages of 6 mg/person THC (or 3×30 mL volumes of 2 mg per 30 mL volume) were taken at once. Tombstone data (gender, weight, tolerance, experience, etc.) was collected for each participant. Self-reporting of intoxication was obtained by journal entry and cognitive impairment was obtained using a mobile app (Otorize). Journal entry and cognitive impairment were collected in parallel to track experience at 15 minute intervals with a baseline established directly before testing. Cognitive impairment testing measuring reaction time, decision making, time estimation, motor tracking, and balance were obtained using Druid (www.druidapp.com). For self-reporting, categories were created to match marketing expectations and divided in the “positive” and “negative” words (see, Table). The sum of each rating for each domain was calculated for both positive and negative words for each participant in order to minimize reporting bias. Each week, participants completed a self-reporting survey at different set time points beginning at baseline and followed for 5 hours after consumption.

TABLE
intoxication trial designs

| | | |
|---|---|---|
| Mood | Happy | Sad |
| | Giggly | Gloomy |
| | Merry | Serious |
| | Joyful | Down |
| | Relaxed | Anxious |
| | Euphoric | Depressed |
| Energy | Exhilarated | Fatigued |
| | Alert | Groggy |
| | Energetic | Tired |
| | Lively | Lethargic |
| | Hyper | Lazy |
| | Peppy | Sleepy |
| Social | Talkative | Zoned Out |
| | Articulate | Incoherent |
| | Vocal | Quiet |
| | Disinhibited | Paranoid |
| | Open | Gaurded |
| | Expressive | Dull |
| Intoxication | High | Burnt Out |
| | Drunk | Disorriented |
| | Stoned | Sober |
| | Intoxicated | Sick |
| | Buzzed | Clearheaded |
| | Hungry | Nauseated |
| Work | Productive | Forgetful |
| | Focused | Scattered |
| | Creative | Stow |
| | Motivated | Bored |
| | Imaginative | Confused |
| | Inventive | Apathetic |
| Physical | Warm | Cold |
| | Tingly | Itchy |
| | Numb | Sore |
| | Comfortable | Restless |
| | Heavy | Jittery |
| | Cozy | Irritated |

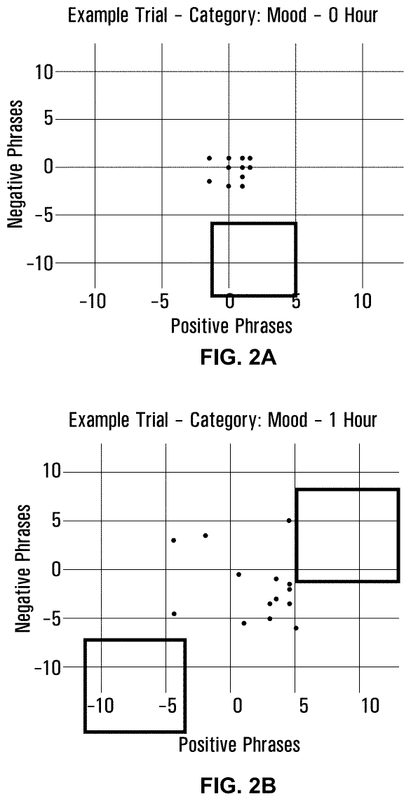

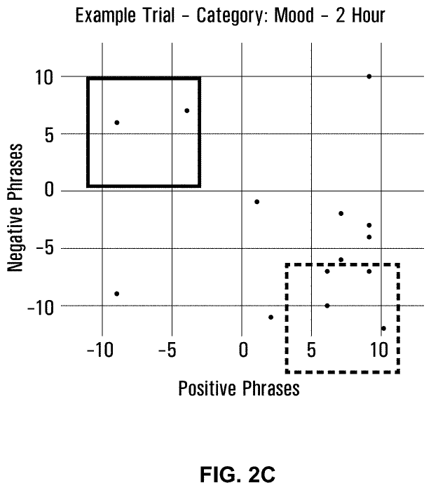

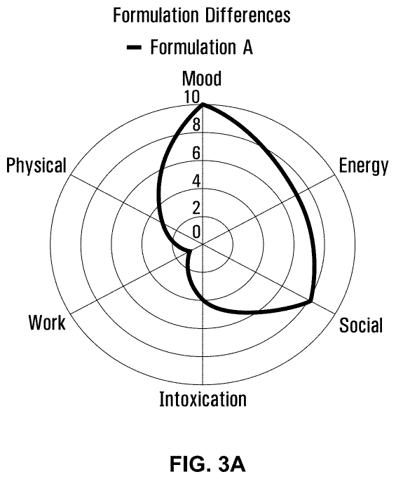

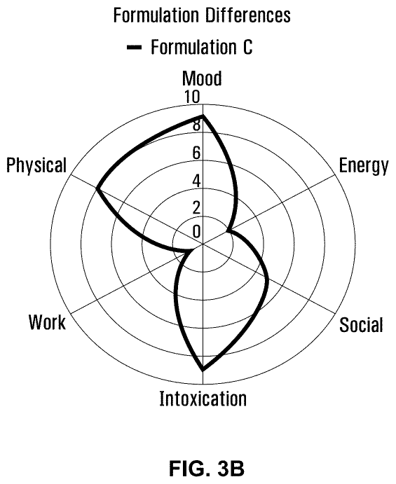

Participants rated how they felt in each category (mood, energy, social, intoxication level, work, and physical) on a scale of −3 to +3. Three stages of intoxication were analyzed including sober, elevated, and intoxicated. Intoxication levels over time were reported on a scale from 1(Sober) to 10 (the most intoxicated one could imagine). Ratings were then summed across the negative and positive words for a category to create an average minimizing bias. The average was then plotted in each category for each member of the test group following a modified diamond of opposites test (see, FIGS. 2A-2C). The resulting point (x, y) was plotted for each participant and domain to create 6 distinct scatter-plot matrices where the x- axis and y-axis represented the positive words and negative words respectfully associated with the given domain. Modified diamond of opposites test was done on the average scores for each category to track how the experience changed over time. Data that was contradictory was discarded from the analysis. Data points (excluding contradictory points for each domain) were also summed together to create a vector with some magnitude that was used to generate polar area charts to compare gestalt results across all categories. The magnitude and direction of this vector was calculated and used to create polar area or radar charts to show the complete experience in each category for each trial across the study and to compare the differences between the 3 stages of intoxication studied. These charts were compared over time to see how the experience evolved or between formulations to identify ensemble effects. Individual word pairs were also analyzed and used to generate polar area and radar charts (see, FIGS. 3A-3B). The direction of these vectors indicated a positive or negative association between the THC-infused drinks and the domains.

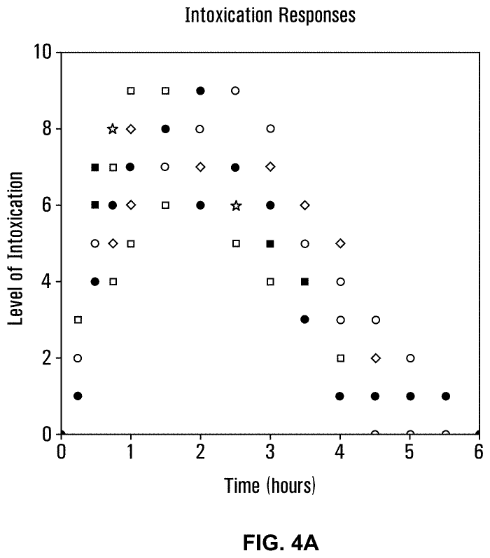

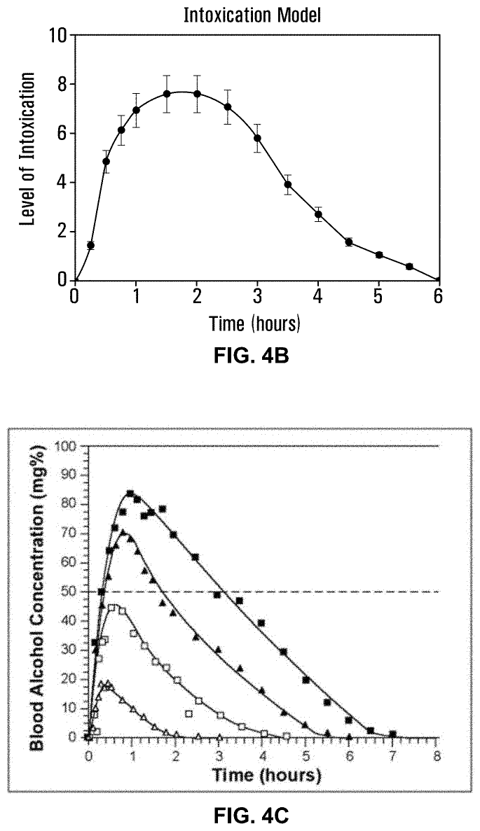

Intoxication as measured using the mobile app or by self-reporting was used to create a model for intoxication by the formulations and compared to preexisting models for alcohol intoxication (see, FIGS. 4A-4C). This data was used to develop additional formulations.

As shown in FIG. 2A, the box contains the normal baseline variability in experience without intoxication. Values outside this box were considered “of interest” for analysis and/or possible follow up. As shown in FIG. 2B, the boxes contained contradictions (self reports of conflicting subjective experience). Values outside these boxes were considered “of interest” for analysis and/or possible follow up. As shown in FIG. 2C, the box in the upper left contains negative results (bad outcomes in some category). The box in the lower right of FIG. 2C contains positive results (good outcomes in some category).

Formulation A provided primarily mood, energy, and social experiences (FIG. 3A). Formulation C provided primarily mood, physical, intoxication, and social experiences (FIG. 3B).

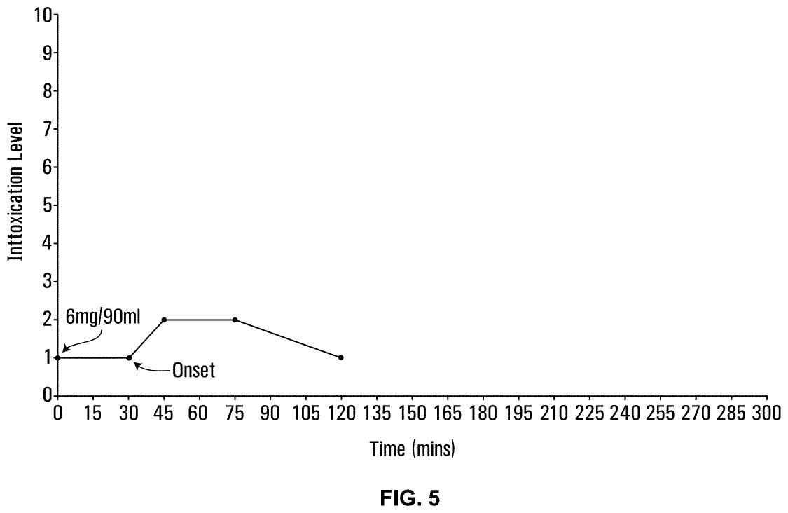

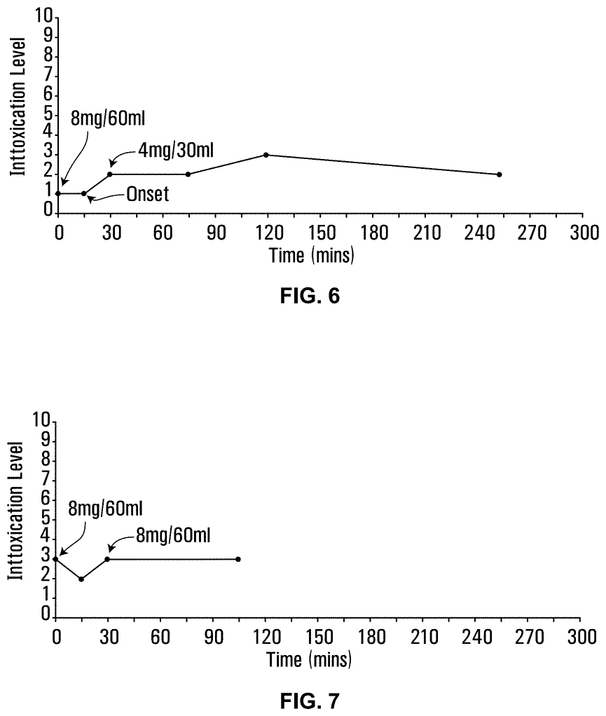

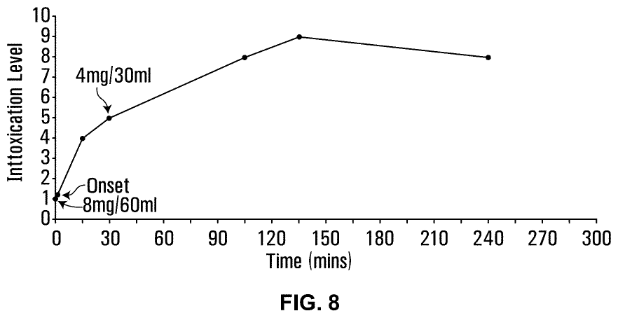

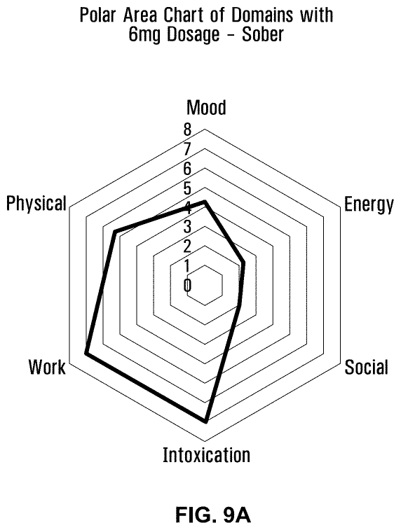

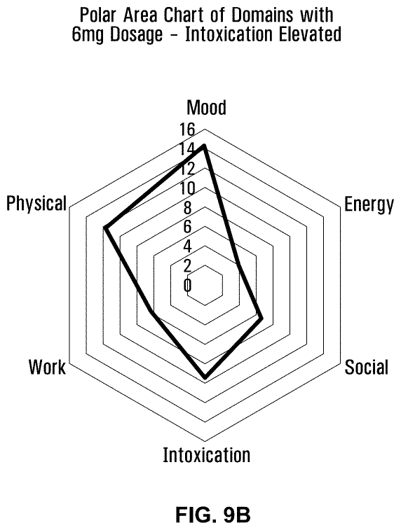

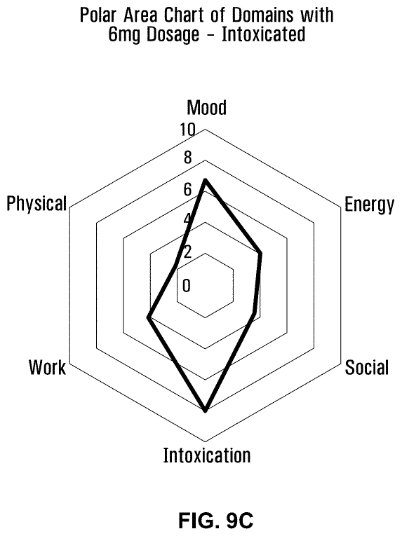

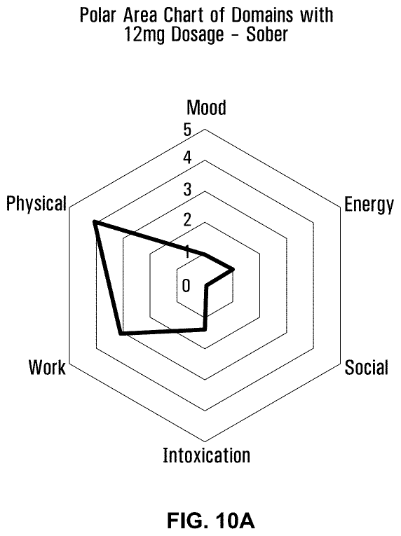

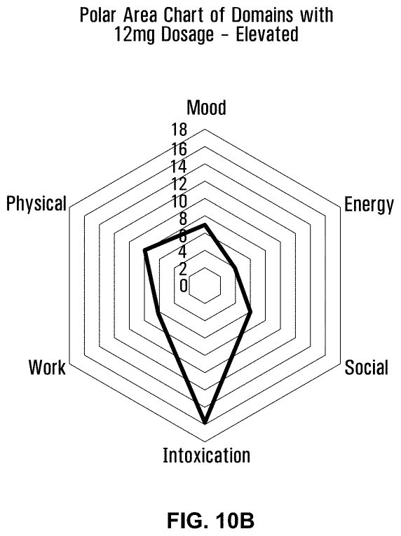

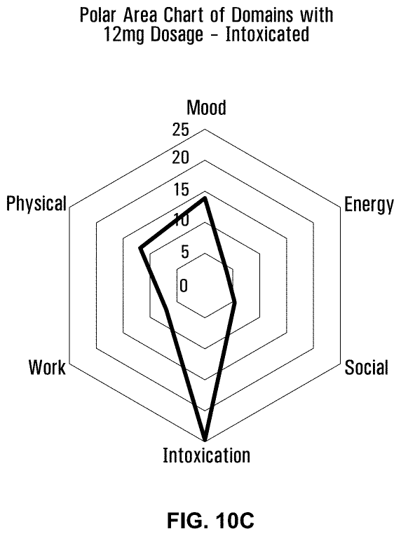

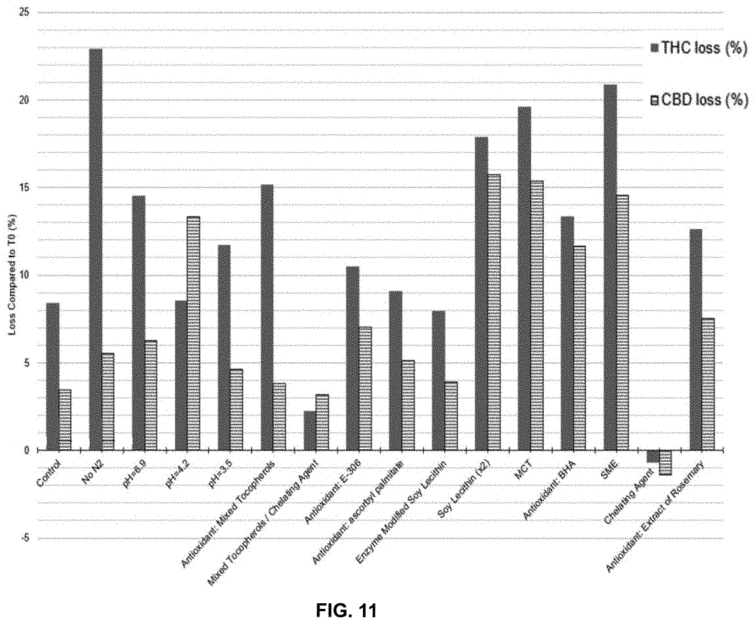

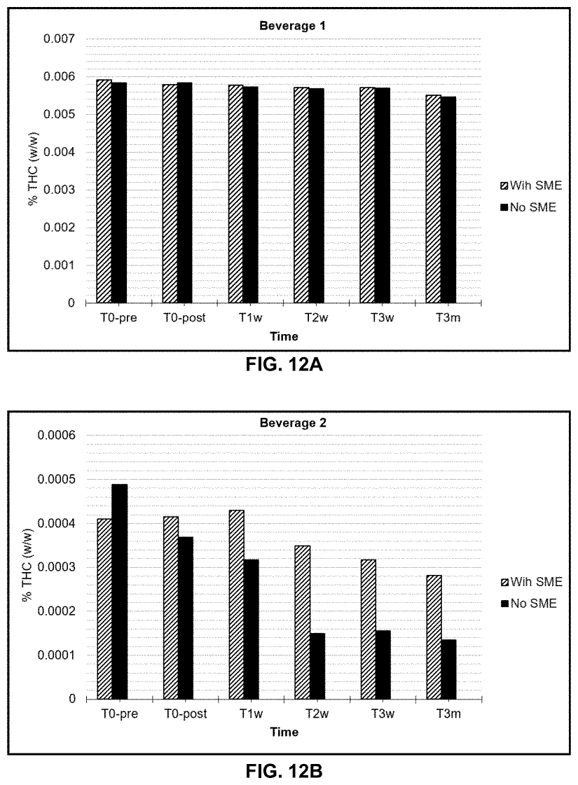

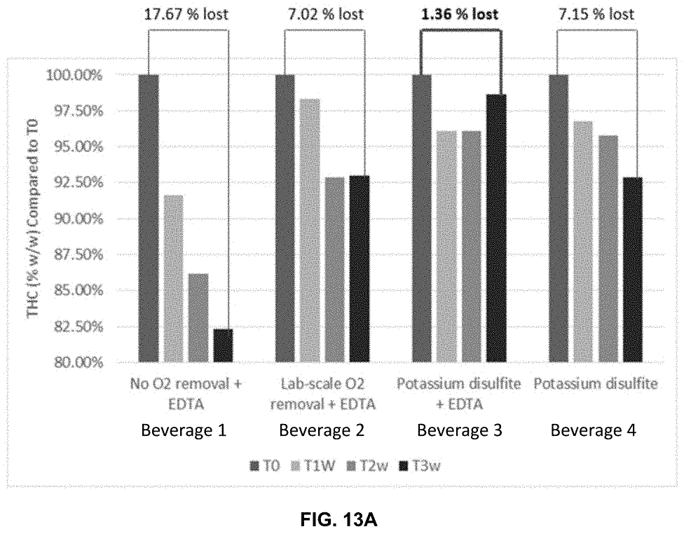

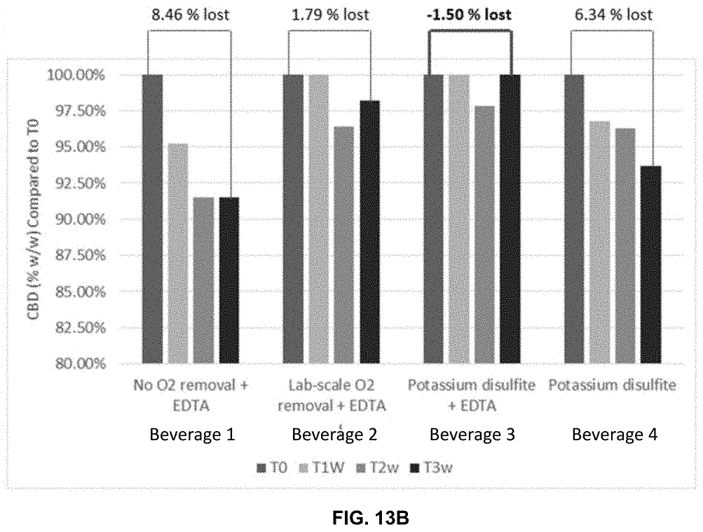

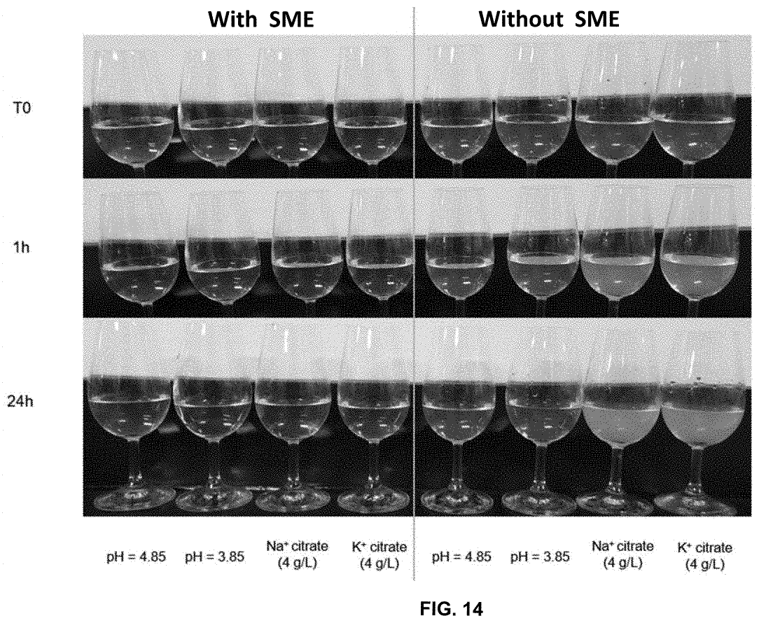

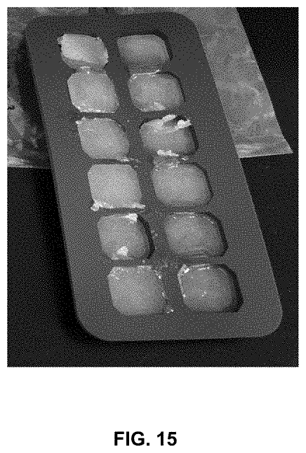

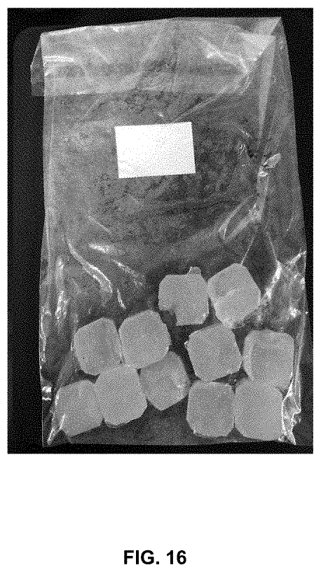

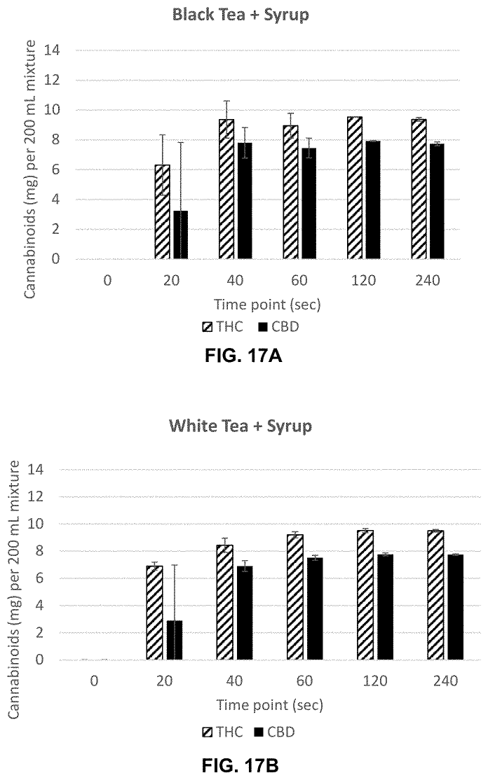

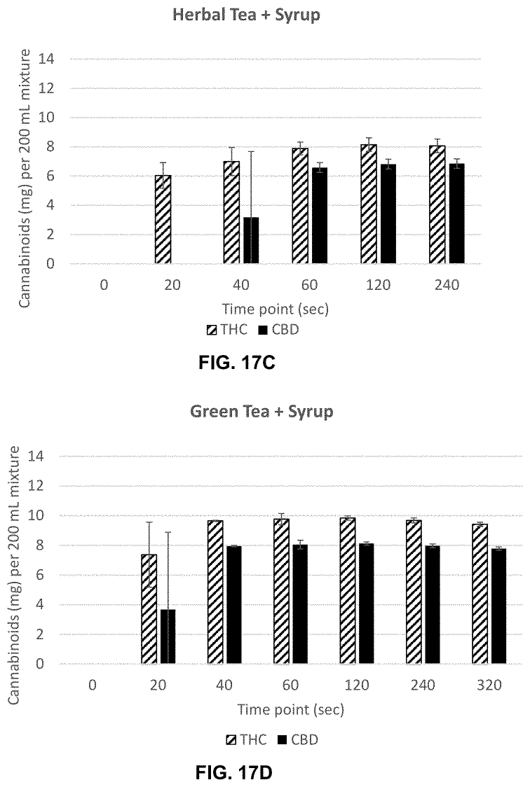

FIG. 5 shows the intoxication level over time for a participant with cannabis tolerance administered a 6 mg dose of a formulation using Bakerstreet strain without terpenes. FIG. 6 shows the intoxication level over time for a participant with cannabis tolerance administered a 12 mg dose of a formulation using Penelope strain with terpenes. FIG. 7 shows the intoxication level over time for a participant with cannabis tolerance administered a 16 mg dose of a formulation using Penelope strain with terpenes and esters. FIG. 8 shows the intoxication level over time for a participant without cannabis tolerance administered a 12 mg dose of a formulation using Penelope strain with terpenes and esters. FIGS. 9A-9C are polar area charts showing the three levels of intoxication for 6 mg doses of a formulation using Bakerstreet strain without terpenes. FIGS. 10A-10C are polar area charts showing the three levels of intoxication for 12 mg doses of a formulation using Penelope strain with terpenes and esters.

These results demonstrated that the resultant vectors calculated from modified diamond of opposites tests could be used to indicate positive or negative association between the THC- infused drinks and the domains studied. For the 6 mg dose of Bakerstreet without terpenes the data indicated a potential positive correlation between intoxication level and mood, energy, social, and intoxication domain. A potential negative correlation between intoxication level and work, and a varying correlation between intoxication level and physical depended on the level of intoxication. For the 12 mg dose of Penelope with terpenes and esters the data indicated a potential positive correlation between intoxication level and mood, energy, intoxication domain, and physical. A potential negative correlation between intoxication level and social and a varying correlation between intoxication level and work depended on the level of intoxication.

The following are additional exemplary water-soluble formulations of the present disclosure:

| | | | | | | | | | Concentration of | |
|---|---|---|---|---|---|---|---|---|---|---|
| | | Carrier | Carrier | | Emulsifier | | | | Antioxidant in | |
| Distillate | Terpenes | Oil | Oil | Emulsifier | Amount | Glycerin | Water | Antioxidant | Formulation | Ethanol |
| (g) | (g) | Type | Amount | Type | (g) | (g) | (g) | Used | (PPM) | (g) |
| 2.0 | 0.0 | Maisine CC | 2.0 | Alcolec F100 | 2.0 | 194.0 | 0.0 | N/A | 0.0 | 0.0 |
| 2.0 | 0.0 | Maisine CC | 4.0 | Alcolec F100 | 2.0 | 192.0 | 0.0 | N/A | 0.0 | 0.0 |
| 2.0 | 0.0 | Maisine CC | 2.0 | Alcolec F100 | 4.0 | 192.1 | 0.0 | N/A | 0.0 | 0.0 |
| 2.0 | 0.0 | Maisine CC | 4.0 | Alcolec F100 | 4.0 | 190.1 | 0.0 | N/A | 0.0 | 0.0 |
| 4.0 | 0.0 | Maisine CC | 2.0 | Alcolec F100 | 2.0 | 192.0 | 0.0 | N/A | 0.0 | 0.0 |
| 4.0 | 0.0 | Maisine CC | 4.0 | Alcolec F100 | 2.0 | 190.0 | 0.0 | N/A | 0.0 | 0.0 |
| 4.0 | 0.0 | Maisine CC | 2.0 | Alcolec F100 | 4.0 | 190.0 | 0.0 | N/A | 0.0 | 0.0 |
| 4.0 | 0.0 | Maisine CC | 4.0 | Alcolec F100 | 4.0 | 188.1 | 0.0 | N/A | 0.0 | 0.0 |
| 2.0 | 0.0 | Maisine CC | 2.0 | Alcolec F100 | 2.0 | 194.0 | 0.0 | N/A | 0.0 | 0.0 |
| 2.0 | 0.0 | Maisine CC | 2.0 | Alcolec F100 | 2.0 | 190.0 | 4.0 | N/A | 0.0 | 0.0 |
| 2.0 | 0.0 | Maisine CC | 2.0 | Alcolec F100 | 2.0 | 184.0 | 10.0 | N/A | 0.0 | 0.0 |
| 2.0 | 0.0 | Maisine CC | 2.0 | Alcolec F100 | 2.0 | 174.0 | 20.0 | N/A | 0.0 | 0.0 |
| 2.0 | 0.0 | Maisine CC | 2.0 | Alcolec F100 | 2.0 | 154.0 | 40.1 | N/A | 0.0 | 0.0 |
| 2.0 | 0.0 | Maisine CC | 2.0 | Alcolec F100 | 2.0 | 190.0 | 4.1 | Alpha | 200.0 | 0.0 |
| | | | | | | | | Tocopherol | | |
| 2.0 | 0.0 | Maisine CC | 2.0 | Alcolec F100 | 2.0 | 190.0 | 4.1 | Alpha | 100.0 | 0.0 |
| | | | | | | | | Tocopherol | | |
| 2.0 | 0.0 | Maisine CC | 2.0 | Alcolec F100 | 2.0 | 190.0 | 4.1 | Alpha | 50.0 | 0.0 |
| | | | | | | | | Tocopherol | | |
| 2.0 | 0.0 | Labrasol | 2.0 | Alcolec F100 | 2.0 | 194.0 | 0.0 | N/A | 0.0 | 0.0 |
| 2.0 | 0.0 | Labrafac | 2.0 | Alcolec F100 | 2.0 | 194.0 | 0.0 | N/A | 0.0 | 0.0 |
| | | Lipophile | | | | | | | | |
| | | WL 1349 | | | | | | | | |
| 2.0 | 0.0 | Labrafil | 2.0 | Alcolec F100 | 2.0 | 194.0 | 0.0 | N/A | 0.0 | 0.0 |
| | | M1944 | | | | | | | | |
| 2.0 | 0.0 | Peceol | 2.0 | Alcolec F100 | 2.0 | 194.0 | 0.0 | N/A | 0.0 | 0.0 |
| 2.0 | 0.0 | Plurol | 2.0 | Alcolec F100 | 2.0 | 194.0 | 0.0 | N/A | 0.0 | 0.0 |
| | | Oliqiue CC | | | | | | | | |
| | | 497 | | | | | | | | |
| 2.0 | 0.0 | No Carrier | 0.0 | Alcolec F100 | 2.0 | 196.0 | 0.0 | N/A | 0.0 | 0.0 |
| | | Oil used | | | | | | | | |
| 2.0 | 0.0 | Maisine CC | 2.0 | Alcolec F100 | 2.0 | 194.0 | 0.0 | N/A | 0.0 | 0.0 |
| 2.0 | 0.0 | Maisine CC | 2.0 | Alcolec F100 | 2.0 | 193.0 | 0.0 | N/A | 0.0 | 1.0 |
| 2.0 | 0.0 | Maisine CC | 2.0 | Alcolec F100 | 2.0 | 189.7 | 5.0 | N/A | 0.0 | 0.0 |
| 2.0 | 0.0 | Maisine CC | 2.0 | Alpha-Tocopherol | 2.0 | 194.0 | 0.0 | N/A | 0.0 | 0.0 |
| | | | | Conjugate | | | | | | |
| 2.0 | 0.0 | Maisine CC | 2.0 | Alpha-Tocopherol | 2.0 | 0.0 | 194.0 | N/A | 0.0 | 0.0 |
| | | | | Conjugate | | | | | | |
| 0.5 | 0.0 | Maisine CC | 2.0 | Alcolec F100 | 2.0 | 195.5 | 0.0 | N/A | 0.0 | 0.0 |
| 2.0 | 0.0 | (1:1) | 2.0 | Alcolec F100 | 2.0 | 194.0 | 0.0 | N/A | 0.0 | 0.0 |
| | | Solution of | | | | | | | | |
| | | Maisine CC | | | | | | | | |
| | | and Labrasol | | | | | | | | |
| 2.0 | 0.0 | (9:1) | 2.0 | Alcolec F100 | 2.0 | 194.0 | 0.0 | N/A | 0.0 | 0.0 |
| | | Solution of | | | | | | | | |
| | | Labrasol and | | | | | | | | |
| | | Maisine CC | | | | | | | | |
| 2.0 | 0.0 | Maisine CC | 2.0 | Alcolec F100 | 2.0 | 194.0 | 0.0 | N/A | 0.0 | 0.0 |
| 2.0 | 0.0 | Labrafil | 2.0 | Alcolec F100 | 2.0 | 194.0 | 0.0 | N/A | 0.0 | 0.0 |
| | | M1944 | | | | | | | | |
| 2.0 | 0.0 | (1:1.5) | 2.0 | Alcolec F100 | 2.0 | 194.0 | 0.0 | N/A | 0.0 | 0.0 |
| | | Solution of | | | | | | | | |
| | | Maisine CC | | | | | | | | |
| | | and Labrasol | | | | | | | | |
| 2.0 | 2.0 | N/A | 0.0 | Alcolec F100 | 2.0 | 194.0 | 0.0 | N/A | 0.0 | 0.0 |
| 1.5 | 0.5 | Maisine CC | 2.0 | Alcolec F100 | 2.0 | 154.0 | 0.0 | N/A | 0.0 | 0.0 |
| 0.0 | 0.0 | Maisine CC | 20.0 | (6:1) | 80.0 | 0.0 | 100.4 | N/A | 0.0 | 0.0 |
| | | | | Mixture of | | | | | | |
| | | | | Labrasol and | | | | | | |
| | | | | Plurol Olique CC | | | | | | |
| | | | | 497 | | | | | | |
| 0.0 | 0.0 | Maisine CC | 2.0 | (6:1) | 42.0 | 0.0 | 56.1 | N/A | 0.0 | 0.0 |
| | | | | Mixture of | | | | | | |
| | | | | Labrasol and Plurol | | | | | | |
| | | | | Olique CC 497 | | | | | | |
| 0.0 | 4.0 | Maisine CC | 4.0 | Alcolec F100 | 4.0 | 188.0 | 0.0 | N/A | 0.0 | 0.0 |
| 3.0 | 0.0 | Maisine CC | 3.1 | Alcolec F100 | 3.1 | 291.0 | 0.0 | N/A | 0.0 | 0.0 |
| 2.0 | 0.0 | Maisine CC | 2.0 | Alcolec F100 | 2.0 | 194.0 | 0.0 | Alpha | 413.0 | 0.0 |
| | | | | | | | | Tocopherol | | |
| 2.0 | 0.0 | Maisine CC | 2.0 | Alcolec F100 | 2.0 | 194.0 | 0.0 | Alpha | 206.0 | 0.0 |
| | | | | | | | | Tocopherol | | |
| 2.0 | 0.0 | Maisine CC | 2.0 | Alcolec F100 | 2.0 | 194.0 | 0.0 | Alpha | 106.0 | 0.0 |
| | | | | | | | | Tocopherol | | |
| 2.0 | 0.0 | Maisine CC | 2.0 | Alcolec F100 | 2.0 | 194.0 | 0.0 | N/A | 0.0 | 0.0 |
| 2.0 | 0.0 | Labrafac | 2.0 | Alcolec F100 | 2.0 | 194.1 | 0.0 | N/A | 0.0 | 0.0 |
| | | Lipophile | | | | | | | | |
| | | WL 1349 | | | | | | | | |
| 4.00 | 0.00 | Maisine CC | 4.00 | Alcolec F100 | 3.90 | 191.28 | 0.00 | N/A | 0.00 | 0.00 |
| 4.01 | 0.00 | Maisine CC | 4.01 | Alcolec F100 | 3.80 | 188.27 | 0.00 | N/A | 0.00 | 0.00 |
| 4.05 | 0.00 | Maisine CC | 4.03 | Alcolec F100 | 3.70 | 188.35 | 0.00 | N/A | 0.00 | 0.00 |
| 2.00 | 0.00 | Maisine CC | 2.00 | Alcolec F100 | 2.00 | 194.05 | 0.00 | N/A | 0.00 | 0.00 |
| 4.04 | 0.00 | Maisine CC | 4.04 | Alcolec F100 | 3.60 | 188.41 | 0.00 | N/A | 0.00 | 0.00 |
| 4.04 | 0.00 | Maisine CC | 4.02 | Alcolec F100 | 3.53 | 188.53 | 0.00 | N/A | 0.00 | 0.00 |
| 4.03 | 0.00 | Maisine CC | 4.08 | Alcolec F100 | 3.41 | 188.60 | 0.00 | N/A | 0.00 | 0.00 |
| 4.01 | 0.00 | Maisine CC | 4.06 | Alcolec F100 | 3.29 | 188.76 | 0.00 | N/A | 0.00 | 0.00 |
| 4.08 | 0.00 | Maisine CC | 4.01 | Alcolec F100 | 3.19 | 188.83 | 0.00 | N/A | 0.00 | 0.00 |
| 4.05 | 0.00 | Maisine CC | 4.02 | Alcolec F100 | 3.18 | 188.89 | 0.00 | N/A | 0.00 | 0.00 |
| 4.00 | 0.00 | Maisine CC | 4.01 | Alcolec F100 | 2.98 | 188.96 | 0.00 | N/A | 0.00 | 0.00 |
| 4.00 | 0.00 | Maisine CC | 4.01 | N/A | 0.00 | 192.00 | 0.00 | N/A | 0.00 | 0.00 |
| 3.98 | 0.00 | Maisine CC | 4.02 | Alcolec F100 | 2.84 | 189.18 | 0.00 | N/A | 0.00 | 0.00 |
| 4.06 | 0.00 | Maisine CC | 4.03 | Alcolec F100 | 2.70 | 189.53 | 0.00 | N/A | 0.00 | 0.00 |
| 2.00 | 0.00 | Maisine CC | 2.03 | Alcolec F100 | 1.99 | 193.97 | 0.00 | Quercetin | 110.40 | 0.00 |
| 2.05 | 0.00 | Maisine CC | 2.02 | Alcolec F100 | 2.00 | 193.99 | 0.00 | Quercetin | 208.90 | 0.00 |
| 2.03 | 0.00 | Maisine CC | 1.98 | Alcolec F100 | 2.02 | 194.01 | 0.00 | Quercetin | 405.90 | 0.00 |
| 4.07 | 0.00 | Maisine CC | 4.06 | Alcolec F100 | 2.56 | 189.45 | 0.00 | N/A | 0.00 | 0.00 |
| 4.01 | 0.00 | Maisine CC | 4.06 | Alcolec F100 | 2.39 | 189.58 | 0.00 | N/A | 0.00 | 0.00 |
| 4.03 | 0.00 | Maisine CC | 4.01 | Alcolec F100 | 2.24 | 189.76 | 0.00 | N/A | 0.00 | 0.00 |
| 4.01 | 0.00 | Maisine CC | 4.02 | Alcolec F100 | 2.11 | 189.90 | 0.00 | N/A | 0.00 | 0.00 |
| 4.00 | 0.00 | Maisine CC | 4.00 | Alcolec F100 | 1.95 | 190.04 | 0.00 | N/A | 0.00 | 0.00 |
| 4.01 | 0.00 | Maisine CC | 4.00 | Alcolec F100 | 1.77 | 190.24 | 0.00 | N/A | 0.00 | 0.00 |
| 4.01 | 0.00 | Maisine CC | 4.00 | Alcolec F100 | 1.50 | 190.52 | 0.00 | N/A | 0.00 | 0.00 |
| 4.01 | 0.00 | Maisine CC | 4.01 | Alcolec F100 | 1.25 | 190.74 | 0.00 | N/A | 0.00 | 0.00 |
| 4.01 | 0.00 | Maisine CC | 4.02 | Alcolec F100 | 1.00 | 191.00 | 0.00 | N/A | 0.00 | 0.00 |
| 4.01 | 0.00 | Maisine CC | 3.98 | Alcolec F100 | 5.01 | 187.00 | 0.00 | N/A | 0.00 | 0.00 |
| 4.02 | 0.00 | Maisine CC | 4.00 | Alcolec F100 | 6.01 | 186.00 | 0.00 | N/A | 0.00 | 0.00 |
| 3.00 | 1.00 | Maisine CC | 4.00 | Alcolec F100 | 3.99 | 187.97 | 0.00 | N/A | 0.00 | 0.00 |
| 4.03 | 0.00 | Tween 80 | 4.02 | Alcolec F100 | 4.00 | 188.00 | 0.00 | N/A | 0.00 | 0.00 |
| 4.02 | 0.00 | Maisine CC | 4.05 | Rhamnolipids | 1.00 | 187.01 | 4.01 | N/A | 0.00 | 0.00 |
| | | | | (>90%) | | | | | | |
| 3.03 | 1.01 | Maisine CC | 4.05 | Alcolec F100 | 4.01 | 188.01 | 0.00 | N/A | 0.00 | 0.00 |
| 2.04 | 0.00 | Maisine CC | 2.00 | Alcolec F100 | 2.03 | 194.10 | 0.00 | Alpha | 400 / 100 | 0.00 |
| | | | | | | | | Tocopherol / | | |
| | | | | | | | | Quercetin | | |
| 2.00 | 0.00 | Maisine CC | 2.01 | Alcolec F100 | 2.03 | 194.01 | 0.00 | Tocoblend | 200.00 | 0.00 |
| | | | | | | | | GT-10 | | |
| 2.04 | 0.00 | Maisine CC | 2.06 | Alcolec F100 | 2.01 | 194.03 | 0.00 | Tocoblend | 200.00 | 0.00 |
| | | | | | | | | AR | | |
| 2.00 | 0.00 | Maisine CC | 2.00 | Alcolec F100 | 2.00 | 194.00 | 0.00 | Tocoblend | 200.00 | 0.00 |
| | | | | | | | | ATR | | |
| 2.00 | 0.00 | Maisine CC | 1.99 | Alcolec F100 | 1.99 | 194.03 | 0.00 | Parolox | 200.00 | 0.00 |
| 2.00 | 0.00 | Maisine CC | 2 | Alcolec FH20 | 2 | 194 | 0 | N/A | N/A | N/A |

### Example 4

In the present example, aqueous solutions were prepared using water-soluble formulations of the present disclosure. The aqueous solutions were analyzed for stability under accelerated temperature conditions (40° C). The purpose of this example was to determine the effectiveness of surfactants, emulsifiers, and stabilizers (e.g. antioxidants, chelating agents, etc.) in preventing loss of cannabinoids (e.g. due to oxidation) in aqueous solutions containing the water-soluble formulations. The turbidity of the solutions was also determined.

Water-soluble formulations were prepared in accordance with Table 1 below. The control formulation contained approximately equivalent by weight amounts of cannabinoid distillate, monoglycerides (carrier oil) and soy lecithin (emulsifier), in a vegetable glycerin (carrier surfactant). The pH studies were conducted by adding suitable pH adjusters (e.g. potassium citrate to adjust pH between 4.0 and 4.5). Other modifications to the control formulation were made as noted as “Difference from Control”. The formulations were then mixed with purified water to prepare the aqueous solution with a target THC concentration of about 0.007% w/w and a target CBD concentration of about 0.0059% w/w. The resulting aqueous solutions were all sparged with nitrogen gas unless otherwise indicated (i.e. “No N₂”).

The cannabinoid content of each aqueous solution was determined after two weeks and the loss of cannabinoid content after two weeks relative to the initial cannabinoid content was calculated. The results are listed in Table 1 below and shown in FIG. 11.

TABLE 1

| | | | | THC | CBD |
|---|---|---|---|---|---|
| Water-Soluble | | Turbidity | | loss | loss |
| Formulation | Difference from Control | (NTU) | pH | (%) | (%) |
| Control | None | 15.35 | 4.23 | 8.39 | 3.47 |
| No N₂ | Not sparged with N₂ | 16.48 | 4.28 | 22.88 | 5.53 |
| pH = 6.9 | pH | 5.67 | 6.92 | 14.50 | 6.27 |
| pH = 4.2 | pH | 5.32 | 4.20 | 8.54 | 13.33 |
| pH = 3.5 | pH | 5.84 | 3.49 | 11.69 | 4.65 |
| Antioxidant: Mixed | Blend of Tocopherols added to w-s | 17.88 | 4.27 | 15.12 | 3.81 |
| Tocopherols | emulsion at 20:1 w/w ratio to soy lecithin | | | | |
| | emulsifier | | | | |
| Mixed Tocopherols | Blend of Tocopherols added to w-s | 21.8 | 4.46 | 2.23 | 3.17 |
| (Antioxidant)/ | emulsion at 20:1 w/w ratio to soy lecithin | | | | |
| Chelating Agent | emulsifier. | | | | |
| (EDTA) | | | | | |
| | EDTA added to aqueous solution in | | | | |
| | minor amount | | | | |
| Antioxidant: E-306 | Antioxidant added to w-s emulsion at 1:1 | 17.29 | 4.14 | 10.46 | 7.04 |
| | w/w ratio to soy lecithin emulsifier | | | | |
| Antioxidant: | Antioxidant added to w-s emulsion at | 15.55 | 4.25 | 9.07 | 5.12 |
| ascorbyl palmitate | 0.06:1 w/w ratio to soy lecithin emulsifier | | | | |
| Enzyme Modified | Replaced soy lecithin emulsifier with | 3.43 | 4.38 | 7.95 | 3.91 |
| Soy Lecithin | enzyme modified lyso lecithin | | | | |
| Soy Lecithin | Increased quantity of soy lecithin | 24.5 | 4.38 | 17.85 | 15.72 |
| | emulsifier to 2:1 w/w to monoglyceride | | | | |
| | carrier oil | | | | |
| MCT | Replaced monoglyceride carrier oil with | 21.0 | 4.15 | 19.59 | 15.38 |
| | MCT oil | | | | |
| Antioxidant: BHA | Antioxidant added to w-s emulsion at | 8.99 | 4.20 | 13.35 | 11.66 |
| | 0.02:1 w/w ratio to soy lecithin emulsifier | | | | |
| Sucrose monoester | Sucrose monopalmitate added to w-s | 5.34 | 4.3 | 20.84 | 14.56 |
| (SME) | emulsion at 1:1 w/w ratio to soy lecithin | | | | |
| | emulsifier | | | | |
| Chelating Agent | EDTA added to aqueous solution in | 16.11 | 4.38 | −0.68 | −1.39 |
| (EDTA) | minor amount | | | | |
| Antioxidant: | Antioxidant added to w-s emulsion at | 16.02 | 4.41 | 12.59 | 7.52 |
| Extract of | 1.6:1 w/w ratio to soy lecithin emulsifier | | | | |
| Rosemary | | | | | |

As seen in Table 1 above, the presence of oxygen (no N₂ purge) increased the loss of THC and CBD relative to the control sample. Surprisingly, the addition of antioxidants either had a negligible effect or increased the loss of THC and CBD relative to the control sample. Also surprisingly, a chelating agent (EDTA), in the absence of antioxidant, significantly decreased the loss of both THC and CBD. The combination of a chelating agent and an antioxidant (mixed tocopherols) somewhat decreased the amount of THC lost after two weeks, however the effect is small compared to the effect of a chelating agent alone. Also, as compared to the effect of the mixed tocopherol antioxidant alone, which increased the loss of THC, the addition of a chelating agent was able to counteract the negative effect of the antioxidant and actually prevented the loss of THC.

From this, it can be concluded that oxygen contributes to the degradation of cannabinoids in aqueous solutions containing the water-soluble formulation. Furthermore, it appears that antioxidants are less effective than a chelating agent, which had a drastic effect in preventing the degradation of cannabinoids in aqueous solutions containing the water-soluble formulation of the present disclosure.

Development of stable emulsions is complex. It is noteworthy in this example that the combination of soy lecithin emulsifier and sucrose monoester provided significantly improved clarity to the aqueous solution, having a turbidity of only 5.34. This suggests that sucrose monoesters may be useful in strengthening the emulsion.

Thus, the example shows that a chelating agent and sucrose monoester aid in strengthening the emulsion and preventing cannabinoid loss (e.g. due to oxidation). Other components tested were not as effective.

### Example 5

In the present example, actual beverage formulations (e.g. flavour and carbonation) were used to test the effectiveness of water-soluble formulations of the present disclosure. The purpose of this example was to determine the effect of sucrose monoester (SME) in strengthening the emulsion properties of water-soluble formulations of the present disclosure, when used in beverages in which a chelating agent (EDTA) was added.

The water-soluble formulations contained approximately equivalent by weight amounts of cannabinoid distillate, monoglyceride (carrier oil) and soy lecithin (emulsifier), in vegetable glycerin (carrier surfactant). Sucrose monoester (SME; sucrose monopalmitate) at about a 1:1 ratio with the soy lecithin was included in the indicated formulations as shown in Table 2 below (i.e. Beverages 1 a and 2a). The water-soluble formulations, both with and without SME, were optically clear (translucent).

Beverages were prepared by mixing in the water-soluble formulations. Beverage 1 was formulated with a target THC concentration of 0.005% w/w using either the water-soluble formulation with SME (“la”) or without SME (“1b”). Beverage 2 was formulated with a target THC concentration of 0.0004% w/w using either the water-soluble formulation with SME (“2a”) or without SME (“2b”). Chelating agent was added at a minor amount to the aqueous beverage solution, and the beverages were carbonated. When the water-soluble formulations were mixed into the beverage liquid, it resulted in a fully transparent mixture having the colour of the beverage liquid.

The actual cannabinoid content of each beverage was determined prior to and after pasteurization, and then weekly thereafter for 1 week (Tlw), 2 weeks (T2w) and 3 weeks (T3w), and at 3 months (T3M). The results are listed in Table 2 below and shown in FIGS. 12A and 12B.

TABLE 2

| | | % THC (w/w) | | | | | |
|---|---|---|---|---|---|---|---|
| | SME | T0- | T0- | | | | |
| Beverage | Included | prepast | postpast | T1w | T2w | T3w | T3m |
| 1a | Yes | 0.00592 | 0.00579 | 0.00578 | 0.00570 | 0.00570 | 0.00550 |
| 1b | No | 0.00584 | 0.00584 | 0.00573 | 0.00567 | 0.00569 | 0.00546 |
| 2a | Yes | 0.000410 | 0.000415 | 0.000429 | 0.000349 | 0.000318 | 0.000282 |
| 2b | No | 0.000488 | 0.000369 | 0.000317 | 0.000150 | 0.000156 | 0.0001345 |

As seen in Table 2 above, in the beverage containing the higher THC concentration, the use of a chelating agent (EDTA) with the water-soluble formulation was sufficient at protecting against THC degradation as the results for beverages la and 1b are similar. This suggests that the chelating agent was effective in preventing degradation of the THC. However, in the beverage with the lower THC concentration, the inclusion of SME in the water-soluble formulation greatly enhanced the protection against THC degradation. This suggests that the combination of soy lecithin and SME has a role in strengthening the emulsion and preventing undesired release of cannabinoids from the emulsion.

Turbidity of the beverages was also measured and is shown in Table 3 below.

TABLE 3

| | | % THC (w/w) | | | | |
|---|---|---|---|---|---|---|
| | | Turbidity | Turbidity | Turbidity | Turbidity | Turbidity |
| | | at | at | at | at | at |
| | SME | T0-prepast | T0-postpast | T1w | T2w | T3w |
| Beverage | (Included) | (NTU) | (NTU) | (NTU) | (NTU) | (NTU) |
| 1a | Yes | 13.17 | 11.92 | 10.88 | 10.06 | 10.48 |
| 1b | No | 17.46 | 16.66 | 15.08 | 16.90 | 15.48 |
| 2a | Yes | 0.51 | 0.59 | 0.39 | 0.40 | 0.41 |
| 2b | No | 2.28 | 2.25 | 3.62 | 2.19 | 3.52 |

As can be seen from Table 3 above, all of the beverages had decent clarity, with the highest measured turbidity being below 20. Notably however, the beverages made using the water-soluble formulation containing the combination of soy lecithin and SME improved the clarity of the beverages over the water-soluble formulations containing soy lecithin alone.

Thus, this example shows that a water-soluble formulation comprising both SME and soy lecithin, in a beverage with a chelating agent added thereto, promotes stability of the cannabinoids in the beverage.

Notably, the beverages prepared herein using the water-soluble formulations of the present disclosure also had a ‘clean’ taste in that they did not taste like a cannabis plant, but rather had the desired added flavouring. Also, the addition of SME surprisingly did not cause the beverages to have an excessively undesirable bitter taste.

### Example 6

In the present example, aqueous solutions containing water-soluble formulations of the present disclosure were prepared and analyzed for stability over varying oxygen concentrations. The purpose of this example was to determine the effectiveness of water-soluble formulations comprising soy lecithin and sucrose monoester (SME), with beverages containing a chelating agent, in maintaining the stability of cannabinoids (e.g. THC and CBD) over varying oxygen concentrations in aqueous environments.

The water-soluble formulations contained approximately equivalent by weight amounts of cannabinoid distillate, monoglyceride (carrier oil), soy lecithin (emulsifier) and SME (emulsifier), in vegetable glycerin (carrier surfactant). Beverages were prepared by mixing the water-soluble formulations into an aqueous beverage medium.

Each of the beverages contained between 5.6 and 6.2 ppm THC and between 4.3 and 5.2 ppm CBD. Beverage 1 was subjected to lab-scale removal of oxygen. Beverages 2-4 were sparged with nitrogen, with beverages 3 and 4 further containing 175 ppm potassium disulfite to consume excess oxygen. All of the beverages were adjusted to a pH of approximately 4.4 using citric acid/potassium citrate, and a chelating agent was added as in previous examples to beverages 1-3 at a minor amount.

The cannabinoid content of each beverage was determined prior to and after pasteurization, and then weekly thereafter. Samples were held at accelerated temperatures (40° C.) and stored in amber glass bottles (355 mL capacity). The results are listed in Table 4 below and shown in FIG. 13A (THC) and FIG. 13B (CBD).

TABLE 4

| | | |
|---|---|---|
| Max | | |
| Dissolved | % THC Remaining | % CBD Remaining |

| Beverage | O₂ (ppb) | T0 | T1w | T2w | T3w | T0 | T1w | T2w | T3w |
|---|---|---|---|---|---|---|---|---|---|
| 1 | 8014 | 100.0 | 91.6 | 86.1 | 82.3 | 100.0 | 95.2 | 91.5 | 91.5 |
| 2 | 2666 | 100.0 | 98.3 | 92.8 | 93.0 | 100.0 | 101.8 | 96.4 | 98.2 |
| 3 | 442 | 100.0 | 96.1 | 96.1 | 98.6 | 100.0 | 101.4 | 97.8 | 101.5 |
| 4 | 688 | 100.0 | 96.8 | 95.8 | 92.8 | 100.0 | 96.8 | 96.3 | 93.7 |

Although it was thought that the presence of water might be detrimental to cannabinoid stability, the water-soluble formulations of the present disclosure maintained the cannabinoids in the aqueous environment, indicating excellent stability in an aqueous environment. Rather, stability was more closely related to oxygen concentration.

As can be seen in Table 4 above, oxygen contributes directly to the rate of cannabinoid degradation, particularly for THC. Beverage 2, which was not sparged with nitrogen and held the highest concentration of oxygen, showed the greatest loss of cannabinoids over time. Beverages 3 and 4, which contained potassium metabisulfite to scavenge residual oxygen, had the lowest concentration of oxygen and the slowest rate of cannabinoid degradation, with beverage 3 that additionally contained the chelating agent showing the best results for both THC and CBD.

Thus, the example shows that limiting oxygen concentrations in beverages, in combination with using a water-soluble formulation of the present disclosure comprising soy lecithin and SME, provides excellent stability of cannabinoids. Even with significant quantities of oxygen present (Beverage 2), the water-soluble formulation still performed exceptionally well, retaining over 80% of the THC and over 90% of the CBD after nearly a month.

### Example 7

In the present example, the clarity of beverages prepared using the water-soluble formulations of the present disclosure was observed under different pH and ionic conditions.

Aqueous solutions were prepared having different pH and ionic conditions: (1) pH 4.85, (2) pH 3.85, (3) Na+citrate (4 g/L), and (4) K⁺ citrate (4 g/L). To these aqueous solutions was added an equivalent amount of either:
(A) a water-soluble formulation containing approximately equivalent by weight amounts of cannabinoid distillate, monoglyceride (carrier oil), and soy lecithin (emulsifier), in vegetable glycerin (carrier surfactant); or
(B) a water-soluble formulation containing approximately equivalent by weight amounts of cannabinoid distillate, monoglyceride (carrier oil), soy lecithin (emulsifier) and SME (emulsifier), in vegetable glycerin (carrier surfactant).

The aqueous solutions were observed over the course of 24 hours. Images of the aqueous solutions at time 0 (“T0”), 1 hour (“T1”) and 24 hours (“T24”) can be seen in FIG. 14. Both water-soluble formulations exhibited decent performance at 1 hour at both pH 4.85 and pH 3.85. However, the soy lecithin and SME combination provided better clarity performance at high ionic conditions and over longer periods of time. Across all beverages, water-soluble formulations comprising both soy lecithin and SME exhibited a turbidity of less than 5.0 NTU.

### Example 8

In the present example, a gummy product was prepared using a water-soluble formulation of the present disclosure. The purpose of the example was to confirm that the water-soluble formulations could be successfully incorporated into a gummy product.

The gummy base was prepared with the following ingredients:

| Ingredient | wt % |
|---|---|
| Pectin | 1.91 |
| Cream of tartar | 1.72 |
| Citric Acid (Anhydrous) | 1.05 |
| Sugar | 57.20 |
| Glucose syrup | 9.53 |
| Water | 28.60 |
| Total | 100.0 |

The gummy base was found to provide both a good gummy set and texture. The gummies held up well after sitting at ambient temperatures (e.g. 17-18° C.) for 1 week, and could be readily separated from each other. It was found to be a suitable gummy base.

To prepare gummies containing cannabinoids, the water-soluble formulation was added to the gummy base and a gummy product was prepared therefrom. The water-soluble formulation contained approximately equivalent by weight amounts of THC cannabinoid distillate, monoglyceride (carrier oil), soy lecithin (emulsifier) and SME (emulsifier), in vegetable glycerin (carrier surfactant). The water-soluble formulation was added to the gummy base at a quantity of about 2.5 mg THC/3.5 g gummy.

The gummies set well after preparation. An image of the resulting gummy product is shown in FIG. 15. After 1 week at 25° C., the gummies had retained their shape (see FIG. 16). It is notable that the gummies prepared with the water-soluble formulations produced a qualitatively similar set to gummy base without the water-soluble formulation, even with the water-soluble formulation containing a high level of glycerin carrier surfactant. From this it may be concluded that the water-soluble formulation may be successfully incorporated into a gummy product.

### Example 9

In this example, water-soluble formulations prepared according to the present disclosure were added to standard tea bags comprising black tea, white tea, herbal tea and green tea leaves. The water-soluble formulations contained approximately equivalent by weight amounts of a cannabis concentrate, monoglyceride (carrier oil), and soy lecithin (emulsifier), in vegetable glycerin (carrier surfactant). The cannabis concentrate was a cannabis distillate having a 1.5:1 ratio of THC:CBD). The water-soluble formulation was added to the tea leaves in a tea bag at about a 10 mg quantity of THC or a 7.5 mg quantity of CBD. One tea bag for each type of tea was placed in boiling water and the beverage was left to steep (brew).

The brewed tea was observed visually and the overall THC concentration was assessed over time. Samples were taken at 0, 20, 40, 60, 120 and 240 seconds (and 320 seconds for green tea) after addition of boiling water. 2 duplicates were performed. The THC and CBD concentration in the different types of tea are shown in FIGS. 17A-17D for black tea (FIG. 17A), white tea (FIG. 17B), herbal tea (FIG. 17C) and green tea (FIG. 17D).

Overall, the tea bags dosed with a water-soluble formulation of the present disclosure showed good dispersibility of THC and CBD in the steeped tea beverage. After 40 seconds, over 80% of THC had dispersed in the tea beverage and remained (or increased further) throughout the trial. Herbal tea was a slight outlier, achieving 80% after 60 seconds. This may be due to plant interactions with the herbal tea. CBD also showed good dispersibility, but with slightly less of the CBD being dispersed in the tea (about 65-85%).

### Example 10

Certain beverages, such as those having a pH <5.0, can sometimes be more difficult to maintain stability of active ingredients, such as cannabinoids. In the present example, beverages containing water-soluble formulations of the present disclosure were prepared and analyzed for stability under accelerated conditions (i.e. at 40° C.).

Beverage #1 was a THC beverage having a target THC quantity of 2 mg, a lime/ginger flavour profile, and a pH of about 4.2.

Beverage #2 was a THC beverage having a target THC quantity of 10 mg, a bold and dark flavour/colour profile, and a pH of about 4.3.

Beverage #3 was a CBD beverage having a target CBD quantity of 20 mg, a cucumber/mint flavour profile, and a pH of about 4.26.

Beverage #4 was a THC beverage having a target THC quantity of 2 mg, a cucumber/mint flavour profile, and a pH of about 4.28.

The beverages were prepared using the water-soluble formulations of the present disclosure. Water-soluble formulations prepared according to the present disclosure were added to the beverages after the flavour and pH adjustments, by mixing the water-soluble formulation into each respective beverage. For beverages #1-3, the water-soluble formulations contained approximately equivalent by weight amounts of a cannabis concentrate, monoglyceride (carrier oil), soy lecithin (emulsifier), and SME (emulsifier), in vegetable glycerin (carrier surfactant). The cannabis concentrate was either a THC distillate or a CBD isolate. For beverage #4, the water-soluble formulation contained approximately equivalent by weight amounts of a THC distillate, monoglyceride (carrier oil), and soy lecithin (emulsifier), in vegetable glycerin (carrier surfactant). Beverage #4 did not contain SME.

One experiment was performed under laboratory conditions in which each beverage was subjected to lab-scale removal of oxygen, and packaged in cans for the duration of the experiment. It was determined experimentally that the dissolved oxygen content of these beverages was typically around about 229-1438 ppm. To simulate a commercial product, a second experiment was performed in which the beverages were bottled under conditions that removed oxygen, and sealed to prevent any exchange of gases between the inside and outside of the bottles.

The results of Experiment 1 are shown in Table 5 below, where the amount of THC and CBD in the beverage are represented as a percent difference from the target amount (% off spec). A positive value indicates the percentage of cannabinoid loss or percentage less than target. A negative value indicates the percentage above target. A problem occurred with the beverage 2, T2M preparation and this time point sample was discarded.

TABLE 5

| | Beverage 1 | Beverage 2 | Beverage 3 | Beverage 4 |
|---|---|---|---|---|
| Target: | 2 mg THC | 10 THC | 20 CBD | 2 THC |
| T0 (40° C.; % off spec) | −0.02 | −5.72 | 6.98 | −8.28 |
| T2w (40° C.; % off spec) | 7.52 | 16.23 | 8.19 | −3.17 |
| T1M (40° C.; % off spec) | 15.95 | 32.47 | 10.56 | −0.33 |
| T2M (40° C.; % off spec) | 13.38 | — | 14.58 | 19.55 |
| T3M (40° C.; % off spec) | 20.88 | 47.98 | 37.48 | 19.81 |

Stability testing at 40° C. represents accelerated stability testing. Generally, the 2-month data is roughly representative of 8-month stability at room temperature. 3-month data is roughly representative of 1-year stability at room temperature.

As can be seen in Table 5 above, beverages 1, 3 and 4 show very good stability at 2 months accelerated testing, with THC and CBD only being about 13%, 14% and 19% off spec, respectively. Factoring in the percentage off spec at TO, beverages 1, 3 and 4 show a loss of THC or CBD at 2-month accelerated testing of about 13.40%, 7.6% and 27.83%, respectively (e.g. T2M value-T0 value). Loss of THC and CBD appears to rise at the 3-month test point, particularly for beverages 1 and 3, perhaps due to the presence of oxygen in this lab-scale experiment. Beverage 2 shows a lesser degree of cannabinoid stability under these experimental conditions, which Experiment 2 suggests is due to the presence of oxygen in the lab-scale preparations.

The results of Experiment 2 are shown in Table 6 below, where again the amount of THC and CBD in the beverage are represented as a percent difference from the target amount (% off spec) as described above.

TABLE 6

| | Beverage 1 | Beverage 2 | Beverage 3 | Beverage 4 |
|---|---|---|---|---|
| Spec: | 2 mg THC | 10 mg THC | 20 mg CBD | 2 mg THC |
| T0 (40° C.; % off spec) | −2.51 | −10.11 | 4.79 | −9.70 |
| T2w (40° C.; % off spec) | −4.67 | −4.67 | 5.74 | −0.55 |
| T1M (40° C.; % off spec) | 3.17 | −2.06 | 8.42 | 8.23 |
| T2M (40° C.; % off spec) | 2.73 | — | 12.55 | 21.46 |
| T3M (40° C.; % off spec) | 1.13 | 5.00 | 13.50 | 26.51 |

As can be seen in Table 6 above, under bottled conditions representative of commercial packaging, the beverages showed excellent stability of cannabinoids. Most notably, the calculated THC and CBD loss at 3-months accelerated testing for beverages 1, 2 and 3 was only 3.64%, 15.11% and 8.71%, respectively (i.e. T3M value-T0 value). As representative of 1-year stability at room temperature, the water-soluble formulations of the present disclosure were found to provide significant cannabinoid stability in beverages under commercial conditions.

The outlier was beverage #4, which was about 21% and 26% off spec at T2M and T3M, respectively. Taking into account the T0 off spec value, beverage 4 showed THC loss of about 31.16% and 36.21% at T2M and T3M, respectively. Notably, beverage #4 did not contain the SME. Experimentally it was observed that this beverage became very turbid overtime, and this may have been due to instabilities within the flavour syrup that did not contain SME, which was found to help strengthen emulsions for certain flavour syrups. It is believed that this instability may have impacted the overall THC content by destabilizing the emulsification system leading to less protection of THC.

### Example 11

Certain beverages, such as those comprising salts, flavours, high dissolved oxygen levels, and/or which are packaged in containers lined with certain materials, such as forms of polyethylene (e.g. Tetra Pak®), may result in difficulties in maintaining stability of certain emulsifiers and/or the cannabinoids therein. Additionally, some beverage manufacturing facilities may not have readily available certain types of homogenization equipment, such as high pressure microfluidization equipment. Further, some beverages may require high temperature pasteurization which may present challenges for cannabinoid stability.

In the present example, water-soluble formulations comprising CBD isolate were formulated and prepared without the use of high pressure microfluidization and were analyzed for physical and chemical stability over time.

Different emulsifiers and stabilizers (e.g. an emulsifier comprising Quillaja saponins, sucrose monoester, Tween, Gum Acacia, PEG 40-Hydrogenated Castor oil) were investigated alone or in combination to prepare candidate water-soluble formulations using high shear homogenization. Infused sports drink beverages were then prepared using the water-soluble formulations and physical and chemical stability were evaluated (e.g. phase separation, aggregation/flaking, sedimentation, foaming, turbidity, and potency). From these studies, water-soluble formulations comprising an emulsifier comprising Quillaja saponins were identified as particularly suitable for use in a sports drink beverage and were further investigated.

An exemplary preparation of a water-soluble formulation comprising an emulsifier comprising Quillaja saponins is as follows: A glycerin-based carrier surfactant (GBCS) was mixed with the emulsifier comprising Quillaja saponins (EQS). In a separate vessel, CBD isolate was heated until melted. A monoglyceride carrier oil was added to the melted CBD isolate and mixed until a visually homogeneous mixture was obtained. The GBCS/EQS mixture was transferred to the container with the CBD isolate/carrier oil mixture and the combined mixture was homogenized until a visually homogeneous mixture was obtained.

In exemplary water-soluble formulations, the GBCS and EMS were in a weight ratio of between about 5:1 and about 10:1 (GBCS:EQS) and the GBCS and carrier oil were in a weight ratio of between about 35:1 and about 40:1 (GBCS:carrier oil). The CBD isolate was present at about 1.0% by weight.

The stability of two such exemplary water-soluble formulations containing an emulsifier comprising Quillaja saponins was evaluated by measuring CBD potency (% w/w) via high performance liquid chromatograph (HPLC) at different time points (0, 2, 3 and 13 weeks after preparation). Both water-soluble formulations were found to have acceptable chemical stability as determined by no or minimal change in CBD content after 13 weeks (Table 7).

TABLE 7

| | |
|---|---|
| Water-Soluble | CBD (% w/w) |

| Formulation | Initial | 2 weeks | 3 weeks | 13 weeks |
|---|---|---|---|---|
| A | 1.00 | 0.98 | 0.97 | 0.96 |
| B | 0.93 | 0.92 | 0.91 | 0.90 |

Physical stability was evaluated over 11 weeks and no apparent phase separation was observed in either formulation.

### Example 12

In this example, water-soluble formulations A and B from Example 11 were further investigated for physical and chemical stability in infused sports drink beverages. Specifically, water-soluble formulations were used to prepare CBD-infused sports drink beverages of three different flavours.

An exemplary infused sports drink beverage preparation protocol is as follows: Sports drink beverage liquid was dosed with water-soluble formulation A or B from Example 11 to a target potency of 20-22 mg CBD/500 mL. The infused sports drink beverage was heated in a water bath under conditions to replicate pasteurization and then placed in glass vials or Tetra Pak® containers for testing and evaluation.

To evaluate the stability in glass containers, the infused sports drink beverages (Flavour 1, Flavour 2, and Flavour 3) were prepared as described above using both water-soluble formulations A and B and stored in glass containers. The CBD content in the beverages was evaluated before pasteurization (T0 Pre), after pasteurization (T0 Post), and then over the course of 3 weeks with CBD potency (mg/500 mL) measured at week 1, week 2 and week 3. Acceptable formulation stability was obtained for all of the beverages, using both water-soluble formulations containing an emulsifier comprising Quillaja saponins (Table 8). The reason for the potency increase after P0 Post may be due to slight evaporation of beverage liquid.

TABLE 8

| | Beverage CDB (mg/500 mL) | | | | |
|---|---|---|---|---|---|
| | T0 Pre | T0 Post | 1 Week | 2 Weeks | 3 Weeks |
| | Water-Soluble Formulation A | | | | |
| Flavour 1 | 22.48 | 20.44 | 22.11 | 22.11 | 21.94 |
| Flavour 2 | 22.68 | 21.03 | 22.72 | 22.76 | 22.90 |
| Flavour 3 | 22.74 | 20.41 | 22.48 | 22.48 | 22.47 |
| | Water-Soluble Formulation B | | | | |
| Flavour 1 | 20.82 | 19.44 | 19.44 | 19.81 | 19.96 |
| Flavour 2 | 20.74 | 19.54 | 19.62 | 19.84 | 20.34 |
| Flavour 3 | 20.64 | 19.27 | 19.30 | 19.63 | 20.13 |

Visual observations for evidence of instability (e.g. sedimentation) were performed at regular intervals. In general, the beverages exhibited minimal sedimentation.

Based on the chemical and visual evaluations, stability trials in Tetra Pak® packaging were performed on sports drink beverages infused with exemplary water-soluble formulation B to determine compatibility with this type of commercial packaging. The inner contact layer of the Tetra Pak® is composed of LDPE, which presents a potential cannabinoid-scalping surface that could lead to decreased CBD potency and/or stability in the liquid. The effect of scalping was evaluated via potency analysis (measured by HPLC) on the beverages over the course of 6 weeks.

Sports drink beverages of Flavour 1, Flavour 2, and Flavour 3 were infused with water-soluble formulation B as described above and analyzed for CBD content over time in Tetra Pak® packaging with measurements taken after pasteurization (Post-Past), after addition to Tetra Pak® packaging and then at 1 week, 2 weeks and 6 weeks after addition to Tetra Pak® packaging (Table 9).

TABLE 9

| | Beverage CDB (mg/500 mL package) | | | | |
|---|---|---|---|---|---|
| | Water-Soluble Formulation B | | | | |
| | Post-Past.* | 0 Week | 1 Week | 2 Weeks | 6 Weeks |
| Flavour 1 | 21.53 | 19.42 | 18.84 | 18.49 | 18.13 |
| Flavour 2 | 22.32 | 20.72 | 19.25 | 19.06 | 18.49 |
| Flavour 3 | 21.70 | 19.57 | 19.57 | 18.63 | 18.29 |

*“Post-past”: represents potency after pasteurization but before addition to the Tetra Pak ®.

As can be seen, CBD loss occurred soon after the addition to Tetra Pak® packaging, but generally leveled off afterwards with only minimal further loss over time. The relatively slow decline and low levels of loss demonstrate that the water-soluble formulation B was acceptably stable in the infused beverage.

The candidate water-soluble formulations were successfully prepared with simple high shear homogenization without the need for sophisticated techniques (e.g. microfluidization or spray drying) and produced beverages comprising electrolytes that were acceptably stable when infused, without special procedures to exclude oxygen during infusion. The infused beverages using these candidate formulations of Example 11 were also found to be stable after they were pasteurized and when stored in commercial packaging (e.g. Tetra Pak®).

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A water-soluble formulation comprising a cannabinoid or a cannabis-derived compound; an emulsifier comprising Quillaja saponins; a carrier oil, and a glycerin-based carrier surfactant.

2. The water-soluble formulation of claim 1, wherein the emulsifier comprising Quillaja saponins is a Q-NATURALE® emulsifier, Saponin from Quillaja Bark, Saponin Quillaja sp., a Quillaja extract, purified Quillaja saponins, or any combination thereof.

3. The water-soluble formulation of claim 2, wherein the emulsifier comprising Quillaja saponins is a Q-NATURALE® emulsifier.

4. The water-soluble formulation of claim 1, wherein the carrier oil is comprised of monoglycerides or medium chain triglycerides.

5. The water-soluble formulation of claim 4, wherein the carrier oil is comprised of monoglycerides, and the monoglycerides comprise glyceryl monostearate, glyceryl hydroxystearate, glyceryl monoleate, winterized glyceryl monoleate, monolaurin, glyceryl monolinoleate, or any combination thereof.

6. The water-soluble formulation of claim 1, which comprises up to 10% by weight of the cannabinoid or cannabis-derived compound, up to 15% by weight of the emulsifier comprising Quillaja saponins, and up to 10% by weight of the carrier oil.

7. The water-soluble formulation of claim 1, which comprises a ratio of glycerin-based carrier surfactant:emulsifier comprising Quillaja saponins of between about 25:1 and about 5:1.

8. The water-soluble formulation of claim 1, which comprises a ratio of glycerin-based carrier surfactant:carrier oil of between about 75:1 and about 25:1.

9. The water-soluble formulation of claim 1, wherein the cannabinoid is cannabinoid is THC (Δ9-THC), Δ8-THC, trans-Δ10-THC, cis-Δ10-THC, THCA, THCV, Δ8-THCA, Δ9-THCA, Δ8-THCV, Δ9-THCV, THCVA, CBD, CBDA, CBDV, CBDVA, CBC, CBCA, CBCV, CBCVA, CBG, CBGA, CBGV, CBGVA, CBN, CBNA, CBNV, CBNVA, CBND, CBNDA, CBNDV, CBNDVA, CBE, CBEA, CBEV, CBEVA, CBL, CBLA, CBLV, CBLVA, CBT, cannabicitran, or any combination thereof.

10. The water-soluble formulation of claim 1, wherein the cannabinoid is THC.

11. The water-soluble formulation of claim 1, wherein the cannabinoid is CBD.

12. The water-soluble formulation of claim 1, wherein the glycerin-based carrier surfactant is a vegetable glycerin.

13. The water-soluble formulation of claim 1, which comprises between about 60% and about 97% by weight of the glycerin-based carrier surfactant.

14. The water-soluble formulation of claim 1, further comprising one or more additives selected from the group consisting of terpenes, terpenoids, flavonoids, viscosity modifiers, natural emulsifiers, oils, thickening agents, minerals, acids, bases, vitamins, flavours, colourants, sweeteners, and combinations thereof.

15. A beverage comprising the water-soluble formulation of claim 1.

16. The beverage of claim 15, which comprises one or more salts.

17. The beverage of claim 16, which is a sports drink beverage.

18. The beverage of claim 17, which comprises between about 0.1% and about 25% by weight of the water-soluble formulation.

19. The beverage of claim 18, which comprises between about 0.01 mg/mL and about 2.5 mg/mL of CBD.

20. The beverage of claim 17, which is in Tetra Pak™0 packaging.

21. A method for preparing the water-soluble formulation of claim 1, the method comprising:
mixing a cannabinoid or a cannabis-derived compound with a carrier oil to provide a cannabinoid mixture;
mixing a glycerin-based carrier surfactant and an emulsifier comprising Quillaja saponins to provide a glycerin-based mixture; and
combining the cannabinoid mixture and the glycerin-based mixture under high shear homogenization to prepare the water-soluble formulation.

22. The method according to claim 21, wherein the emulsifier comprising Quillaja saponins is a Q-NATURALS® emulsifier, Saponin from Quillaja Bark, Saponin Quillaja sp., a Quillaja extract, purified Quillaja saponins, or any combination thereof.

23. The method according to claim 21, wherein the emulsifier comprising Quillaja saponins is a Q-NATURALS® emulsifier.

24. The method according to claim 21, wherein the cannabinoid is CBD.

25. The method according to claim 21, wherein the cannabinoid is THC.
